SEQUENCE LISTING (132 sequences)

SEQ ID NO: 1 - protein, 1229 aa, Artificial Sequence
  synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKGGGGSGGGGSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSTGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 2 - DNA, 3690 bp, Artificial Sequence
  synthesized
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggcggagggaccaaggtggagatcaaaggcggtggcggtagtgggggaggcggttctggcggcggagggtccggcggtggaggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcgacaggcggtggagggtccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 3 - protein, 473 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 4 - DNA, 1422 bp, Artificial Sequence
  synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggagggtccggcggtggtggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagactctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagctga

SEQ ID NO: 5 - protein, 1229 aa, Artificial Sequence
  synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGCGTKVEIKGGGGSGGGGSGGGGSGGGGSEVQLVESGGGLVQPGGSLRLSCAASGFTISTNAMSWVRQAPGKCLEWIGVITGRDITYYASWAKGRFTISRDNSKNTLYLQMNSLRAEDTAVYYCARDGGSSAITSNNIWGQGTLVTVSTGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIKGGGGSGGGGSGRSLVESGGGLVQPGGSLRLSCTASGFTISSYHMQWVRQAPGKCLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGCGTKVEIK

SEQ ID NO: 6 - DNA, 3690 bp, Artificial Sequence
  synthesized
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggctgtgggaccaaggtggagatcaaaggtggaggcggttcaggcggaggtggaagtggtggtggcggctctggaggcggcggatctgaggtgcagctggtggagtctgggggaggcttggtccagcctggggggtccctgagactctcctgtgcagcctctggattcaccatcagtaccaatgcaatgagctgggtccgccaggctccagggaagtgcctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcgacaggcggtggagggtccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggttcaggcggaggtggaagtggtggtggcggctctggaggcggcggatctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaaggtggtggcggctctggaggaggagggtccggacggtcgctggtggagtctgggggaggcttggtccagcctggggggtccctgagactctcctgtactgcctctggattcaccatcagtagctaccacatgcagtgggtccggcaggcacctgggaagtgcctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacttggaacctggcgatgctgcaacttactattgtcagtctacctatcttggtactgattatgttggcggtgctttcggctgtgggaccaaggtggagatcaaatga

SEQ ID NO: 7 - protein, 473 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGCGTKVTVLGGGGSGGGGSGGGGSGGGGSQVQLQESGGGLVKPGGSLRLSCAASGFTFSSYWMSWVRQAPGKCLEWVANINRDGSASYYVDSVKGRFTISRDDAKNSLYLQMNSLRAEDTAVYYCARDRGVGYFDLWGRGTLVTVSS

SEQ ID NO: 8 - DNA, 1422 bp, Artificial Sequence
  synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggagggtccggcggtggtggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggctgcgggaccaaggtgaccgtcctaggtggaggcggttcaggcggaggtggttccggcggtggcggctccggtggaggcggctctcaggtgcaattgcaggagtcggggggaggcctggtcaagcctggagggtccctgagactctcctgtgcagcctctggattcacctttagtagttattggatgagctgggtccgccaggctccagggaagtgcctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctcgagctga

SEQ ID NO: 9 - protein, 1225 aa, Artificial Sequence
  synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLTVLGGGGSGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 10 - DNA, 3678 bp, Artificial Sequence
  synthesized
cagatcgtgctgagccagagccccgccatcctgagcgccagccccggcgagaaggtgaccatgacctgccgggccagcagcagcgtgagctacatccactggttccagcagaagcccggcagcagccccaagccctggatctacgccaccagcaacctggccagcggcgtgcccgtgcggttcagcggcagcggcagcggcaccagctacagcctgaccatcagccgggtggaggccgaggacgccgccacctactactgccagcagtggaccagcaacccccccaccttcggcggcggcaccaagctgaccgtgctgggtggtggtggctctggaggaggcgggagcgggggtggtggctcaggtggtggaggttcccaggtgcagctgcagcagcccggcgccgagctggtgaagcccggcgccagcgtgaagatgagctgcaaggccagcggctacaccttcaccagctacaacatgcactgggtgaagcagacccccggccggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgcccggagcacctactacggcggcgactggtacttcaacgtgtggggcgccggcaccaccgtgaccgtctcgagtggcggtggagggtccggcggtggtggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 11 - protein, 475 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 1428 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgggtggaggcggttcaggcggaggtggttccggcggtggcggctccggtggaggcggctctcaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagcggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaattcactctcaccatcagcagcctgcagcctgatgattttgcaacttattactgccaaggctatttttattttattagtcgtacttatgtaaattctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 13 - protein, 1218 aa, Artificial Sequence
  synthesized
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLTVLGGGGSGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 14 - DNA, 3657 bp, Artificial Sequence
  synthesized
cagatcgtgctgagccagagccccgccatcctgagcgccagccccggcgagaaggtgaccatgacctgccgggccagcagcagcgtgagctacatccactggttccagcagaagcccggcagcagccccaagccctggatctacgccaccagcaacctggccagcggcgtgcccgtgcggttcagcggcagcggcagcggcaccagctacagcctgaccatcagccgggtggaggccgaggacgccgccacctactactgccagcagtggaccagcaacccccccaccttcggcggcggcaccaagctgaccgtgctgggtggtggtggctctggaggaggcgggagcgggggtggtggctcaggtggtggaggttcccaggtgcagctgcagcagcccggcgccgagctggtgaagcccggcgccagcgtgaagatgagctgcaaggccagcggctacaccttcaccagctacaacatgcactgggtgaagcagacccccggccggggcctggagtggatcggcgccatctaccccggcaacggcgacaccagctacaaccagaagttcaagggcaaggccaccctgaccgccgacaagagcagcagcaccgcctacatgcagctgagcagcctgaccagcgaggacagcgccgtgtactactgcgcccggagcacctactacggcggcgactggtacttcaacgtgtggggcgccggcaccaccgtgaccgtctcgagtggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 15 - protein, 478 aa, Artificial Sequence
  synthesized
DPVLTQSPSSLSASVGDRVTISCQSSQSVAKNNNLAWFQQKPGQAPKLLIYSASTLAAGVPSRFSGSGSGTDFTLTISSVQPEDFATYYCSARDSGNIQSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSGGGGSGGGGSENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVL

SEQ ID NO: 16 - DNA, 1437 bp, Artificial Sequence
  synthesized
gatccagttctgacacaaagtccatccagcctgtctgcctcagtcggcgacagagtgaccatcagttgccagagctcacagtctgtggctaagaacaacaacttggcgtggttccaacagaaacctggacaggctccgaaattgctgatctattctgcttccacgcttgctgctggtgttccttcccgcttttcaggtagtggtagcgggacagacttcactttgactataagcagcgtgcagcctgaagattttgcgacctactattgttctgctagagacagtggaaatattcagtcctttggggggggaacgaaggtcgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtggaggcggttcaggcggaggtggttccggcggtggcggctcccaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggtggaggcggttcaggcggaggtggttccggcggtggcggctccggtggaggcggctctgaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgtga

SEQ ID NO: 17 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGCGTKLTVLGGGGSGGGGSGGGGSGGGGSEVQLVESGGGLVQPGGSLRLSCTASGFTISTNAMSWVRQAPGKCLEWVGVITGRDITYYASWAKGRFTISRDTSKNTVYLQMNSLRAEDTAVYYCARDGGSSAITSNNIWGQGTLVTVSTGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIKGGGGSGGGGSGRSLVESGGGLVQPGGSLRLSCTASGFTISSYHMQWVRQAPGKCLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGCGTKVEIK

SEQ ID NO: 18 - DNA, 3690 bp, Artificial Sequence
  synthesized
gagatcgtgatgacccagagccccagcaccctgagcgccagcgtgggcgacagggtgatcatcacctgccaggccagcgagagcatcagcagctggctggcctggtaccagcagaagcccggcaaggcccccaagctgctgatctacgaggccagcaagctggccagcggcgtgcccagcaggttcagcggcagcggcagcggcgccgagttcaccctgaccatcagcagcctgcagcccgacgacttcgccacctactactgccagggctacttctacttcatcagcaggacctacgtgaacagcttcggctgcggcaccaagctgaccgtgctgggcggtggcggtagtgggggaggcggttctggcggcggagggtccggcggtggaggatcagaggtgcagctggtggagagcggcggcggcctggtgcagcccggcggcagcctgaggctgagctgcaccgccagcggcttcaccatcagcaccaacgccatgagctgggtgaggcaggcccccggcaagtgcctggagtgggtgggcgtgatcaccggcagggacatcacctactacgccagctgggccaagggcaggttcaccatcagcagggacaccagcaagaacaccgtgtacctgcagatgaacagcctgagggccgaggacaccgccgtgtactactgcgccagggacggcggcagcagcgccatcaccagcaacaacatctggggccagggcaccctggtgaccgtgtcgacaggcggtggagggtccggcggtggtggatcacaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggatccgggaccccggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggttcaggcggaggtggaagtggtggtggcggctctggaggcggcggatctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaaggtggtggcggctctggaggaggagggtccggacggtcgctggtggagtctgggggaggcttggtccagcctggggggtccctgagactctcctgtactgcctctggattcaccatcagtagctaccacatgcagtgggtccggcaggcacctgggaagtgcctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacttggaacctggcgatgctgcaacttactattgtcagtctacctatcttggtactgattatgttggcggtgctttcggctgtgggaccaaggtggagatcaaatga

SEQ ID NO: 19 - protein, 521 aa, Artificial Sequence
  synthesized
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 20 - DNA, 1566 bp, Artificial Sequence
  synthesized
gacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgttcggatccgggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggagggtccggcggtggtggatcctttcttaattcccttttcaaccaagaggttcagatccccttgactgaaagctattgcggcccttgtccgaaaaactggatatgttacaagaataattgttaccaattcttcgacgaaagcaagaactggtatgagagtcaggcgtcttgtatgagtcagaatgccagcctgcttaaggtttattcaaaagaagaccaggatctgcttaagttggtaaagagctaccactggatggggctggtacatatcccaacgaatgggtcatggcagtgggaggacggttctattctgagtccaaatctcctgacgatcatcgaaatgcagaaaggggactgtgccctgtatgcatcatccttcaaggggtacatcgagaactgcagtaccccaaatacctacatttgtatgcaaagaacggttggaggcggtggctcaggcggaggcggctcaggaggtggcggttcaggaggcggcggatctttcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtag

SEQ ID NO: 21 - protein, 1230 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGCGTKLTVLGGGGSGGGGSGGGGSGGGGSEVQLVESGGGLVQPGGSLRLSCTASGFTISTNAMSWVRQAPGKCLEWVGVITGRDITYYASWAKGRFTISRDTSKNTVYLQMNSLRAEDTAVYYCARDGGSSAITSNNIWGQGTLVTVSTGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIKGGGGSGGGGSGRSLVESGGGLVQPGGSLRLSCTASGFTISSYHMQWVRQAPGKCLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGCGTKVEIK

SEQ ID NO: 22 - DNA, 3693 bp, Artificial Sequence
  synthesized
gagatcgtgatgacccagagccccagcaccctgagcgccagcgtgggcgacagggtgatcatcacctgccaggccagcgagagcatcagcagctggctggcctggtaccagcagaagcccggcaaggcccccaagctgctgatctacgaggccagcaagctggccagcggcgtgcccagcaggttcagcggcagcggcagcggcgccgagttcaccctgaccatcagcagcctgcagcccgacgacttcgccacctactactgccagggctacttctacttcatcagcaggacctacgtgaacagcttcggctgcggcaccaagctgaccgtgctgggcggtggcggtagtgggggaggcggttctggcggcggagggtccggcggtggaggatcagaggtgcagctggtggagagcggcggcggcctggtgcagcccggcggcagcctgaggctgagctgcaccgccagcggcttcaccatcagcaccaacgccatgagctgggtgaggcaggcccccggcaagtgcctggagtgggtgggcgtgatcaccggcagggacatcacctactacgccagctgggccaagggcaggttcaccatcagcagggacaccagcaagaacaccgtgtacctgcagatgaacagcctgagggccgaggacaccgccgtgtactactgcgccagggacggcggcagcagcgccatcaccagcaacaacatctggggccagggcaccctggtgaccgtgtcgacaggcggtggagggtccggcggtggtggatcagaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggttcaggcggaggtggaagtggtggtggcggctctggaggcggcggatctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaaggtggtggcggctctggaggaggagggtccggacggtcgctggtggagtctgggggaggcttggtccagcctggggggtccctgagactctcctgtactgcctctggattcaccatcagtagctaccacatgcagtgggtccggcaggcacctgggaagtgcctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacttggaacctggcgatgctgcaacttactattgtcagtctacctatcttggtactgattatgttggcggtgctttcggctgtgggaccaaggtggagatcaaatga

SEQ ID NO: 23 - protein, 522 aa, Artificial Sequence
  synthesized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 24 - DNA, 1569 bp, Artificial Sequence
  synthesized
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggctccagatctgggacggatttcactctgaccatcagcagtctgcagccggaagacttcgcaacttattactgtcagcaacattatactactcctcccacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggagggtccggcggtggtggatcctttcttaattcccttttcaaccaagaggttcagatccccttgactgaaagctattgcggcccttgtccgaaaaactggatatgttacaagaataattgttaccaattcttcgacgaaagcaagaactggtatgagagtcaggcgtcttgtatgagtcagaatgccagcctgcttaaggtttattcaaaagaagaccaggatctgcttaagttggtaaagagctaccactggatggggctggtacatatcccaacgaatgggtcatggcagtgggaggacggttctattctgagtccaaatctcctgacgatcatcgaaatgcagaaaggggactgtgccctgtatgcatcatccttcaaggggtacatcgagaactgcagtaccccaaatacctacatttgtatgcaaagaacggttggaggcggtggctcaggcggaggcggctcaggaggtggcggttcaggaggcggcggatctttcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtag

SEQ ID NO: 25 - protein, 1217 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCSVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 26 - DNA, 3654 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgctccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 27 - protein, 473 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDPVLTQSPSSLSASVGDRVTISCQSSQSVAKNNNLAWFQQKPGQAPKLLIYSASTLAAGVPSRFSGSGSGTDFTLTISSVQPEDFATYYCSARDSGNIQSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 1422 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgggtggaggcggttcaggcggaggtggttccggcggtggcggctccggtggaggcggctctcaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatccgatccagttctgacacaaagtccatccagcctgtctgcctcagtcggcgacagagtgaccatcagttgccagagctcacagtctgtggctaagaacaacaacttggcgtggttccaacagaaacctggacaggctccgaaattgctgatctattctgcttccacgcttgctgctggtgttccttcccgcttttcaggtagtggtagcgggacagacttcactttgactataagcagcgtgcagcctgaagattttgcgacctactattgttctgctagagacagtggaaatattcagtcctttggggggggaacgaaggtcgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 29 - protein, 1533 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCSVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 30 - DNA, 4602 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgctccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 31 - protein, 473 aa, Artificial Sequence; identical to SEQ ID NO: 27
  synthesized

SEQ ID NO: 32 - DNA, 1422 bp, Artificial Sequence; identical to SEQ ID NO: 28
  synthesized

SEQ ID NO: 33 - protein, 1224 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 34 - DNA, 3675 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 35 - protein, 475 aa, Artificial Sequence; identical to SEQ ID NO: 11
  synthesized

SEQ ID NO: 36 - DNA, 1428 bp, Artificial Sequence; identical to SEQ ID NO: 12
  synthesized

SEQ ID NO: 37 - protein, 1540 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 38 - protein, 4623 aa, Artificial Sequence
  synthesized
GAAATCGTCCTTACACAATCTCCTAGCACACTGAGTGTGAGCCCCGGCGAACGCGCGACTTTCTCTTGCAGGGCAAGTCAATCCATAGGGACTAATATACATTGGTATCAACAAAAGCCAGGTAAACCACCCAGGCTTTTGATTAAGTATGCAAGTGAGTCTATTTCCGGTATCCCTGACCGCTTCTCTGGATCAGGCAGTGGCACAGAGTTCACACTCACCATATCTAGTGTGCAATCAGAGGACTTCGCCGTGTATTACTGCCAACAGAATAATAACTGGCCGACTACCTTCGGACCCGGTACAAAGCTGACCGTTTTAGGCGGTGGCGGTAGTGGGGGAGGCGGTTCTGGCGGCGGAGGGTCCGGCGGTGGAGGATCACAAGTACAGTTGCAGCAATCCGGTCCCGGTCTCGTCAAACCGAGTGAGACGCTTAGTATAACGTGTACTGTTTCAGGCTTTAGCCTTACGAACTATGGAGTTCACTGGATTCGGCAGGCACCCGGCAAAGGTTTGGAATGGCTGGGTGTTATTTGGTCAGGTGGAAATACAGACTATAACACCCCCTTTACAAGTCGGTTCACAATTACGAAAGATAATTCCAAAAATCAAGTTTATTTCAAGTTGAGATCCGTCCGCGCGGACGACACTGCGATCTACTATTGTGCGAGGGCACTGACCTACTACGATTACGAATTTGCGTATTGGGGGCAAGGGACTCTTGTAACAGTCTCGAGCGGCGGTGGAGGGTCCGGCGGTGGTGGATCAGAGGTGCAGCTGGTGGAGTCTGGGGGAGGCTTGGTCCAGCCTGGGGGGTCCCTGAGACTCTCCTGTGCAGCCTCTGGATTCACCATCAGTACCAATGCAATGAGCTGGGTCCGCCAGGCTCCAGGGAAGGGGCTGGAGTGGATCGGAGTCATTACTGGTCGTGATATCACATACTACGCGAGCTGGGCGAAAGGCAGATTCACCATCTCCAGAGACAATTCCAAGAACACGCTGTATCTTCAAATGAACAGCCTGAGAGCCGAGGACACGGCTGTGTATTACTGTGCGAGAGACGGTGGTTCTTCTGCTATTACTAGTAACAACATTTGGGGCCAGGGAACCCTGGTCACCGTGTCCTCAGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAAGCCGCGGGGGCACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCGCGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTGGCGGTGGAGGGTCCGGCGGTGGTGGATCCGAGGTGCAGCTGTTGGAGTCTGGGGGAGGCTTGGTACAGCCTGGGGGGTCCCTGAGACTCTCCTGTGCAGCCTCTGGATTCTCCTTCAGTAGCGGGTACGACATGTGCTGGGTCCGCCAGGCTCCAGGGAAGGGGCTGGAGTGGATCGCATGCATTGCTGCTGGTAGTGCTGGTATCACTTACGACGCGAACTGGGCGAAAGGCCGGTTCACCATCTCCAGAGACAATTCCAAGAACACGCTGTATCTGCAAATGAACAGCCTGAGAGCCGAGGACACGGCCGTATATTACTGTGCGAGATCGGCGTTTTCGTTCGACTACGCCATGGACCTCTGGGGCCAGGGAACCCTGGTCACCGTGTCGAGCGGTGGAGGCGGATCTGGCGGAGGTGGTTCCGGCGGTGGCGGCTCCGGTGGAGGCGGCTCTGACATCCAGATGACCCAGTCTCCTTCCACCCTGTCTGCATCTGTAGGAGACAGAGTCACCATCACTTGCCAGGCCAGTCAGAGCATTAGTTCCCACTTAAACTGGTATCAGCAGAAACCAGGGAAAGCCCCTAAGCTCCTGATCTATAAGGCATCCACTCTGGCATCTGGGGTCCCATCAAGGTTCAGCGGCAGTGGATCTGGGACAGAATTTACTCTCACCATCAGCAGCCTGCAGCCTGATGATTTTGCAACTTATTACTGCCAACAGGGTTATAGTTGGGGTAATGTTGATAATGTTTTCGGCGGAGGGACCAAGGTGGAGATCAAAGGCGGTGGAGGGTCCGGCGGTGGTGGCTCCGGACGAGAGGGCCCCGAGCTGTCTCCTGATGACCCAGCAGGCCTCTTGGACTTGCGGCAGGGTATGTTCGCTCAACTTGTGGCTCAGAATGTTCTGCTCATTGATGGACCACTCTCTTGGTATAGTGACCCCGGTCTGGCCGGGGTGAGTCTGACCGGCGGGCTCTCTTATAAAGAGGATACTAAGGAACTGGTCGTAGCAAAAGCGGGCGTTTATTACGTTTTTTTTCAGCTGGAGCTCAGGCGCGTGGTGGCCGGCGAGGGCAGTGGCTCTGTGTCCCTGGCCCTGCACTTACAGCCCTTGAGAAGCGCTGCAGGTGCTGCCGCACTGGCTTTAACTGTTGACCTCCCTCCGGCCTCTTCTGAAGCTAGAAACAGCGCTTTCGGCTTCCAAGGGCGCCTGCTGCACCTGAGCGCAGGCCAGCGCTTAGGTGTGCACCTTCATACAGAGGCCAGGGCCCGACACGCTTGGCAGCTCACACAGGGTGCCACGGTTCTCGGACTTTTCCGCGTTACTCCCGAGATCCCCGCTGGCCTCGGAAGTACTGGTTCTGGGTCTAAACCCGGTTCCGGCGAAGGTAGTACTAAAGGACGAGAAGGGCCAGAGTTAAGTCCAGATGACCCTGCTGGGCTTTTGGACCTGCGGCAGGGCATGTTCGCTCAACTGGTGGCTCAGAACGTGCTGCTGATCGATGGCCCCCTGAGTTGGTACAGCGATCCCGGGCTGGCAGGCGTGTCACTTACAGGGGGCCTCTCTTACAAGGAAGACACCAAGGAGTTAGTGGTCGCTAAGGCTGGCGTGTATTACGTGTTCTTCCAACTGGAGCTGAGAAGGGTTGTGGCAGGAGAGGGTAGCGGCAGCGTGTCTTTAGCCCTTCACTTGCAGCCCCTGAGGTCTGCTGCAGGTGCAGCCGCTCTCGCGCTCACCGTGGATCTCCCCCCAGCCTCATCTGAAGCTAGGAACAGTGCATTTGGCTTTCAGGGACGCTTGCTGCACCTCTCCGCTGGACAGAGGCTGGGCGTGCACCTTCACACAGAGGCCCGTGCCAGGCATGCATGGCAGCTCACTCAGGGGGCAACAGTGCTGGGTCTCTTCCGCGTGACTCCTGAAATACCAGCTGGACTTGGCGGTGGAGGCAGCGGCGGAGGAGGATCTCGTGAGGGGCCAGAACTGTCCCCCGATGACCCAGCCGGACTGCTCGATCTCAGACAGGGCATGTTCGCTCAGCTTGTAGCCCAAAATGTCCTCCTGATTGACGGCCCTTTGAGCTGGTATAGTGATCCCGGCTTGGCCGGGGTATCTCTGACCGGAGGCCTCTCCTACAAGGAAGACACCAAAGAGCTGGTGGTGGCAAAAGCGGGGGTGTATTATGTGTTCTTTCAGCTCGAGCTGCGGAGAGTTGTGGCCGGGGAAGGGTCTGGGAGCGTATCTCTTGCACTTCACCTGCAGCCCCTGCGCAGCGCCGCTGGAGCCGCCGCCCTTGCTCTTACTGTGGATCTGCCTCCTGCTTCCTCAGAAGCACGCAACAGCGCCTTCGGCTTTCAAGGACGTCTCCTGCACTTGTCCGCAGGACAGAGGTTGGGCGTCCATTTACACACTGAGGCACGGGCACGGCACGCTTGGCAGCTTACCCAGGGAGCCACCGTGCTGGGACTCTTTAGAGTGACACCCGAGATCCCCGCTGGCTTGTGA

SEQ ID NO: 39 - protein, 475 aa, Artificial Sequence; identical to SEQ ID NO: 11
  synthesized

SEQ ID NO: 40 - DNA, 1428 bp, Artificial Sequence; identical to SEQ ID NO: 12
  synthesized

SEQ ID NO: 41 - protein, 1165 aa, Artificial Sequence
  synthesized
SGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 42 - DNA, 3687 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 43 - protein, 470 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 1413 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatcagaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 45 - protein, 1217 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 46 - DNA, 3654 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagttggaactgaaaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 47 - protein, 473 aa, Artificial Sequence; identical to SEQ ID NO: 27
  synthesized

SEQ ID NO: 48 - DNA, 1422 bp, Artificial Sequence; identical to SEQ ID NO: 28
  synthesized

SEQ ID NO: 49 - protein, 1533 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 50 - DNA, 4602 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagttggaactgaaaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 51 - protein, 473 aa, Artificial Sequence; identical to SEQ ID NO: 27
  synthesized

SEQ ID NO: 52 - DNA, 1422 bp, Artificial Sequence; identical to SEQ ID NO: 28
  synthesized

SEQ ID NO: 53 - protein, 1234 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEVQLVESGGGLVQPGGSLRLSCAASGFTISTNAMSWVREAPGKCLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 54 - DNA, 3705 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggtggaggcggatctggcggaggtggttccggcggtggcggctccggtggaggcggctctgaggtgcagctggtggagtctgggggaggcttggtccagcctggggggtccctgagactctcctgtgcagcctctggattcaccatcagtaccaatgcaatgagctgggtccgcgaggctccagggaagtgtctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 55 - protein, 739 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 56 - DNA, 2220 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggctgtgggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 57 - protein, 1234 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEVQLVESGGGLVQPGGSLRLSCAASGFTISTNAMSWVREAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 58 - DNA, 3705 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggtggaggcggatctggcggaggtggttccggcggtggcggctccggtggaggcggctctgaggtgcagctggtggagtctgggggaggcttggtccagcctggggggtccctgagactctcctgtgcagcctctggattcaccatcagtaccaatgcaatgagctgggtccgcgaggctccagggaagggcctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 59 - protein, 739 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 60 - DNA, 2220 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggcggcgggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 61 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLQQSGPGLVKPSETLSITCTVSGFSLTNYGVHWIRQAPGKCLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 62 - DNA, 3690 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaatgtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 63 - protein, 734 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 64 - protein, 2205 aa, Artificial Sequence
  synthesized
GAAAATGTATTGACACAGAGCCCCGCCTCCCTCAGTGCCTCACCTGGGGAAAGGGTAACTATCACTTGCTCTGCATCAAGCAGCGTCTCATACATGCATTGGTATCAACAAAAGCCTGGACAGGCCCCCAAGCTCTGGATATACGATACGAGCAAGCTGGCTTCCGGCGTACCTAGCCGCTTCAGTGGTTCCGGCTCAGGCAACGATCACACCCTTACGATTTCCAGTATGGAACCCGAAGATTTTGCAACTTATTATTGTTTCCAGGGGAGCGTGTACCCATTCACTTTCGGGCAGGGGACAAAAGTGACCGTCCTAGGCGGTGGCGGTAGTGGGGGAGGCGGTTCTGGCGGCGGAGGGTCCGGCGGTGGAGGATCACAGGTCACATTGAAGGAATCTGGCCCCGGCCTTGTTCAGCCAGGACAGACCCTTAGCCTCACCTGTGCCTTCAGTGGTTTTTCTCTTAGCACTAGCGGTATGGGGGTCGGCTGGATTCGGCAGCCTCCCGGCAAAGGTCTTGAGTGGTTGGCTCACATTTGGTGGGACGACGACAAACGGTATAATCCTGCCTTGAAAAGTCGGCTGACCATTAGTAAGGATACCTCAAAAAATCAAGTGTACTTGCAAATGAATAGCCTTGACGCCGAGGATACGGCTGTATATTATTGCGCTCGGATGGAACTCTGGTCTTACTACTTTGATTATTGGGGGCAGGGGACTCTCGTCACGGTCTCGAGTGGCGGTGGAGGGTCCGGCGGTGGTGGATCAGAAATCGTCCTTACACAATCTCCTAGCACACTGAGTGTGAGCCCCGGCGAACGCGCGACTTTCTCTTGCAGGGCAAGTCAATCCATAGGGACTAATATACATTGGTATCAACAAAAGCCAGGTAAACCACCCAGGCTTTTGATTAAGTATGCAAGTGAGTCTATTTCCGGTATCCCTGACCGCTTCTCTGGATCAGGCAGTGGCACAGAGTTCACACTCACCATATCTAGTGTGCAATCAGAGGACTTCGCCGTGTATTACTGCCAACAGAATAATAACTGGCCGACTACCTTCGGATGCGGTACAAAGCTGACCGTTTTACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGTGGCGGTGGCGGTAGCGGTGGCGGCGGAAGTGGTGGCGGAGGATCCCAGTCTGCCCTGACTCAGCCTGCCTCCGTGTCTGGGTCTCCTGGACAGTCGATCACCATCTCCTGCACTGGAACCAGCAGTGACGTTGGTGGTTATAACTTTGTCTCCTGGTACCAACAACACCCAGGCAAAGCCCCCAAACTCATGATCTATGATGTCAGTGATCGGCCCTCAGGGGTGTCTGATCGCTTCTCCGGCTCCAAGTCTGGCAACACGGCCTCCCTGATCATCTCTGGCCTCCAGGCTGACGACGAGGCTGATTATTACTGCAGCTCATATGGGAGCAGCAGCACTCATGTGATTTTCGGCGGAGGGACCAAGGTGACCGTCCTAGGTGGAGGCGGTTCAGGCGGAGGTGGTTCCGGCGGTGGCGGCTCCGGTGGAGGCGGCTCTCAGGTGCAATTGCAGGAGTCGGGGGGAGGCCTGGTCAAGCCTGGAGGGTCCCTGAGTCTCTCCTGTGCAGCCTCTGGATTCACCTTTAGTAGTTATTGGATGAGCTGGGTCCGCCAGGCTCCAGGGAAGGGGCTGGAGTGGGTGGCCAACATAAACCGCGATGGAAGTGCGAGTTACTATGTGGACTCTGTGAAGGGCCGATTCACCATCTCCAGAGACGACGCCAAGAACTCACTGTATCTGCAAATGAACAGCCTGAGAGCTGAGGACACGGCTGTGTATTACTGTGCGAGAGATCGTGGGGTGGGCTACTTCGATCTCTGGGGCCGTGGCACCCTGGTCACCGTCTCTAGCTGA

SEQ ID NO: 65 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 66 - protein, 3690 aa, Artificial Sequence
  synthesized
GAAATCGTTATGACGCAGAGTCCCTCCACGCTCTCCGCTAGTGTCGGGGATCGCGTCATTATCACATGCCAGGCCTCCGAGTCAATCAGCAGCTGGCTTGCATGGTATCAACAGAAGCCGGGAAAAGCTCCTAAATTGCTGATCTATGAAGCGTCAAAATTGGCGTCTGGTGTCCCATCTAGGTTCTCCGGCTCTGGGTCTGGTGCGGAATTTACTTTGACAATCTCCAGTCTTCAACCAGACGATTTCGCTACCTACTACTGCCAAGGGTATTTCTATTTTATAAGCCGGACATATGTAAACTCCTTCGGCCAAGGAACAAAGTTGACTGTTCTTGGTGGCGGAGGCAGTGGTGGCGGGGGCAGCGGAGGTGGTGGTTCAGGGGGTGGTGGGAGCGAAGTCCAATTGGTAGAAAGTGGCGGTGGTCTGGTGCAACCTGGTGGATCTCTTCGCCTCTCATGCGCCGCTAGTGGCTTTACTATTTCAACTAATGCGATGAGCTGGGTTCGCCAGGCCCCCGGCAAAGGACTTGAGTGGGTCGGCGTCATCACCGGCAGGGACATTACATACTATGCGAGTTGGGCAAAGGGCAGGTTCACGATTAGCCGCGATACTTCAAAGAATACCGTTTACCTTCAAATGAATAGCTTGAGGGCGGAAGACACAGCTGTGTATTACTGCGCGAGGGATGGAGGTAGTTCCGCCATAACTTCCAACAACATATGGGGACAAGGCACGCTGGTTACTGTCTCGAGTGGCGGTGGAGGGTCCGGCGGTGGTGGATCACAGGTGCAGCTGAAGCAGTCAGGACCTGGCCTAGTGCAGCCCTCACAGAGCCTGTCCATCACCTGCACAGTCTCTGGTTTCTCATTAACTAACTATGGTGTACACTGGGTTCGCCAGTCTCCAGGAAAGTGTCTGGAGTGGCTGGGAGTGATATGGAGTGGTGGAAACACAGACTATAATACACCTTTCACATCCAGACTGAGCATCAACAAGGACAATTCCAAGAGCCAAGTTTTCTTTAAAATGAACAGTCTGCAATCTAATGACACAGCCATATATTACTGTGCCAGAGCCCTCACCTACTATGATTACGAGTTTGCTTACTGGGGCCAAGGGACTCTGGTCACTGTCTCGAGTGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAAGCCGCGGGGGCACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCGCGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTGGCGGTGGAGGGTCCGGCGGTGGTGGATCCGAGGTGCAGCTGTTGGAGTCTGGGGGAGGCTTGGTACAGCCTGGGGGGTCCCTGAGACTCTCCTGTGCAGCCTCTGGATTCTCCTTCAGTAGCGGGTACGACATGTGCTGGGTCCGCCAGGCTCCAGGGAAGGGGCTGGAGTGGATCGCATGCATTGCTGCTGGTAGTGCTGGTATCACTTACGACGCGAACTGGGCGAAAGGCCGGTTCACCATCTCCAGAGACAATTCCAAGAACACGCTGTATCTGCAAATGAACAGCCTGAGAGCCGAGGACACGGCCGTATATTACTGTGCGAGATCGGCGTTTTCGTTCGACTACGCCATGGACCTCTGGGGCCAGGGAACCCTGGTCACCGTGTCGAGCGGTGGAGGCGGATCTGGCGGAGGTGGTTCCGGCGGTGGCGGCTCCGGTGGAGGCGGCTCTGACATCCAGATGACCCAGTCTCCTTCCACCCTGTCTGCATCTGTAGGAGACAGAGTCACCATCACTTGCCAGGCCAGTCAGAGCATTAGTTCCCACTTAAACTGGTATCAGCAGAAACCAGGGAAAGCCCCTAAGCTCCTGATCTATAAGGCATCCACTCTGGCATCTGGGGTCCCATCAAGGTTCAGCGGCAGTGGATCTGGGACAGAATTTACTCTCACCATCAGCAGCCTGCAGCCTGATGATTTTGCAACTTATTACTGCCAACAGGGTTATAGTTGGGGTAATGTTGATAATGTTTTCGGCGGAGGGACCAAGGTGGAGATCAAAGGCGGTGGAGGGTCCGGCGGTGGTGGCTCCGGACGGTCGCTGGTGGAGTCTGGGGGAGGCTTGGTCCAGCCTGGGGGGTCCCTGAGACTCTCCTGTACTGCCTCTGGATTCACCATCAGTAGCTACCACATGCAGTGGGTCCGCCAGGCTCCAGGGAAGGGGCTGGAGTACATCGGAACCATTAGTAGTGGTGGTAATGTATACTACGCAAGCTCCGCTAGAGGCAGATTCACCATCTCCAGACCCTCGTCCAAGAACACGGTGGATCTTCAAATGAACAGCCTGAGAGCCGAGGACACGGCTGTGTATTACTGTGCGAGAGACTCTGGTTATAGTGATCCTATGTGGGGCCAGGGAACCCTGGTCACCGTCTCTTCAGGCGGTGGCGGTAGTGGGGGAGGCGGTTCTGGCGGCGGAGGGTCCGGCGGTGGAGGATCAGACGTTGTGATGACCCAGTCTCCATCTTCCGTGTCTGCATCTGTAGGAGACAGAGTCACCATCACCTGTCAGGCCAGTCAGAACATTAGGACTTACTTATCCTGGTATCAGCAGAAACCAGGGAAAGCCCCTAAGCTCCTGATCTATGCTGCAGCCAATCTGGCATCTGGGGTCCCATCAAGGTTCAGCGGCAGTGGATCTGGGACAGATTTCACTCTCACCATCAGCGACCTGGAGCCTGGCGATGCTGCAACTTACTATTGTCAGTCTACCTATCTTGGTACTGATTATGTTGGCGGTGCTTTCGGCGGAGGGACCAAGGTGGAGATCAAATGA

SEQ ID NO: 67 - protein, 734 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 68 - DNA, 2205 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggttgtgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 69 - protein, 119 aa, Artificial Sequence
  synthesized
EVQLVESGGGLVQPGGSLRLSCKVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSS

SEQ ID NO: 70 - DNA, 357 bp, Artificial Sequence
  synthesized
gaagttcagctggtggaatccggcggaggattggttcaacctggcggctctctgagactgtcctgtaaggtgtctggcttctccctgaccaactacggcgtgcactgggtccgacaggcacctggaaaaggactggaatgggtcggagtgatttggagcggcggcaacaccgactacaacacccctttcaccagccggttcaccatctctcgggacacctccaagaacaccgtgtacctgcagatgaactccctgagagccgaggacaccgccgtgtactattgtgctagagccctgacctactatgactacgagttcgcctattggggccagggaaccctggtcacagtctcctct

SEQ ID NO: 71 - protein, 107 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVL

SEQ ID NO: 72 - DNA, 321 bp, Artificial Sequence
  synthesized
gagatcgtgatgacccagtctccttccacactgtccgcctctgtgggcgacagagtgatcatcacctgtagagccagccagtccatcggcaccaacatccactggtatcagcagaagcctggcaaggcccctaagctgctgatctactacgcctccgagtctatcagcggcatcccctccagattctccggctctggatctggcgctgagtttaccctgacaatctccagcctgcagcctgacgacttcgccacctactactgccagcagaacaacaactggcccaccacctttggccagggcaccaaactgacagttctt

SEQ ID NO: 73 - protein, 119 aa, Artificial Sequence
  synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSS

SEQ ID NO: 74 - DNA, 357 bp, Artificial Sequence
  synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttct

SEQ ID NO: 75 - protein, 107 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELK

SEQ ID NO: 76 - DNA, 321 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctggaattgaaa

SEQ ID NO: 77 - protein, 119 aa, Artificial Sequence; identical to SEQ ID NO: 73
  synthesized

SEQ ID NO: 78 - DNA, 357 bp, Artificial Sequence; identical to SEQ ID NO: 74
  synthesized

SEQ ID NO: 79 - protein, 107 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 80 - DNA, 321 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 81 - protein, 119 aa, Artificial Sequence
  synthesized
QVQLQQSGPGLVKPSETLSITCTVSGFSLTNYGVHWIRQAPGKCLEWLGVIWSGGNTDYNTPFTSRFTITKDNSKNQVYFKLRSVRADDTAIYYCARALTYYDYEFAYWGQGTLVTVSS

SEQ ID NO: 82 - DNA, 357 bp, Artificial Sequence
  synthesized
caagtacagttgcagcaatccggtcccggtctcgtcaaaccgagtgagacgcttagtataacgtgtactgtttcaggctttagccttacgaactatggagttcactggattcggcaggcacccggcaaatgtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagt

SEQ ID NO: 83 - protein, 107 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGCGTKLTVL

SEQ ID NO: 84 - DNA, 321 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggatgcggtacaaagctgaccgtttta

SEQ ID NO: 85 - protein, 120 aa, Artificial Sequence
  synthesized
QVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 86 - DNA, 360 bp, Artificial Sequence
  synthesized
caggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagt

SEQ ID NO: 87 - protein, 106 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVL

SEQ ID NO: 88 - DNA, 318 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtccta

SEQ ID NO: 89 - protein, 120 aa, Artificial Sequence
  synthesized
EVQLVESGGGLVQPGGSLRLSCAASGFTISTNAMSWVRQAPGKCLEWIGVITGRDITYYASWAKGRFTISRDNSKNTLYLQMNSLRAEDTAVYYCARDGGSSAITSNNIWGQGTLVTVST

SEQ ID NO: 90 - DNA, 360 bp, Artificial Sequence
  synthesized
gaggtgcagctggtggagtctgggggaggcttggtccagcctggggggtccctgagactctcctgtgcagcctctggattcaccatcagtaccaatgcaatgagctgggtccgccaggctccagggaagtgcctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcgaca

SEQ ID NO: 91 - protein, 112 aa, Artificial Sequence
  synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGCGTKVEIK

SEQ ID NO: 92 - DNA, 336 bp, Artificial Sequence
  synthesized
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggctgtgggaccaaggtggagatcaaa

SEQ ID NO: 93 - protein, 120 aa, Artificial Sequence
  synthesized
EVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSS

SEQ ID NO: 94 - DNA, 360 bp, Artificial Sequence
  synthesized
gaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagt

SEQ ID NO: 95 - protein, 112 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVL

SEQ ID NO: 96 - DNA, 336 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttctt

SEQ ID NO: 97 - protein, 120 aa, Artificial Sequence
  synthesized
EVQLVESGGGLVQPGGSLRLSCTASGFTISTNAMSWVRQAPGKCLEWVGVITGRDITYYASWAKGRFTISRDTSKNTVYLQMNSLRAEDTAVYYCARDGGSSAITSNNIWGQGTLVTVST

SEQ ID NO: 98 - DNA, 360 bp, Artificial Sequence
  synthesized
gaggtgcagctggtggagagcggcggcggcctggtgcagcccggcggcagcctgaggctgagctgcaccgccagcggcttcaccatcagcaccaacgccatgagctgggtgaggcaggcccccggcaagtgcctggagtgggtgggcgtgatcaccggcagggacatcacctactacgccagctgggccaagggcaggttcaccatcagcagggacaccagcaagaacaccgtgtacctgcagatgaacagcctgagggccgaggacaccgccgtgtactactgcgccagggacggcggcagcagcgccatcaccagcaacaacatctggggccagggcaccctggtgaccgtgtcgaca

SEQ ID NO: 99 - protein, 112 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGCGTKLTVL

SEQ ID NO: 100 - DNA, 336 bp, Artificial Sequence
  synthesized
gagatcgtgatgacccagagccccagcaccctgagcgccagcgtgggcgacagggtgatcatcacctgccaggccagcgagagcatcagcagctggctggcctggtaccagcagaagcccggcaaggcccccaagctgctgatctacgaggccagcaagctggccagcggcgtgcccagcaggttcagcggcagcggcagcggcgccgagttcaccctgaccatcagcagcctgcagcccgacgacttcgccacctactactgccagggctacttctacttcatcagcaggacctacgtgaacagcttcggctgcggcaccaagctgaccgtgctg

SEQ ID NO: 101 - protein, 113 aa, Artificial Sequence
  synthesized
QVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSS

SEQ ID NO: 102 - DNA, 339 bp, Artificial Sequence
  synthesized
caagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatcc

SEQ ID NO: 103 - protein, 110 aa, Artificial Sequence
  synthesized
DPVLTQSPSSLSASVGDRVTISCQSSQSVAKNNNLAWFQQKPGQAPKLLIYSASTLAAGVPSRFSGSGSGTDFTLTISSVQPEDFATYYCSARDSGNIQSFGGGTKVEIK

SEQ ID NO: 104 - DNA, 330 bp, Artificial Sequence
  synthesized
gatccagttctgacacaaagtccatccagcctgtctgcctcagtcggcgacagagtgaccatcagttgccagagctcacagtctgtggctaagaacaacaacttggcgtggttccaacagaaacctggacaggctccgaaattgctgatctattctgcttccacgcttgctgctggtgttccttcccgcttttcaggtagtggtagcgggacagacttcactttgactataagcagcgtgcagcctgaagattttgcgacctactattgttctgctagagacagtggaaatattcagtcctttggggggggaacgaaggtcgaaataaag

SEQ ID NO: 105 - protein, 122 aa, Artificial Sequence
  synthesized
EVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSS

SEQ ID NO: 106 - DNA, 366 bp, Artificial Sequence
  synthesized
gaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagc

SEQ ID NO: 107 - protein, 110 aa, Artificial Sequence
  synthesized
DIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIK

SEQ ID NO: 108 - DNA, 330 bp, Artificial Sequence
  synthesized
gacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaa

SEQ ID NO: 109 - protein, 115 aa, Artificial Sequence
  synthesized
RSLVESGGGLVQPGGSLRLSCTASGFTISSYHMQWVRQAPGKCLEYIGTISSGGNVYYASSARGRFTISRPSSKNTVDLQMNSLRAEDTAVYYCARDSGYSDPMWGQGTLVTVSS

SEQ ID NO: 110 - DNA, 345 bp, Artificial Sequence
  synthesized
cggtcgctggtggagtctgggggaggcttggtccagcctggggggtccctgagactctcctgtactgcctctggattcaccatcagtagctaccacatgcagtgggtccggcaggcacctgggaagtgcctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttca

SEQ ID NO: 111 - protein, 111 aa, Artificial Sequence
  synthesized
DVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGCGTKVEIK

SEQ ID NO: 112 - DNA, 336 bp, Artificial Sequence
  synthesized
gacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacttggaacctggcgatgctgcaacttactattgtcagtctacctatcttggtactgattatgttggcggtgctttcggctgtgggaccaaggtggagatcaaatga

SEQ ID NO: 113 - protein, 562 aa, Artificial Sequence
  synthesized
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 114 - DNA, 1689 bp, Artificial Sequence
  synthesized
cgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 115 - protein, 298 aa, Artificial Sequence
  synthesized
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 116 - DNA, 897 bp, Artificial Sequence
  synthesized
tttcttaattcccttttcaaccaagaggttcagatccccttgactgaaagctattgcggcccttgtccgaaaaactggatatgttacaagaataattgttaccaattcttcgacgaaagcaagaactggtatgagagtcaggcgtcttgtatgagtcagaatgccagcctgcttaaggtttattcaaaagaagaccaggatctgcttaagttggtaaagagctaccactggatggggctggtacatatcccaacgaatgggtcatggcagtgggaggacggttctattctgagtccaaatctcctgacgatcatcgaaatgcagaaaggggactgtgccctgtatgcatcatccttcaaggggtacatcgagaactgcagtaccccaaatacctacatttgtatgcaaagaacggttggaggcggtggctcaggcggaggcggctcaggaggtggcggttcaggaggcggcggatctttcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtag

SEQ ID NO: 117 - protein, 1247 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGCGTKLTVLGSTGSGSKPGSGEGSTKGEVQLVESGGGLVQPGGSLRLSCTASGFTISTNAMSWVRQAPGKCLEWVGVITGRDITYYASWAKGRFTISRDTSKNTVYLQMNSLRAEDTAVYYCARDGGSSAITSNNIWGQGTLVTVSTGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIKGGGGSGGGGSGRSLVESGGGLVQPGGSLRLSCTASGFTISSYHMQWVRQAPGKCLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGCGTKVEIK

SEQ ID NO: 118 - DNA, 3744 bp, Artificial Sequence
  synthesized
gagatcgtgatgacacaatctccatctacgctctccgcctcagtgggcgatagagtaattattacttgtcaagcctcagagagcatttcatcatggctcgcctggtatcagcaaaagcctgggaaggcccccaaacttctcatctatgaagcatcaaagctggcctctggggttccgtctcgcttctccgggtccggcagtggtgcagagtttacgttgactatatcttctttgcaacctgacgatttcgcaacatattattgccagggatacttttattttatttcccgaacatatgttaactcttttgggtgcgggaccaaactcactgtgctggggtctaccggtagtggttctaagcctggttcaggcgaaggcagtacgaaaggggaagtgcaactggtcgaaagcggtggagggcttgttcaacctggaggaagcctccgcttgtcctgcacggctagcggctttacaataagtacgaacgccatgagctgggtccggcaggctccaggtaagtgtctcgaatgggtgggggtcataacaggcagggacattacctactacgccagttgggccaagggtcgatttaccatttctagagatacatccaagaacacggtgtacctccagatgaattctcttagggcggaagacacagcagtatactactgcgcgcgagatggcgggagcagtgcgatcacatccaacaacatctggggtcagggcactcttgtcacggtgtcgactggtggtgggggtagtggcggcggaggtagctttcttaattcccttttcaaccaagaggttcagatccccttgactgaaagctattgcggcccttgtccgaaaaactggatatgttacaagaataattgttaccaattcttcgacgaaagcaagaactggtatgagagtcaggcgtcttgtatgagtcagaatgccagcctgcttaaggtttattcaaaagaagaccaggatctgcttaagttggtaaagagctaccactggatggggctggtacatatcccaacgaatgggtcatggcagtgggaggacggttctattctgagtccaaatctcctgacgatcatcgaaatgcagaaaggggactgtgccctgtatgcatcatccttcaaggggtacatcgagaactgcagtaccccaaatacctacatttgtatgcaaagaacggttgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggttcaggcggaggtggaagtggtggtggcggctctggaggcggcggatctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaaggtggtggcggctctggaggaggagggtccggacggtcgctggtggagtctgggggaggcttggtccagcctggggggtccctgagactctcctgtactgcctctggattcaccatcagtagctaccacatgcagtgggtccggcaggcacctgggaagtgcctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacttggaacctggcgatgctgcaacttactattgtcagtctacctatcttggtactgattatgttggcggtgctttcggctgtgggaccaaggtggagatcaaatga

SEQ ID NO: 119 - protein, 505 aa, Artificial Sequence
  synthesized
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGSTGSGSKPGSGEGSTKGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - DNA, 1518 bp, Artificial Sequence
  synthesized
gacatccagatgacccagtctccatcctccctgtctgcatctgtaggagacagagtcaccatcacttgccaggcgagtcaggacatcagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgcaacatatttctgtcaacactttgatcatctcccgctcgctttcggcggagggaccaaggtggaaattaaaggaagtactggttctgggtctaaacccggttccggcgaaggtagtactaaaggacaggtgcagctgcaggagtcgggcccaggactggtgaagccttcggagaccctgtccctcacctgcactgtctctggtggctccgtcagcagtggtgattactactggacctggatccggcagtccccagggaagggactggagtggattggacacatctattacagtgggaacaccaattataacccctccctcaagagccgactcaccatatcaattgacacgtccaagactcagttctccctgaagctgagttctgtgaccgctgcggacacggccatttattactgtgtgcgagatcgagtgactggtgcttttgatatctggggccaagggacaatggtcaccgtctcgagcggtggcggcggctccgggggtggcggatcaggtggtggaggctctttcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 121 - protein, 120 aa, Artificial Sequence
  synthesized
QVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSS

SEQ ID NO: 122 - DNA, 360 bp, Artificial Sequence
  synthesized
caggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagc

SEQ ID NO: 123 - protein, 1247 aa, Artificial Sequence; identical to SEQ ID NO: 117
  synthesized

SEQ ID NO: 124 - DNA, 3744 bp, Artificial Sequence; identical to SEQ ID NO: 118
  synthesized

SEQ ID NO: 125 - protein, 505 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIKGSTGSGSKPGSGEGSTKGQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - DNA, 1518 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaagggaagtactggttctgggtctaaacccggttccggcgaaggtagtactaaaggacaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtgtcctctggtggcggcggctccgggggtggcggatcaggtggtggaggatccttcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 127 - protein, 1563 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGCGTKLTVLGSTGSGSKPGSGEGSTKGEVQLVESGGGLVQPGGSLRLSCTASGFTISTNAMSWVRQAPGKCLEWVGVITGRDITYYASWAKGRFTISRDTSKNTVYLQMNSLRAEDTAVYYCARDGGSSAITSNNIWGQGTLVTVSTGGGGSGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKCLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGCGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 128 - DNA, 4692 bp, Artificial Sequence
  synthesized
gagatcgtgatgacacaatctccatctacgctctccgcctcagtgggcgatagagtaattattacttgtcaagcctcagagagcatttcatcatggctcgcctggtatcagcaaaagcctgggaaggcccccaaacttctcatctatgaagcatcaaagctggcctctggggttccgtctcgcttctccgggtccggcagtggtgcagagtttacgttgactatatcttctttgcaacctgacgatttcgcaacatattattgccagggatacttttattttatttcccgaacatatgttaactcttttgggtgcgggaccaaactcactgtgctggggtctaccggtagtggttctaagcctggttcaggcgaaggcagtacgaaaggggaagtgcaactggtcgaaagcggtggagggcttgttcaacctggaggaagcctccgcttgtcctgcacggctagcggctttacaataagtacgaacgccatgagctgggtccggcaggctccaggtaagtgtctcgaatgggtgggggtcataacaggcagggacattacctactacgccagttgggccaagggtcgatttaccatttctagagatacatccaagaacacggtgtacctccagatgaattctcttagggcggaagacacagcagtatactactgcgcgcgagatggcgggagcagtgcgatcacatccaacaacatctggggtcagggcactcttgtcacggtgtcgactggtggtgggggtagtggcggcggaggtagctttcttaattcccttttcaaccaagaggttcagatccccttgactgaaagctattgcggcccttgtccgaaaaactggatatgttacaagaataattgttaccaattcttcgacgaaagcaagaactggtatgagagtcaggcgtcttgtatgagtcagaatgccagcctgcttaaggtttattcaaaagaagaccaggatctgcttaagttggtaaagagctaccactggatggggctggtacatatcccaacgaatgggtcatggcagtgggaggacggttctattctgagtccaaatctcctgacgatcatcgaaatgcagaaaggggactgtgccctgtatgcatcatccttcaaggggtacatcgagaactgcagtaccccaaatacctacatttgtatgcaaagaacggttgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaagtgcctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagcggtggaggcggttcaggcggaggtggaagtggtggtggcggctctggaggcggcggatctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggctgcgggaccaaggtggagatcaaaggtggtggcggctctggaggaggagggtccggacgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 129 - protein, 505 aa, Artificial Sequence; identical to SEQ ID NO: 125
  synthesized

SEQ ID NO: 130 - DNA, 1518 bp, Artificial Sequence; identical to SEQ ID NO: 126
  synthesized

SEQ ID NO: 131 - protein, 106 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKLEIK

SEQ ID NO: 132 - DNA, 318 bp, Artificial Sequence
  Synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaattggagataaag

(19) United States
(12) Patent Application Publication
(10) Pub. No.: US 2023/0002488 A1
(43) Pub. Date: Jan. 5, 2023

(54) GUIDANCE AND NAVIGATION CONTROL PROTEINS AND METHOD OF MAKING AND USING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Dennis R. Goulet, Redmond, WA (US); Soumili Chatterjee, Bothell, WA (US); Tsung-I Tsai, Redmond, WA (US); Blair Renshaw, Renton, WA (US); Andrew Waight, Kenmore, WA (US); Nga Sze Amanda Mak, Mukilteo, WA (US); Yi ZHU, Redmond, WA (US)

(21) Appl. No.: 17/773,240

(22) PCT Filed: Nov. 5, 2020
(86) PCT No.: PCT/US2020/059230

§ 371 Date: Apr. 29, 2022

Related U.S. Application Data
(60) Provisional application No. 62/931,307, filed on Nov. 6, 2019.
(60) Provisional application No. 62/984,731, filed on Mar. 3, 2020.
(60) Provisional application No. 62/991,042, filed on Mar. 17, 2020.

(51) Int. Cl. C07K 16/28; A61P 35/00; C07K 16/32; A61K 47/68

(52) U.S. Cl. CPC C07K 16/2809; A61P 35/00; C07K 16/32; C07K 16/2863; C07K 16/2878; A61K 47/6889; A61K 2039/505

(57) ABSTRACT

The application provides a multi-specific antibody-like protein having a N-terminal and a C-terminal, comprising in tandem from the N-terminal to the C-terminal, a first binding domain (D1) at the N-terminal, a second binding domain (D2) comprising a light chain moiety, a Fc region, a third binding domain (D3), and a fourth binding domain (D4) at the C-terminal, wherein the light chain moiety comprises a fifth binding domain (D5) covalently attached to the C-terminal, a sixth binding domain (D6) covalently attached to the N-terminal, or both, and wherein the D1, D2, D3, D4, D5 and D6 each has a binding specificity against a tumor antigen, an immune signaling antigen, or a combination thereof.

### CROSS REFERENCE TO RELATED APPLICATIONS

This application claims the benefit of the filing date of U.S. Provisional Application Ser. No. 62/931,307 filed Nov. 6, 2019, U.S. Provisional Application Ser. No. 62/984,731 filed Mar. 3, 2020, and U.S. Provisional Application Ser. No. 62/991,042 filed Mar. 17, 2020 under 35 U.S.C. 119(e), the entire disclosures of which are incorporated by reference herein.

### TECHNICAL FIELD

The present application relates to the technical field of multi-specific antibody for immunotherapy and more particularly relates to making and using Guidance and Navigation Control (GNC) antibodies with multiple binding activities against surface molecules of immune cells and tumor cells.

### BACKGROUND

Cancer develop by gaining mutations that enable the cancer cells to transform, proliferate, and metastasize while escaping from the immune surveillance and response. Antibody therapy for treating cancer recruits multiple distinct mechanisms. For example, monoclonal antibodies targeting growth receptors (EGFR, HER2, etc.) that are overexpressed on tumor cells can be used to block tumor cell proliferation. Using antibodies to block inhibitory T cell checkpoint signals (anti-PDL1, anti-PD1, anti-CTLA4) is a strategy to prevent tumor cells from weakening the immune response that would otherwise seek to control their growth. Another therapeutic strategy is to inhibit angiogenesis (e.g., anti-VEGF), where the reduced access to oxygen and nutrients slows the growth of tumor cells. Monoclonal antibodies and antibody-drug conjugates (ADCs) are initially effective at controlling tumors. However, cancer resistance to antibody therapy often occurs through escape mechanisms, such as ectodomain shedding, receptor downregulation and receptor mutation (Miller et al. Clin Cancer Res. 2017; Reslan et al. Mabs. 2009; Loganzo et al. Mol Cancer Ther. 2016). For example, resistance to anti-HER2 mAb trastuzumab may occur through ectodomain shedding of HER2 or through occlusion of the trastuzumab epitope on HER2 (Fiszman and Jasnis. International Journal of Breast Cancer, 2011).

Combinational therapies combining multiple therapeutic mechanisms including that of chemotherapy, radiation therapy and antibody therapy have become a mainstream therapeutic strategy. In this context, multi-specific antibodies combine different antibody therapies and mechanisms into a single agent (Boumandi and de Sauvage. Nat Rev Drug Discov. 2020).

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides guidance and navigation control (GNC) proteins that can simultaneously bind effector cells and target cells. The GNC protein may be a monomer or a dimer of the monomer. The GNC protein may be an antibody or an antibody-like protein. The GNC protein may have at least 5 or at least 6 binding domains.

In one embodiment, the application provides multi-specific antibody-like proteins having a N-terminal and a C-terminal, comprising in tandem from the N-terminal to the C-terminal, a first binding domain (D1) at the N-terminal, a second binding domain (D2) comprising a light chain moiety, a Fc region, a third binding domain (D3), and a fourth binding domain (D4) at the C-terminal. The light chain moiety comprises a fifth binding domain (D5) covalently attached to the C-terminal, a sixth binding domain (D6) covalently attached to the N-terminal, or both. The D1, D2, D3, D4, D5 and D6 each has a binding specificity to a tumor antigen, an immune signaling antigen, or a combination thereof.

The tumor antigen may be a tissue antigen, a neoantigen, a tumor-specific antigen (TSA), a tumor-associated antigen (TAA), or a combination there.

The D2 may include C_{H1}. In one embodiment, the light chain moiety in the D2 may include C_{L}. In one embodiment, the light chain moiety may include Cκ/Cλ.

The D2 may include a dimer.

In on embodiment, the D2 may include a Fab region. In one embodiment, the Fab region may have a disulfide bond between V_{L} and V_{H}. In one embodiment, the D2 may include a V_{L} and a V_{H}.

In one embodiment, the D2 may include a receptor. In one embodiment, the receptor may be NKG2D. In one embodiment, the D2 may include NKG2D connected to C_{H1} and C_{L}. In one embodiment, the D2 may have an amino acid sequence having at least 50%, 60%, 70%, 75%, 80%, 85%, 90%, 95%, 98%, or 99% of sequence identity to SEQ ID NO. 155 and 116.

The D2 may be connected to the Fc region through a hinge.

The Fc region may include null mutation, which may have the effect to reduce or eliminate effector functions. In one embodiment, the Fc region may be wild-type Fc. In one embodiment, the Fc region may include LALAKA mutations for null Fc. In one embodiment, the LALAKA mutations for null Fc may include L234A/L235A/K322A (Eu numbering) mutations. In one embodiment, the Fc region may include G237A (Eu numbering) mutation. In one embodiment, the Fc region may include N297A (Eu numbering) mutation. In one embodiment, the Fc region may include a glycosylated Fc. In one embodiment, the Fc region may be an aglycosylated Fc to reduce effector function.

In one embodiment, the application may provide a multi-specific antibody-like protein having a N-terminal and a C-terminal, comprising in tandem from the N-terminal to the C-terminal, a first binding domain (D1) at the N-terminal, a second binding domain (D2) comprising a dimer connected to C_{L} and C_{H1}, a Fc region comprising CH2 and CH3, wherein the CH2 is connected to the C_{H1} through a hinge, a third binding domain (D3), and a fourth binding domain (D4) at the C-terminal. The light chain moiety may have a fifth binding domain (D5) covalently attached to the C-terminal, a sixth binding domain (D6) covalently attached to the N-terminal, or both. The D1, D2, D3, D4, D5 and D6 each may have a binding specificity to a tumor antigen, an immune signaling antigen, or a combination thereof.

The dimer in the D2 may include V_{L} and V_{H} pair connected to C_{L} and C_{H} respectively, in which case the D2 domain may be a Fab region, and the GNC protein may be a multi-specific antibody monomer or a multi-specific antibody.

In one embodiment, the multi-specific antibody-like protein may be either penta-specific or hexa-specific.

In one embodiment, the light chain moiety in the D2 may have a fifth binding domain (D5) covalently attached to the C-terminal, and the multi-specific antibody-like protein is penta-specific. In one embodiment, the light chain moiety may have a sixth binding domain (D6) covalently attached to the N-terminal, and the multi-specific antibody-like protein is penta-specific. In one embodiment, the light chain moiety may have a fifth binding domain (D5) covalently attached to the C-terminal and a sixth binding domain (D6) covalently attached to the N-terminal simultaneously, which makes the multi-specific antibody-like protein to be hexa-specific.

The D1, D2, D3, D4, D5, and D6 may be independently a scFv domain, a receptor, or a ligand.

The scFv domain may have the configuration of V_{L}V_{H} or V_{H}V_{L} from the N terminal to the C terminal. In one embodiment, the scFv domain may include R19S (Kabat) mutation. In one embodiment, the scFv domain may include a disulphide bond between V_{L} and V_{H}. In one embodiment, the disulfide bond may be between vL100 and vH44 (Kabat) of the scFv domain. In one embodiment, the scFv domain may have an amino acid sequence having at least 50%, 60%, 70%, 75%, 80%, 85%, 90%, 95%, 98%, or 99% of sequence identity to SEQ ID NO. 72-112.

In one embodiment, the D1, D2, D3, D4, D5, and D6 may all be scFv domains.

In one embodiment, the D1, D2, D3, D4, D5 and D6 each may be independently a receptor or a ligand. In one embodiment, at least one, two, three, four, or five of the D1, D2, D3, D4, D5, and D6 may be a receptor or a ligand. In one embodiment, the D1, D2 D3, D4, D5, and D6 may all be receptors or ligands. In one embodiment, the D4, D5 or D6 may be a receptor or a ligand. In one embodiment, the receptor or a ligand may have an amino acid sequence having at least 50%, 60%, 70%, 75%, 80%, 85%, 90%, 95%, 98%, or 99% of sequence identity to SEQ ID NO. 113-116.

In one embodiment, the D2 has a binding specificity to CD3 or a tumor associated antigen (TAA).

In one embodiment, the D1, D2, D3, D4, D5, and D6 independently has a binding specificity to an antigen selected from a receptor on a T cell, an immune checkpoint receptor, a co-stimulation receptor, a receptor of a lymphocyte or a myeloid cell, a tumor associated antigen (TAA), a tissue antigen, a neoantigen, a tumor-specific antigen (TSA), a glycoprotein, or a combination thereof.

In one embodiment, the binding domain for the receptor on the T cell may be adjacent to the binding domain for the tumor associated antigen (TAA). In one embodiment, the binding domain for the receptor on the T cell is adjacent to the binding domain for the receptor of a lymphocyte or a myeloid cell.

In one embodiment, the receptor on the T cell may be CD3, T cell receptor, or a complex thereof. In one embodiment, the immune checkpoint receptor may be PD-L1, PD-1, TIGIT, TIM-3, LAG-3, CTLA4, BTLA, VISTA, PDL2, CD160, LOX-1, siglec-15, CD47, SIRPα, or a combination thereof. In one embodiment, the co-stimulating receptor may be 4-1BB, CD28, OX40, GITR, CD40, ICOS, CD27, CD30, CD226, or a combination thereof. In one embodiment, the tumor associated antigen (TAA) may be EGFR, HER2, HER3, HER4, EGRFVIII, CD19, claudin 18.2, BCMA, CD20, CD33, CD123, CD22, CD30, ROR1, CEA, cMET, LMP1, LMP2A, Mesothelin, PSMA, EpCAM, glypican-3, gpA33, GD2, TACI, TROP2, NKG2D ligands, PD-L1, or a combination thereof.

In one embodiment, the D1, D2, D3, D4, D5 and D6 each independently may have a binding specificity to an antigen selected from EGFR, HER2, HER3, EGFRvIII, ROR1, CD3, CD28, CEA, LMP1, LMP2A, Mesothelin, PSMA, EpCAM, glypican-3, gpA33, GD2, TROP2, NKG2D ligands, BCMA, CD19, CD20, CD33, CD123, CD22, CD30, PD-L1, PD1, OX40, 4-1BB, GITR, TIGIT, TIM-3, LAG-3, CTLA4, CD40, VISTA, ICOS, BTLA, LIGHT, HVEM, CSF1R, CD73, and CD39, CLDN18.2, CSF1R, and wherein the Fc region comprises a human IgG Fc region.

In one embodiment, the D2 and D5 each independently has a binding specificity to a tumor associated antigen, a neoantigen, or a tumor-specific antigen (TSA).

In one embodiment, the D1 has a binding specificity to CD3, CD20, EGFR, or their derivative thereof. In one embodiment, the D2 has the binding specificity to EGFR, CD3, HER2, MSLN, NKG2D ligands, or their derivative thereof. In one embodiment, the D3 has a binding specificity to PD-L1. In one embodiment, the D4 may include a 4-1BBL trimer or has a binding specificity to 4-1BB or its derivative thereof. In one embodiment, the D5 has a binding specificity to HER3, CD19, NKG2D ligands, or their derivative thereof. In one embodiment, the D6 has a binding specificity to CD19.

In one embodiment, the multi-specific antibody-like protein is penta-specific, and wherein the D1 has a binding specificity to CD3, D2 has a binding specificity to EGFR, D3 has a binding specificity to PD-L1, D4 has a binding specificity to 4-1BB, and D5 has a binding specificity to HER3. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 1-8.

In one embodiment, the multi-specific antibody-like protein is penta-specific, and wherein the D1 has a binding specificity to CD20, D2 has a binding specificity to CD3, D3 has a binding specificity to PD-L1, D4 has a binding specificity to 4-1BB, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 9-12.

In one embodiment, the multi-specific antibody-like protein is penta-specific, and wherein the D1 has a binding specificity to CD20, D2 has a binding specificity to CD3, D3 has a binding specificity to PD-L1, D4 has a binding specificity to 4-1BB, and D5 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 13-16.

In one embodiment, the multi-specific antibody-like protein is penta-specific, and wherein the D1 has a binding specificity to CD3, D2 has a binding specificity to MSLN, D3 has a binding specificity to PD-L1, D4 has a binding specificity to 4-1BB, and D5 has a binding specificity to NKG2D ligands. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 17-20.

In one embodiment, the multi-specific antibody-like protein is penta-specific, and wherein the D1 has a binding specificity to CD3, D2 has a binding specificity to HER2, D3 has a binding specificity to PD-L1, D4 has a binding specificity to 4-1BB, and D5 has a binding specificity to NKG2D ligands. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 21-24.

In one embodiment, the multi-specific antibody-like protein is penta-specific, and wherein the D1 has a binding specificity to EGFR, D2 has a binding specificity to CD3, D3 has a binding specificity to PD-L1, D4 has a binding specificity to 4-1BB, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 25-28.

In one embodiment, the multi-specific antibody-like protein is penta-specific, and wherein the D1 has a binding specificity to EGFR, D2 has a binding specificity to CD3, D3 has a binding specificity to PD-L1, D4 comprises 4-1BB ligand trimer, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 29-32.

In one embodiment, the multi-specific antibody-like protein is penta-specific, and wherein the D1 has a binding specificity to EGFR, D2 has a binding specificity to CD3, D3 has a binding specificity to PD-L1, D4 has a binding specificity to 4-1BB, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 33-36.

In one embodiment, the multi-specific antibody-like protein is penta-specific, and wherein the D1 has a binding specificity to EGFR, D2 has a binding specificity to CD3, D3 has a binding specificity to PD-L1, D4 comprises 4-1BB ligand trimer, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 37-40.

In one embodiment, the multi-specific antibody-like protein is penta-specific, and wherein the D1 has a binding specificity to CD3, D2 has a binding specificity to EGFR, D3 has a binding specificity to PD-L1, D4 has a binding specificity to 4-1BB, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 41-44.

In one embodiment, the multi-specific antibody-like protein is penta-specific, and wherein the D1 has a binding specificity to EGFR, D2 has a binding specificity to CD3, D3 has a binding specificity to PD-L1, D4 has a binding specificity to 4-1BB, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 45-48.

In one embodiment, the multi-specific antibody-like protein is penta-specific, and wherein the D1 has a binding specificity to EGFR, D2 has a binding specificity to CD3, D3 has a binding specificity to PD-L1, D4 comprises 4-1BB ligand trimer, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 49-52.

In one embodiment, the multi-specific antibody-like protein is penta-specific, and wherein the D1 has a binding specificity to CD3, D2 comprises NKG2D, D3 has a binding specificity to PD-L1, D4 has a binding specificity to 4-1BB, D6 has the binding specificity to EGFR. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 117-120.

In one embodiment, the multi-specific antibody-like protein is penta-specific, and wherein the D1 has a binding specificity to CD3, D2 comprises NKG2D, D3 has a binding specificity to PD-L1, D4 has a binding specificity to 4-1BB, D6 has the binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 123-126.

In one embodiment, the multi-specific antibody-like protein is penta-specific, and wherein the D1 has a binding specificity to CD3, D2 comprises NKG2D, D3 has a binding specificity to PD-L1, D4 comprises 4-1BB ligand trimer, D6 has the binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 127-130.

In one embodiment, the multi-specific antibody-like protein is hexa-specific, and wherein the D1 has a binding specificity to EGFR, D2 has a binding specificity to CD3, D3 has a binding specificity to PD-L1, D4 has a binding specificity to 4-1BB, D5 has the binding specificity to HER3, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 53-60.

In one embodiment, the multi-specific antibody-like protein is hexa-specific, and wherein the D1 has a binding specificity to CD3, D2 has a binding specificity to EGFR, D3 has a binding specificity to PD-L1, D4 has a binding specificity to 4-1BB, D5 has the binding specificity to HER3, and D6 has a binding specificity to CD19. In one embodiment, the multi-specific antibody-like protein has an amino acid sequence having at least 50%, 60%, 70%, 75%, 80%, 85%, 90%, 95%, 98%, or 99% of sequence identity to SEQ ID NO. 61-68.

In one embodiment, the D1, D3, D4, D5 or D6 may include a (GₓS_{y})ₙ linker. n may be an integer from 1 to 10. x may be an integer from 1 to 10. y may be an integer from 1 to 10.

In one embodiment, the application may provide a guidance navigation control (GNC) protein that include the multi-specific antibody-like protein as described thereof. In one embodiment, such GNC protein may be a dimer of the multi-specific antibody-like protein as described herein.

In one aspect, the application provides isolated nucleic acid sequences encoding an amino acid sequence of the multi-specific antibody-like protein or its fragments or derivatives as disclosed herein.

In one aspect, the application provides expression vector including the isolated nucleic acid sequence as described herein.

In one aspect, the application provides host cells comprising the isolated nucleic acid sequence as disclosed thereof. In one embodiment, the host cell may be a prokaryotic cell or a eukaryotic cell.

In one aspect, the application provides methods for producing GNC proteins as disclosed herein. In one embodiment, the method for producing a multi-specific antibody or monomer as disclosed herein may include the steps of culturing a host cell comprising an isolated nucleic acid sequence such that the DNA sequence encoding the multi-specific antibody or monomer is expressed, and purifying said multi-specific antibody, wherein the isolated nucleic acid sequence encodes an amino acid of the multi-specific antibody-like protein as disclosed herein.

In one aspect, the application provides immuno-conjugate comprising a cytotoxic agent or an imaging agent linked to the GNC protein such as a multi-specific antibody-like protein or a multi-specific antibody disclosed herein through a linker. The linker may include a covalent bond such as an ester bond, an ether bond, an amid bond, a disulphide bond, an imide bond, a sulfone bond, a phosphate bond, a phosphorus ester bond, a peptide bond, a hydrophobic poly(ethylene glycol) linker, or a combination thereof.

In one embodiment, the cytotoxic agent or the imaging agent may be a chemotherapeutic agent, a growth inhibitory agent, a cytotoxic agent from class of calicheamicin, an antimitotic agent, a toxin, a radioactive isotope, a toxin, a therapeutic agent, or a combination thereof.

In one aspect, the application provides pharmaceutical composition for treating, preventing or controlling conditions such as cancer, autoimmune diseases, or infectious diseases. In one embodiment, the composition may include a pharmaceutically acceptable carrier and a GNC protein such as a multi-specific antibody or a multi-specific antibody-like protein, their immuno-conjugate or their fragment thereof.

In one embodiment, the pharmaceutical composition may further include a therapeutic agent selected from a radioisotope, radionuclide, a toxin, a chemotherapeutic agent or a combination thereof.

In one aspect, the application provides methods for treating, preventing or controlling conditions such as cancer, autoimmune diseases, or an infectious disease. In one embodiment, the method includes the steps of administering a pharmaceutical composition comprising a purified multi-specific antibody, the multi-specific antibody-like protein or its fragments, as disclosed herein.

In one aspect, the application provides methods for treating a human subject with a cancer, an autoimmune disease, or an infection. In one embodiment, the method includes the step of administering to the subject an effective amount of the GNC protein such as the purified multi-specific antibody or the multi-specific antibody-like protein or their fragments as disclosed herein.

In one embodiment, the method may further include the step of co-administering an effective amount of a therapeutic agent, wherein the therapeutic agent comprises an antibody, a chemotherapy agent, an enzyme, an anti-estrogen agent, a receptor tyrosine kinase inhibitor, a kinase inhibitor, a cell cycle inhibitor, a check point inhibitor, a DNA, RNA or protein synthesis inhibitor, a RAS inhibitor, an inhibitor of PD1, PD-L1, CTLA4, 4-1BB, OX40, GITR, ICOS, LIGHT, TIM3, LAG3, TIGIT, CD40, CD27, HVEM, BTLA, VISTA, B7H4, CSF1R, NKG2D, CD73, or a combination thereof.

In one aspect, the application provides a solution comprising an effective concentration of the GNC protein such as the multi-specific antibody or the multi-specific antibody-like protein or their fragments thereof. In one embodiment, the solution may be blood plasma in a human subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows a schematic configuration of antigen binding domains in (A) a penta-GNC antibody and (B) a hexa-GNC antibody: the variable regions (replaceable by a receptor or ligand) of Fab in black (D2); both the constant region of Fab and the Fc region in white; additional scFv antigen binding domains in shaded boxes (each replaceable by a receptor-ligand binding); a heavy chain monomer linking D1 to its N-terminus and D3 and D4 tandemly to its C-terminus through D4; and a light chain moiety monomer linking D5 and/or D6 to its N- and C-terminus;

FIG. 2 shows that the penta-GNC antibody (SI-1P1) exerts maximized T-cell activation in the presence of human pancreatic cancer cells (BxPC3) that express high levels of EGFR and low levels of HER3, with similar potency to that of tetra-GNC antibodies targeting either HER3 (Tetra) or no tumor antigen (Tetra, FITC), as well as the bispecific antibody only targeting tumor antigens (BI);

FIG. 3 shows the high potency of SI-1P1 in TDCC assay by using cancer cell lines expressing high levels of EGFR and low levels of HER3 in (A) human breast cancer cells (MDA-MB-231) and (B) human cervical cancer cells (HeLa), and control antibodies including a comparable tetra-GNC antibody lacking the binding to HER3, a tetra-GNC control antibody lacking the binding to both tumor antigens, and a bispecific antibody targeting tumor antigens only;

FIG. 4 shows the effect of having NKG2D receptor as a binding domain for the GNC antibodies: (A) the potency of SI-49P3 mediated T cell activation using human pancreatic cancer cells (BxPC3); and (B) high potency of SI-49P1 in TDCC assay using human breast cancer cells (MDA-MB-231) that express tumor antigens (MICA and mesothelin) other than EGFR and HER3, and two control antibodies: a tetra-GNC antibody lacking NKG2D and a tri-GNC antibody lacking binding specificities to both PD-L1 and 4-1BB;

FIG. 5 shows that the 4-1BBL-trimer-Fc fusion protein mediates robust activation of 4-1BB signaling as measured by a reporter bioassay using Jurkat cells, when compared to other molecules containing monomeric 4-1BB ligand, monomeric Fc, or an anti-4-1BB scFv;

FIG. 6 shows the Octet binding analysis of penta-GNC antibodies comprising humanized anti-huEGFR domains, indicating that variants of humanized EGFR binding domains (H1, H4, or H7) retain tight binding to human EGFR with little positional effect as either a scFv domain in SI-55P3, SI-79P2, SI55P9, and SI-79P3 or Fab in SI-77P1;

FIG. 7 shows the potency of penta-GNC antibodies in TDCC assay using human pancreatic cancer cells (BxPC3) as targeted cells and the EC50 values: (A) SI-1P1, 0.2814 pM; (B) SI-55P9, 0.4871 pM; and (C) SI-55P10, 0.7358 pM;

FIG. 8 shows the potency of penta-GNC proteins containing NKG2D at position D2 in TDCC assay using human breast cancer cells (MDA-MB-231, with MICA expression) as targeted cells, with the resulting the EC50 values: SI-49P6, 0.7366 pM and SI-49P7, 0.1094 pM;

FIG. 9 shows that a tetra-GNC antibody, SI-35E20, induces RTCC to NucRed-transduced lung cancer cells A549 and suppresses the growth of lung adenocarcinoma cells in the presence of PBMC;

FIG. 10 shows that a tetra-GNC antibody, SI-38E17, induces RTCC to Nuc-GFP Nalm-6 leukemic cells, in the presence and absence of and donor PBMC;

FIG. 11 shows that a tetra-GNC antibody, SI-39E18, induces RTCC to kill NucRed+UMUC3viii cells derived from human bladder cancer, in the presence of donor PBMC versus a vehicle control;

FIG. 12 shows that a tetra-GNC antibody, SI-38E17, is effective in suppressing the growth of human B cell leukemia cells in a human tumor xenograft model by administrating JVM-3 cells and donor PBMC (5×10⁶ and 2×10⁷, respectively) into NCG mice;

FIG. 13 shows that a tetra-GNC antibody, SI-39E18 is effective in suppressing the growth of human bladder cancer cells in a human tumor xenograft model by administrating UM-UC-3-EGFR VIII cells and human PBMC (5×10⁶ and 5×10⁶, respectively) into NCG mice;

FIG. 14 shows the necessity of simultaneously targeting immunomodulatory proteins, such as PD-L1 immune checkpoint and 4-1BB activation as a hexa-GNC antibody (SI-55H11) mediates more complete elimination of human cervical cancer cells (Hela) than a comparable tri-specific antibody did in a TDCC assay; and

FIG. 15 shows that the improved potency of a hexa-GNC antibody (SI-55H11) due to an additional binding to HER3 as compared to that of its parental penta-GNC antibody (SI-55H9) in TDCC assay using human pancreatic cancer cells (BxPC3) that express low levels of HER3.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present application relates to guidance and navigation control (GNC) proteins and methods of making and using thereof. In some embodiments, the GNC proteins may be multi-specific antibody-like proteins. In some embodiments, the GNC proteins may be multi-specific antibodies, in which cases the GNC proteins may also be referred to as GNC antibodies. In some embodiments, the application provides penta-specific antibody-like proteins and hexa-specific antibody-like proteins. In some embodiments, the application provides penta-specific antibodies and hexa-specific antibodies.

The GNC proteins include the proteins linking multiple functionally independent binding moieties into a single entity that is capable of bringing both effector cells and target cells together (see Applicant's application WO/2019/005642, incorporated herein in its entirety). In one embodiment, these multi-specific binding molecules targeting tumor antigens and immune-activating receptors can utilize similar mechanisms of immune effector cell-mediated killing of tumors at a fraction of the cost. Rather than genetically modifying individual patient T cells, such multi-specific binding molecules can be efficiently manufactured large-scale and administered in a more general off-the-shelf manner. Of the GNC proteins, multi-specific antibodies, such as tetra-specific antibodies, have been shown be able to exert desirable multi-facet GNC effects with structurally and functionally diverse but relatively independent binding domains (see Applicant's application WO/2019/191120, incorporated herein in its entirety).

In one embodiment, the GNC protein may include a multi-specific antibody-like protein comprising a heavy chain and a light chain moiety. The antibody's Fab region is composed of one constant and one variable domain from the heavy and the light chain moiety. The heavy chain may further include three additional antigen-specific binding domains attached to the N-terminal, the C-terminal, or both terminals. The light chain moiety may include one or two additional binding domains attached to the N-terminal, C-terminal, or both terminals.

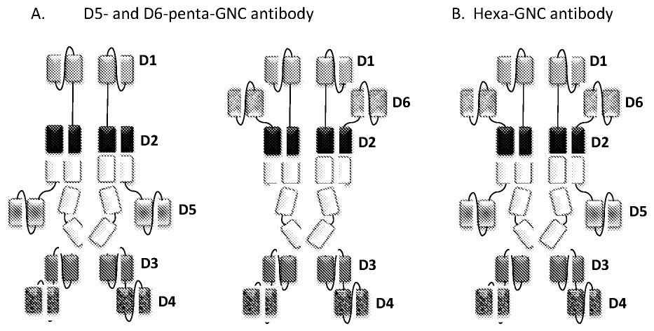

In some embodiments, the GNC antibodies may be penta-GNC antibodies or hexa-GNC antibodies, as shown in FIG. 1. The GNC antibodies may have the ability of directing immune cells (or other effector cells) to tumor cells (or other target cells) through the binding of multiple surface molecules on an immune cell and a tumor cell. The immune cells may be the cells of human immune system, including without limitation, leukocytes, peripheral blood mononuclear cells (PBMC), T cells, and natural killer cells (NK cells). Other target cells may include, without limitations, autoimmune cells (normal B cells), tissue target cells, non-tumor cells, infected cells, inflammatory cells, and damaged cells. In some embodiments, T cells comprises human T cells, including without limitation, naïve T cells, activated T cells, helper T cells, regulatory T cells, memory T cells, and exhausted T cells. In one embodiment, the tumor cells express tumor antigens, including without limitation, tumor-specific antigens (TSA), neoantigens, and tumor-associated antigens (TAA).

In one embodiment, the GNC antibodies may include at least one binding domain capable of binding to one surface molecule on a T cell and at least one binding domain capable of binding to one surface antigen on a tumor cell (Table 1). In some embodiments, the surface molecules on a T cell comprise signaling proteins, including without limitation, CD3, NKG2D, and 4-1BB; the surface molecules on a NK cell comprise signaling proteins, including without limitation, NKG2D and 4-1BB; and the surface antigens on a tumor cell comprise tumor antigens, including without limitation, EGFR, HER2, HER3, MSLN, CD19, and PD-L1. In one embodiment, the tumor cells constitute a tumor or a cancer, including without limitation, a solid tumor, a sarcoma, a hematopoietic malignancy, a lung cancer, a pancreatic cancer, a bladder cancer, a cervical cancer, a breast cancer, a leukemia, and a lymphoma.

The GNC antibodies having at least four additional binding domains in addition to the D2 may require structural stability to maintain independent function of binding specificity and affinity of each binding domain. Each additional binding domain may include a (GₓS_{y})ₙ peptide linker, wherein n is an integer from 1 to 10, x is an integer from 1 to 10, and y is an integer from 1 to 10.

In one embodiment, the binding domain such as D1, D2, D3, D4, D5, or D6 may be a single chain variable fragment (scFv), a receptor, or a ligand (Table 1). A scFv domain may be configured to have a fusion of the variable regions of the heavy (V_{H}) and light chain (V_{L}) in either the V_{H}-V_{L} (HL) or V_{L}-V_{H} (LH) orientation. In one embodiment, the scFv domain may be a stapled structure by introducing a disulfide bond between V_{H}44 and V_{L}100 (Kabat). In one embodiment, the V_{H} region for V_{H3}-containing scFv on any light chain moiety has a R19S mutation (Kabat numbering).

The binding domain may be configured to bind to at least one epitope of an antigen, including without limitation, CD3, 4-1BB, EGFR, HER2, HER3, MSLN, CD19, and PD-L1. The amino acid sequences selected to encode the anti-EGFR binding domain may be humanized sequences. In other embodiments, the amino acid sequences selected to encode the anti-CD19 binding domain are humanized sequences.

In one embodiment, the binding domain may be receptors. In one embodiment, the receptor may be NKG2D. In one embodiment, the D2 may include NKG2D.

The binding domain may be ligands for a receptor such as 4-1BBL (a 4-1BB receptor ligand) and 4-1BBL trimer for 4-1BB, a receptor.

The terms “a”, “an” and “the” as used herein are defined to mean “one or more” and include the plural unless the context is inappropriate.

The term “antibody” is used in the broadest sense and specifically covers single monoclonal antibodies (including agonist and antagonist antibodies), antibody compositions with polyepitopic specificity, as well as antibody fragments, such as Fab, F(ab′)2, and Fv, so long as they exhibit the desired biological activity. In some embodiments, the antibody may be monoclonal, chimeric, single chain, multi-specific, multi-effective, human and humanized antibodies. Examples of active antibody fragments that bind to known antigens include Fab, F(ab′)2, scFv, and Fv fragments, as well as the products of a Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind to an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as V_{H}) and a heavy chain constant domain. Each light chain moiety is comprised of a light chain moiety variable domain (abbreviated as V_{L}) and a light chain moiety constant domain. The V_{H} and V_{L} regions can be further subdivided into domains of hypervariable complementarity determining regions (CDR), and more conserved regions called framework regions (FR). Each variable domain (either V_{H} or V_{L}) is typically composed of three CDRs and four FRs, arranged in the following order: FR1, CDR1, FR2, CDR2, FR3, CDR3, FR4 from amino-terminus to carboxy-terminus. Within the variable regions of the heavy and light chain there are binding regions that interacts with the antigen.

The term of “monoclonal” antibody as used herein include “monoclonal mono-specific”, “chimeric”, and “multi-specific” antibodies (immunoglobulins) in which a portion of the heavy and/or light chain moiety is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., PNAS USA, 1984). Monoclonal antibodies can be produced using various methods, including without limitation, mouse hybridoma, phage display, recombinant DNA, molecular cloning of antibodies directly from primary B cells, and antibody discovery methods (see Siegel. Transfus. Clin. Biol. 2002; Tiller. New Biotechnol. 2011; Seeber et al. PLOS One. 2014).

The term “multi-specific” antibody as used herein denotes an antibody that has at least two binding sites each having a binding affinity to an epitope of an antigen. The term “bi-specific, tri-specific, tetra-specific, penta-specific, or hexa-specific” antibody as used herein denotes an antibody that has two, three, four, five, or six antigen-binding sites. For example, the antibodies disclosed herein with five binding sites are penta-specific, with six binding sites are hexa-specific.

The term “guidance and navigation control (GNC)” protein refers to a multi-specific protein capable of binding to at least one effector cell (such as immune cell) antigen and at least one target cell (such as tumor cell, immune cell, or microbial cell) antigen. The GNC protein may adopt an antibody-core structure including a Fab region and Fc region with various binding domains attached to the antibody-core, in which case the GNC protein is also termed GNC antibody. The GNC protein may adopt an antibody-like structure, in which case the Fv fragment may be replaced with a non-antibody based binding domain such as NKG2D, 4-1BBL (a 4-1BB receptor ligand), 4-1BBL trimer for 4-1BB, or a receptor.

The term “GNC antibody” refers to a GNC protein had an antibody structure that is capable of binding to at least one effector cell (such as immune cell) and at least one target cell (such as tumor cell, immune cell, or microbial cell) simultaneously. The term “bi-GNC, tri-GNC, tetra-GNC, penta-GNC, or hexa-GNC” antibody as used herein denotes a GNC antibody that has two, three, four, five, or six antigen-binding sites, of which at least one antigen-binding site has the binding affinity to an immune cell and at least one antigen-binding site has the binding affinity to a tumor cell. In one embodiment, the GNC antibodies disclosed herein have five to six binding sites (or binding domain) and are penta-GNC and hexa-GNC antibodies, respectively. In some embodiments, the GNC antibodies include antibody binding domains (such as Fab and scFv) without the requirement for additional protein engineering in the Fc region. In one embodiment, the GNC antibody may include a Fc region that is engineered to eliminate effector cell function such as ADCC, ADCP, CDC. Mutations include, but are not limited to L234A/L235A/G237A/K322A and L234A/L235A/K322A (Eu numbering). In one embodiment, mutation of the Fc glycosylation site, e.g, N297A (Eu), may be used to prevent glycosylation and disrupt Fc effector functions. In one embodiment, the GNC antibody as used herein comprises symmetric antibodies that do not require Fc engineering to drive proper assembly of the full protein. In contrast, many existing bi-specific and multi-specific antibody formats require a heterodimerizing Fc in order to combine different specificities into asymmetric molecules. In one embodiment, the GNC antibodies additionally have the advantage of retaining bivalency for each targeted antigen. Further in one embodiment, the GNC antibodies have the advantage of avidity effects that result in higher affinity for antigens and slower dissociation rates. This bivalency for each antigen is in contrast to many multi-specific platforms that are monovalent for each targeted antigen, and thus often lose the beneficial avidity effects that make antibody binding so strong.

The term “humanized antibody” antibody refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art (see Queen et al., Proc. Natl Acad Sci USA, 1989; Hodgson et al., Bio/Technology, 1991). In one embodiment, the “humanized antibody” may be obtained by genetic engineering approach that enables production of affinity-matured humanlike polyclonal antibodies in large animals such as, for example, rabbits (see U.S. Pat. No. 7,129,084).

The term “antigen” refers to an entity or fragment thereof which can induce an immune response in an organism, particularly an animal, more particularly a mammal including a human. The term includes immunogens and regions thereof responsible for antigenicity or antigenic determinants.

The term “epitope”, also known as “antigenic determinant”, is the part of an antigen that is recognized by the immune system, specifically by antibodies, B cells, or T cells, and is the specific piece of the antigen to which an antibody binds.

The term “immunogenic” refers to substances which elicit or enhance the production of antibodies, T-cells, or other reactive immune cells directed against an immunogenic agent and contribute to an immune response in humans or animals. An immune response occurs when an individual produces sufficient antibodies, T-cells, and other reactive immune cells against administered immunogenic compositions of the present application to moderate or alleviate the disorder to be treated.

The term “tumor antigen” as used herein means an antigenic molecule produced in tumor cells. A tumor antigen may trigger an immune response in the host. In one embodiment, the tumor cells express tumor antigens, including without limitation, tumor-specific antigens (TSA), neoantigens, and tumor-associated antigens (TAA).

The term “specific binding to” or “specifically binds to” or “specific for” a particular antigen or an epitope as used herein means the binding that is measurably different from a non-specific interaction. Specific binding can be measured by determining binding of a molecule compared to binding of a control molecule, which generally is a molecule of similar structure that does not have binding activity. Specific binding can be determined by competition with a control molecule that is similar to the target. Specific binding for a particular antigen or an epitope can be exhibited by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹, alternatively at least about 10⁻¹⁰ M, at least about 10⁻¹¹ M, at least about 10⁻¹² M, or greater, where KD refers to a dissociation rate of a particular antibody-antigen interaction. In some embodiments, a multi-specific antibody that specifically binds to an antigen will have a KD that is 20-, 50-, 100-, 500-, 1000-, 5,000-, 10,000- or more times greater for a control molecule relative to the antigen or epitope. Also, specific binding for a particular antigen or an epitope can be exhibited by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The term “stapled” means two domains are covalently linked. In one embodiment, the two domains may be covalently linked through at least one disulfide bond. For example, a scFv domain that has at least one disulfide bond linking VH and VL is called a stapled scFv; and a Fab region that has at least one disulfide bond linking the light chain moiety and the heavy chain is called a stapled Fab.

### EXAMPLES

While the following examples are provided by way of illustration only and not by way of limitation. Those of skill in the art will readily recognize a variety of non-critical parameters that could be changed or modified to yield essentially the same or similar results.

### Example 1. Stapled Binding Domains and Stability of Penta- and Hexa-GNC Antibody

In multi-specific GNC antibodies such as tetra-GNC antibodies, the heavy chain may comprise up to three scFv domains plus the Fab region to constitute four binding specificities, whereas the light chain remains unmodified. In penta-GNC antibodies, one scFv domain is added to either N-terminus or C-terminus of the light chain to gain the fifth binding specificity, as shown in FIG. 1 and Table 1. When an scFv domain is attached to each of the N- and C-terminus of the light chain, the antibody gains the fifth and sixth binding specificity and is classified as a hexa-GNC antibody. The modifications to both heavy chain and light chain posed uncertainty to the stability of the antibody. To maintain the stability and independence of all individual binding domains in either a penta- or a hexa-GNC antibody, one option is to introduce, i.e. staple, a disulfide bond at V_{L}100 and V_{H}44 (Kabat) to each Fv fragment and scFv domain. A disulfide bond between V_{L} and V_{H} may be used for all scFv domains to stabilize the overall structure. Alternatively, a disulphide bond may be introduced into at least one selected scFv domain at any position.

A pair of penta-GNC antibodies (SI-1P1 and SI-1P2) (SEQ ID NO. 1-4 and 5-8, respectively) with identical binding specificities were created for analysing the effect of stapled scFv domains. The heavy chain of the two antibodies comprises αCD3 scFv at D1, αEGFR V_{H} at D2 (in the Fv-CH1-Fc configuration), αPD-L1 at D3, and α4-1BB at D4, and the light chain comprises αEGFR V_{L} and αHER3 scFv at D5 according to the naming system in FIG. 1. SI-1P2, but not SI-1P1, comprises “stapled” scFv domains at D1, D3, D4, and D4, namely, αCD3 scFv[V_{H}44 G->C V_{L}100 G->C] at D1, αPD-L1 scFv[V_{H}44 G->C V_{L}100 G->C] at D3, α4-1BB scFv[V_{H}44 G->C V_{L}100 G->C] at D4 on its heavy chain, and αHER3 scFv[V_{H}44 G->C V_{L}100 G->C] on its light chain (Kabat numbering) as listed Table 1.

Both SI-1P1 and SI-1P2 were cloned into vector pTT5 following a modular cloning strategy using restriction sites HindIII/SalI/NheI/BamHI/BspEI/PacI. These penta-GNC antibody constructs were expressed with acceptable titers using both HEK and ExpiCHO expression systems for 5 and 9 days, respectively, and purified with 5 mL MabSelect protein A columns followed by Size Exclusion using a hiload 16/600 200 pg preparative SEC column on either an Akta Avant or Purifier system. SEC aggregates were analyzed using a waters HPLC linked to multi angle light scattering (MALS, Wyatt systems) to identify correct molecular weight by do/dc calculated methods. Dynamic light scattering (Wyatt systems) was used in the further analysis to determine the melting temperature of the produced protein. With all of the analyses conducted as shown in Table 2, the disulfide bonded, i.e. “stapled”, penta-GNC antibodies displayed more stable characteristics.

Antibody-based proteins are most often purified via protein A affinity chromatography, where the protein A resin captures the antibody at a binding site at the C_{H2}-C_{H3} interface in the Fc domain. However, protein A also binds to the V_{H} domain of V_{H3} family Fvs. For most antibody-based platforms this is not a problem, since V_{H} domains are generally on the heavy chain. However, when scFvs containing V_{H3} are attached to the light chain, the V_{H} domain can bind to protein A resin during purification, causing light chain monomers and dimers to contaminate the desired heavy-light chain heterotetramer. Thus, a potential hurdle when producing multi-specific antibodies containing any V_{H3} domain on the light chain is the presence of additional contaminants in the protein A elution. This is especially problematic when the light chain expresses more efficiently than the heavy chain, causing an abundance of light chain contaminants to be purified along with the desired protein assembly.

In order to rationally disrupt protein A binding of V_{H3} family members, a structural approach was taken to interrupt the binding interface. Crystal structure 1DEE (Graille M. et al. Proc. Nat. Acad. Sci. 2000.) showed that residue R19 in V_{H3} (Kabat numbering) is in direct contact with two side chains of protein A domain D. In particular, contact with Q32 and D36 could be eliminated to significantly weaken the interaction. Thus, R19 was mutated to serine, which does not form these interactions due to its shorter side-chain. Additionally, S19 exists naturally in other VH family members, suggesting that it may be less immunogenic than other substitutions.

The mutation R19S (Kabat numbering) was incorporated into the FR1 region of the VH domain for V_{H3}-containing scFvs on the GNC light chain. Specifically, the penta-GNC antibody, SI-77P1 (SEQ ID NO. 41-44), harbored R19S mutation in its light chain sequence encoding the anti-CD19 scFv at domain 6, and the hexa-GNC antibodies, SI-55-H11 (SEQ ID NO. 53-56), SI-55H12 (SEQ ID NO. 57-60), SI-77H4 (SEQ ID NO. 61-64), and SI-77H5 (SEQ ID NO. 65-68) harbored R19S mutation in their light chain sequences encoding the anti-HER3 scFv domain at D5 and the anti-CD19 scFv at domain 6. The residue of interest is at the protein A binding interface (4), and therefore mutation of R to S disrupts the interaction with protein A. Elimination of protein A binding in light chain scFvs prevents light chain monomers and dimers from binding to protein A during purification. As a result, a more homogeneous product without light chain contaminants can be obtained. For hexa-GNC, which may contain up to two V_{H3} scFvs per light chain, this mutation is especially important in allowing efficient purification of the desired product.

Wild-type IgG1 antibodies contain an active Fc domain which binds to Fc gamma receptors on immune cells, as well as C1q, the first component of the complement cascade. These binding capabilities allow antibodies with active Fc to elicit effector functions including antibody-dependent cellular cytotoxicity (ADCC), antibody-dependent cellular phagocytosis (ADCP), and complement-dependent dependent cytotoxicity toward antigen-bearing cells. However, in the context of T cell redirection, an active Fc domain can exacerbate cytokine release syndrome and cause off-target cytotoxicity (Strohl & Naso, Antibodies, 2019). Therefore, null Fc domains incorporating silencing mutations that weaken binding to Fc gamma receptors and complement can decrease cytokine release syndrome, and even increase efficacy of T cell redirecting antibodies by increasing infiltration into the tumor (Wang et al., Cancer Immunol. Res. 2019). Many point mutations have been introduced to weaken interaction with Fc gamma receptors or C1q, and to lessen Fc effector functions (Saunders, Frontiers Immunol. 2019). Among these, the L234A, L235A, and G237A mutations have been shown to decrease ADCC and ADCP through decreased binding to Fc gamma receptors. The mutation K322A has been shown to decrease binding to C1q, and therefore ablate CDC. Furthermore, mutation of N297A removes the Fc glycosylation site, generating aglycosylated Fc domain that does not interact as strongly with its receptors.

Fc silencing mutations were incorporated into the GNC platform in order to generate therapeutics with mitigated risk of cytokine storm and improved tumor penetration qualities due to less binding of Fc gamma receptors in the periphery. Example molecules contained different null Fc versions to demonstrate that an array of Fc archetypes could be used in the GNC platform. Thus, mutations used to modulate effector function of monoclonal and multi-specific antibodies can also be efficiently incorporated into the GNC platform.

### Example 2. Optimizing Binding Domains

While selecting binding specificities dictates the utility of a multi-specific GNC antibody, optimizing commonly used binding domains may improve the efficacy of the antibody. The penta-GNC and hexa-GNC antibodies (collectively known as GNC antibodies as listed in Table 1) were cloned, expressed, and produced following the similar materials and methods as described for producing SI-1P1 and SI-1P2 antibodies in Example 1.

The anti-CD3 variable domain sequences, 284A10 (Applicant's application No. PCT/US2018/039143, incorporated herein in its entirety), were used as unmodified, stapled (284A10 stapled, SEQ ID NO. 89-92), humanized (284A10 H1, SEQ ID NO. 93-96), or humanized and stapled (284A10 H1 stapled, SEQ ID NO. 97-100) sequences to encode either a scFv (in either V_{H}-V_{L} or V_{L}-V_{H} orientation) or a Fab of the heavy chain monomer. The other anti-CD3 variable domain sequences, 283E3, were identified, cloned, and humanized as 283E3 H1 (SEQ ID NO. 101-104), which were used to encode the Fab region at D2 of the heavy chain monomer for each of seven penta-GNC antibodies as indicated in Table 1.

The anti-PD-L1 variable domain sequences, PL221G5 (Applicant's application No. PCT/US2018/039144, incorporated herein in its entirety), were humanized and used to encode either a “unstapled” or a “stapled” (SEQ ID NO. 105-108) scFv domain at D3 of the heavy chain monomer.

The anti-4-1BB variable domain sequences, 466F6 (Applicant's application No. PCT/US2018/039155 incorporated herein in its entirety), were humanized and used to encode either a “unstapled” or a “stapled” (SEQ ID NO. 109-112) scFv domain at D4 of the heavy chain monomer.

### Example 3. Octet Analysis of Binding Affinity

To assess the functionality of the GNC antibodies, the binding affinity of each individual domain of penta-GNC antibodies was carried out by Biolayer Interferometry (Octet 384 system). The penta-GNC antibodies were captures onto the probe using anti-human Fc (AHC tips), and individual epitopes (CD3ε/δ and EGFR were produced, 4-1BB and PD-L1 (Acro Biosystems) were used as analyte to determine the disassociation constant (K_{D}) by kinetic methods (K_{off}/Kₒₙ). As shown in Table 3, all of the binding constants of the individual domains in either SI-1P1 or SI-1P2 were within the reported ranges and the previously determined individual affinities of either monoclonal antibodies or scFv molecules alone.

Octet analysis was used to ensure that GNC antibodies retain their binding to all of their cognate antigens. GNC antibodies were loaded onto AHC sensors for 180 seconds at 10 ug/ml, followed by a 60-second baseline step, a 180-second association step with 100 nM of commercially purchased human antigen, and a 360-second dissociation step. Samples for all steps were in Octet buffer (PBS containing 0.1% Tween 20 and 1% BSA). Fits were performed using a 1:1 binding model to extract affinity KD values, reported in Table 4. The data implies that each binding domain retains its binding affinity when placed at different positions of the GNC antibodies.

### Example 4. T-Cell Activation

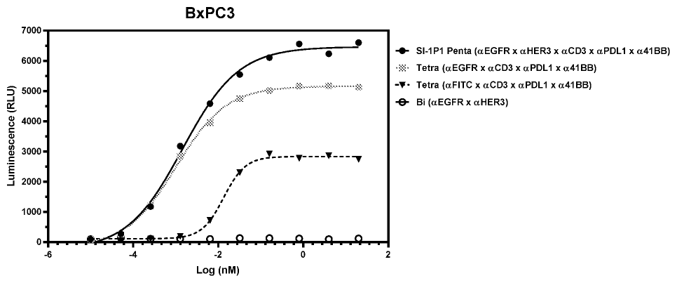

To validate the functionality of the GNC antibodies, penta-GNC antibodies were assessed for T cell activation. The T cell activation assay was performed to compare the potency of SI-1P1, which binds to both EGFR and HER3, with that of an EGFR-tetra-GNC antibody (which binds to EGFR but not HER3), a FITC-tetra-specific antibody (which does not bind to either EGFR or HER3), and a bispecific antibody (which only binds to EGFR and HER3). Human pancreatic cancer cells (BxPC3) were used as target cells, which express high levels of EGFR and low levels of HER3 (Table 5). BxPC3 cells were plated in quadruplicate using a BioTek EL406 in 384 well plates at a density of 1500 cells/well after lifting with disassociation reagent (TrypLE Express) and allowed to adhere for 24 hours. Following this, Jurkat CD3 NFAT Effector cells were added at a cellular ratio of 5:1 (Jurkat Lucia Cells, Invivogen) and the GNC antibody was added in a 10-point 10-fold serial dilution from 50 nM to 0.5 fM and incubated for 4 Hours. Readout was performed by the addition of Promega Bright-Glo reagent and luminescence was measured on a Clariostar Plus microplate reader (BMG-Labtech). Data was plotted in log scale with Graphpad prism and fit to a nonlinear variable slope equation (FIG. 2). The data indicates that the penta-GNC antibody (SI-1P1) exerts similar potency (SI-1P1=1.456 pM, EGFR-tetra-GNC=1.028 pM, FITC-tetra-GNC=13.41 pM) and higher maximum T-cell activation (SI-1P1=6464, EGFR-tetra-GNC=5161, FITC-tetra-GNC=2835) as compared to control antibodies (EGFR-tetra-GNC antibody, FITC-tetra-GNC antibody, and a control bispecific antibody (Table 6).

### Example 5. T-Cell Dependent Cellular Cytotoxicity (TDCC)

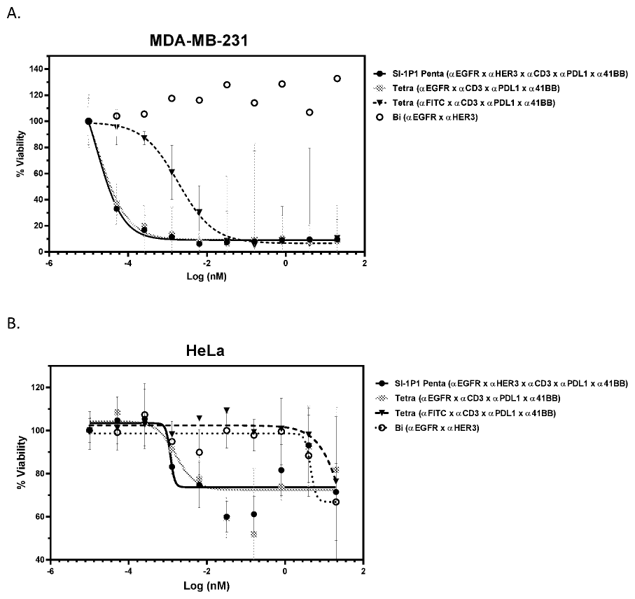

TDCC is a standard feature of antibody therapy for treating cancer other diseases. To assess the TDCC mediated by the GNC antibodies, SI-1P1 (a penta-GNC antibody capable of binding to tumor antigens EGFR and HER3) was used to compare with control antibodies, including a EGFR-tetra-GNC antibody that only binds to EGFR, a FITC-tetra-specific antibody that does not bind either EGFR or HER3, and a control bispecific antibody that only binds to tumor antigens EGFR and HER3 (Table 6). Serial dilutions (0 to 30 nM; 1 to 5 dilution factor) of antibodies were added to a white 384-well plate containing luciferized MDA-MB-231 or HeLa cells (both have high EGFR and low HER3, see Table 5 and plated 24 hours prior and grown at 37° C.) and activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. Data were fit to a sigmoidal function to calculate EC50 values (FIG. 3). For MDA-MB-231 cells as shown in FIG. 3A, the EC50 was 0.01575 pM (SI-1P1), 0.01646 pM (an EGFR-tetra-GNC control antibody), and 1.882 pM (FITC-tetra-specific control antibody), and for HeLa cells as shown in FIG. 3B, the EC50 was 1.161 pM (SI-1P1), 1.635 pM (EGFR-tetra-GNC control antibody), 3736200 pM (FITC-tetra-specific antibody), and 4500 pM (bispecific control antibody). The data demonstrate that, with an increased number of binding specificities, the penta-GNC antibody exerts higher TDCC potency as compared to control antibodies with fewer binding specificities.

### Example 6. NKG2D Receptor as a Binding Domain

An increase number of binding specificities allows the GNC antibodies to bind not only T cells but also subsets of T cells, natural killer cells, and other types of immune cells. On the hand, an added binding specificity may replace the cellular response to or recognition of targeted cells. For example, NKG2D is a major recognition receptor for the detection and elimination of transformed and infected cells as its ligands are induced during cellular stress, either as a result of viral infection or genomic stress such as in cancer. In humans, NKG2D is expressed by NK cells, γδ T cells, and CD8+ αβ T cells. In NK cells, NKG2D serves as an activating receptor, which itself is able to trigger cytotoxicity, whereas on CD8⁺ T cells the function of NKG2D is to send co-stimulatory signals to activate them. The addition of NKG2D as a binding specificity for the GNC antibodies may improve the cytotoxicity and efficacy of the antibody as a single multi-functional therapeutic agent. In this context, penta-GNC antibodies, SI-49P1 and SI-49P3 (SEQ ID NO. 17-20 and SEQ ID NO. 21-24, respectively), were created by adding NKG2D receptor as a binding domain at D5 (Table 1). The affinity of NKG2D of SI-49P3 (Table 4) for human MICA was founded to be within the expected range, indicating that NKG2D can act as a receptor for the penta-GNC antibody to bind its ligand.

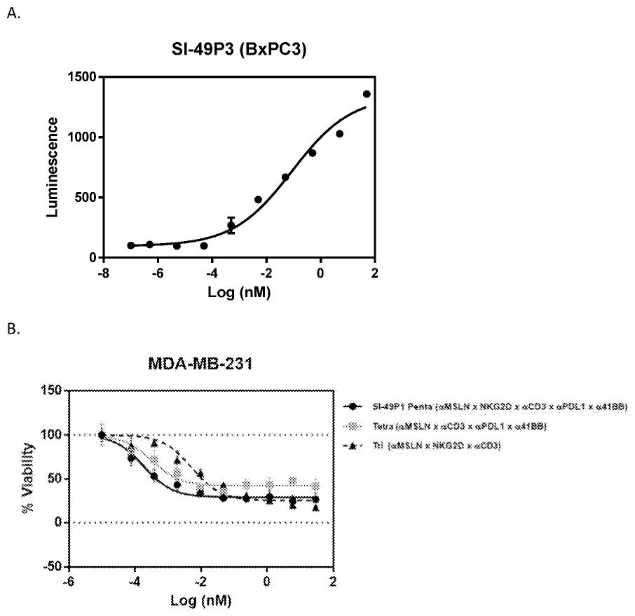

Both SI-49P3 and SI-49P1 are capable of recognizing one tumor antigen via the Fab region while extending multiple binding specificities to CD3, PD-L1, 4-1BB, and NKG2D. To demonstrate that SI-49P3 retains its ability in T cell activation, BxPC3 target cells were plated in quadruplicate using a BioTek EL406 in 384 well plates at a density of 1500 cells/well after lifting with disassociation reagent (TrypLE Express) and allowed to adhere for 24 hours. Following this, Jurkat CD3 NFAT Effector cells were added at a cellular ratio of 5:1 (Jurkat Lucia Cells, Invivogen) and GNC reagent was added in a 10-point 10-fold serial dilution from 50 nM to 0.5 fM and incubated for 4 Hours. Readout was performed by the addition of Promega Bright-Glo reagent and luminescence was measured on a Clariostar Plus microplate reader (BMG-Labtech). Data was plotted in log scale with Graphpad prism and fit to a nonlinear variable slope equation as shown in FIG. 4A. The data demonstrate that the addition of NKG2D receptor does not affect T cell activation and the penta-GNC antibody is capable of eliciting potent T cell activation (EC50=88.1 pM) while simultaneous engaging T cell antigens and targeting tumor cells.

To assess TDCC of the NKG2D class of penta-GNC antibodies, SI-49P1 was used. The control antibodies included a tri-GNC antibody lacking the binding specificities to both PD-L1 and 4-1BB, and a tetra-GNC antibody lacking NKG2D receptor. Serial dilutions (0 to 30 nM; 1 to 5 dilution factor) of GNC protein were added to a white 384-well plate containing luciferized MDA-MB-231 cells (MICA and mesothelin expression; plated 24 hours prior and grown at 37° C.) and activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. Data were fit to a sigmoidal function to calculate EC50 values of 0.1865 pM (SI-49P1), 0.3433 pM (tri-GNC control antibody), and 5.356 pM (tetra-GNC control antibody) (FIG. 4B). The data indicates that the addition of NKG2D receptor to the tetra-GNC antibody improves the potency of TDCC, and that the addition of both αPD-L1 and α4-1BB domains to a tri-GNC antibody with its binding specificity to NKG2D significantly improves the potency of TDCC. In other words, the GNC antibodies can accommodate multiple binding specificities to modulate, cooperate, and direct an optimized immune response to targeted cells, such as cancer.

### Example 7: NKG2D Receptor at D2 of the Antibody-Like GNC Protein

To test the utility of antibody-like GNC proteins with natural receptors at position D2, the sequence for human NKG2D (residues F78 through V216) was cloned in place of V_{H} and V_{L} domains at position D2 in the context of expression plasmids encoding SI-49P10 (αCD3×NKG2D×αPD-L1×α4-1BB×αEGFR, SEQ ID NO. 117-200). SI-49P10 was expressed following the materials and methods above, and had exceptionally low aggregation (95.64% peak of interest by analytical SEC) after protein A purification, indicating that the antibody-like GNC proteins containing a non-antibody binding moiety, such as NKG2D receptor, in the D2 position of the heavy chain have the potential to be highly stable. Penta GNC proteins SI-49P6 (αCD3×NKG2D×αPD-L1×α4-1BB×αCD19, SEQ ID 123-126) and SI-49P7 (αCD3×NKG2D×αPD-L1×41BBL trimer×αCD19, SEQ ID 127-130) were similarly cloned, expressed, and purified.

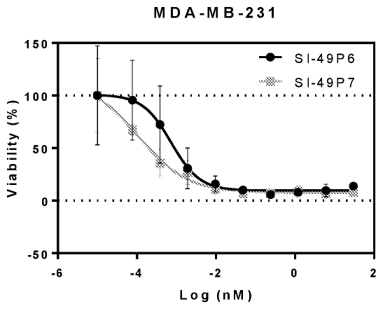

To ensure that the NKG2D dimer retained full functionality, Octet binding to human MICA was assessed. SI-49P10 was loaded onto AHC tips and bound to His-tagged MICA. The extracted KD values confirmed that NKG2D retains binding activity when present in the D2 position (Table 4). SI-49P10 had a K_{D} value of 1.84 nM. As a comparison, SI-49P3 (NKG2D dimer in D5) had a similar K_{D} value of 1.39 nM. The other domains of SI-49P10 also retained high binding affinity to their cognate antigens (Table 4). Similarly, binding of SI-49P6 and SI-49P7 for MICA was determined by loading biotinylated human MICA onto SA tips and observing binding to serial dilution of GNC proteins (0 to 100 nM) as analytes. The KD resulting K_{D} values were 7.763 nM (SI-49P6) and 10.67 nM (SI-49P7), again confirming the retention of target binding by receptor proteins in the D2 position (Table 4). These K_{D} values with antigen as the loaded ligand were slightly lower affinity compared to the experiment in which GNC protein was loaded, possibly due to inactive conformation or incompletely exposed epitope of the MICA protein when it is loaded as ligand. Nevertheless, the potent femtomolar (<1 pM) TDCC elicited by these proteins with NKG2D in D2 position toward MICA-bearing MDA-MB-231 cells (FIG. 8), suggests that the NKG2D receptor retains active binding in the D2 position. Thus, the GNC platform is highly adaptable with regard to where each domain is placed.

### Example 8. 4-1BB Ligand as a Binding Domain

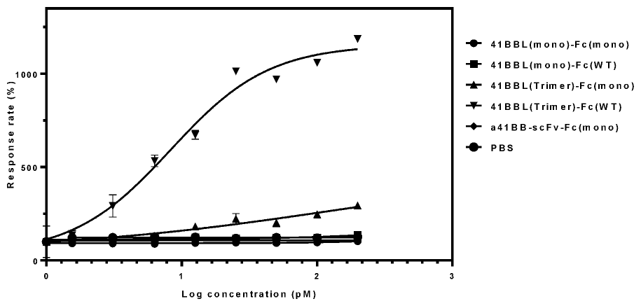

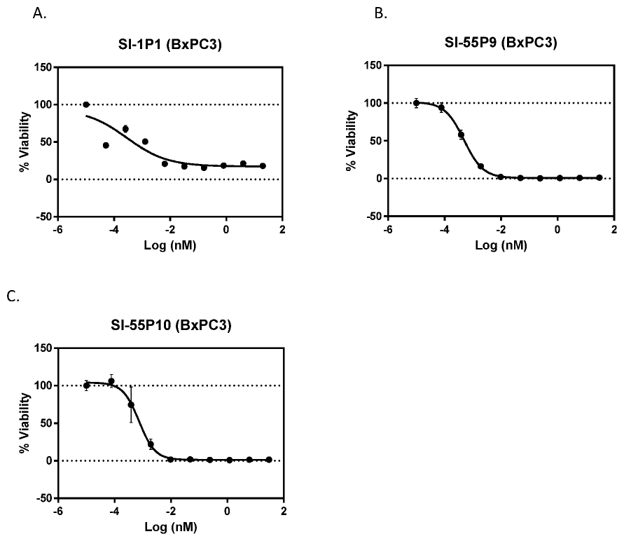

4-1BB is a co-stimulatory immune checkpoint TNFR receptor expressed by activated T cells and NK cells. Its activation by 4-1BB ligand or by an agonist antibody on CD8+ T cells results in increased proliferation, cytokine production, and survival. To optimize the 4-1BB mediated immune response, 4-1BB activation reporter bioassay was performed to assess the functionality of different domains. The 4-1BB activation assay is based on the methods followed by Promega 4-1BB Bioassay kit (SKU: JA2351). The assay consists of a genetically engineered Jurkat T cell line that expresses human 4-1BB and a luciferase reporter driven by a response element that can respond to 4-1BB ligand/agonist antibody stimulation, called 4-1BB Effector Cells. 4-1BB effector cells are cultured in RPMI-1640 with 10% FBS. Before the assay, the cells are counted and re-plated into 384 well (Corning 3570) at 500 cell/well. Test article experiments are conducted in quadruplicate as the 96 well dilution block is stamped into 384 well quadrants robotically (Opentrons OT-2 liquid handling robot). The 4-1BB assay plate was incubated for 6 hours. Readout of the 4-1BB activation curve was accomplished by the use of the Promega Bright-Glo luciferase assay kit. Briefly, 20 uL were added to the 4-1BB assay plate and incubated for ^{˜}15 min before measuring the resultant luminescence on a BMG Clariostar plate reader. Activation curves were analyzed and plotted in GraphPad Software by 4PL curve (FIG. 5). The results show that the 4-1BB ligand trimer (4-1BBL trimer, SEQ ID NO. 113-114) elicits robust activation of 4-1BB signaling when compared to monomeric 4-1BB ligand, monomeric Fc, and an anti-4-1BB scFv. The penta-GNC antibodies, SI-55P4, SI-55P10, and SI-79P3 (SEQ ID NO. 29-32; 37-40; 49-52, respectively) were created to have 4-1BBL trimer as a binding domain all at D4 (see Table 1, and FIG. 7 below).

Biological activity of the GNC proteins with NKG2D in D2 position was determined using TDCC assay with MICA-bearing MDA-MB-231 target cells (FIG. 8). The ratio of target to effector cells was 1:5 and the assay was conducted for 72 hours after adding drug dilutions and T cells to the tumor cells. The resulting EC50 values were quite potent (SI-49P6, 0.7366 pM and SI-49P7, 0.1094), confirming the ability of NKG2D to target T cells to kill tumor cells. Thus, placing the receptor NKG2D as binding domains in the GNC D2 position results in stable GNC proteins that elicit potent TDCC.

### Example 9. Humanized EGFR Binding Domain

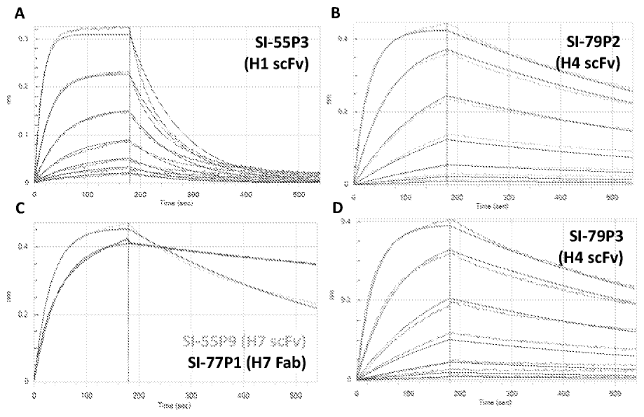

Cetuximab is a chimeric mouse/human monoclonal antibody for treating EGFR-expressing metastatic colorectal cancer, non-small cell lung cancer, and head and neck cancer. Humanized antibody is obtained. In this example, humanized sequences encoding anti-EGFR binding (H1, H4, H7, and H7-stapled) (SEQ ID NO. 69-72; 73-76; 77-80, and 81-84, respectively), were cloned into an expression cassette for producing anti-EGFR (D2) penta-GNC antibody (51-77P1) and anti-EGFR (D1) penta-GNC antibodies (SI-55P3, SI-55P4, SI-79P2, SI-79P3, and SI-55P9)(SEQ ID NO. 25-28; 29-32; 45-48; 49-52; 33-36, respectively) as listed in Table 7. Each expression cassette was transfected into 25 mL of ExpiCHO and expressed for 8 days followed by protein-A affinity chromatography for harvesting and purifying each penta-GNC antibody. The antibodies were produced with good titer (Table 7). Analytical SEC data after protein-A purification demonstrates that the penta-GNC antibody containing a humanized anti-EGFR domain can be expressed with low aggregation (Table 7). Octet was used to verify that the penta-GNC antibodies containing humanized anti-EGFR domains, H1, H4, or H7, can bind to human EGFR, respectively (FIG. 6 and Table 7). Each penta-GNC antibody was loaded via AHC sensors at 10 μg/ml and bound to a serial dilution (highest 200 nM, 1:2.5 dilutions) or a single 100-nM concentration of His-tagged human EGFR. The resulting global fit to a 1:1 binding model demonstrated that the penta-GNC antibodies bind to EGFR with affinities in the low nanomolar range (Table 7).

To produce hexa-GNC antibodies, the humanized anti-EGFR domain, H7 (SEQ ID NO. 77-80), was cloned into an expression cassette for producing anti-EGFR hexa-GNC antibodies. The humanized binding domain was placed at either Fab or as scFv at D1 in hexa-GNC antibodies, SI-77H4 (SEQ ID NO. 61-64) and SI-55H11 (SEQ ID NO. 53-56), respectively. The control antibody, SI-77H5 (SEQ ID NO. 65-68), comprises the anti-EGFR binding Fab region encoded by the Cetuximab mouse sequences. The expression cassette was transfected into 25 mL of ExpiCHO and expressed for 8 days followed by protein A affinity chromatography for harvesting and purifying each hexa-GNC antibody. The hexa-GNC antibodies were produced with good titer (Table 8). Analytical SEC data after protein A purification demonstrates that each hexa-GNC antibody containing a humanized anti-EGFR domain can be expressed with low aggregation (Table 8). Octet was used to verify that each of these hexa-GNC antibodies containing a humanized anti-EGFR domain can bind to human EGFR (Table 4 and 8). The hexa-GNC antibodies were loaded via AHC sensors at 10 μg/ml and bound to a serial dilution (highest 200 nM, 1:2.5 dilutions) or a single 100-nM concentration of His-tagged human EGFR. The resulting global fit to a 1:1 binding model demonstrated that the affinity of each hexa-GNC antibody binding to EGFR was in the low nanomolar range (Table 8).

### Example 10. Humanized CD19 Binding Domain

CD19 is a biomarker for B lymphocyte development and lymphoma diagnosis. CD19-targeted therapies based on T cells that express CD19-specific chimeric antigen receptors (CAR-T) have been utilized for their antitumor abilities in patients with CD19⁺ lymphoma and leukemia, such as non-Hodgkin's lymphoma, chronic lymphocytic leukemia, and acute lymphocytic leukemia. In this context, a humanized CD19 binding domain is desirable.

All computational steps were performed in the Discovery Studio package (Dassault Systemes). First a structural model was generated using the mouse BU12 sequence. Antibody framework regions in the input sequence were identified and aligned to a database of antibody variable domains using Hidden Markov Models (HMM), and this alignment was used to build and score models using the MODELLER software. CDR loop modelling was performed by a structural mapping of the CDRL1 CDRL2 CDRL3 CDRH1 and CDRH2 regions to known canonical classes and loop models were built similarly to the framework. The framework regions from the mouse BU12 antibody were aligned and matched to the closest human germline sequence and CDRs regions were copied into the human sequence with the exception of important structural residues (Vernier residues [Almagro and Fransson, 2008]). Mutations predicted to stabilize the previously build structural model were evaluated computationally by 1000 steps of Steepest Descent with a RMS gradient tolerance of 3, followed by Conjugate Gradient minimization and stabilizing mutations matching frequent human residues were chosen on the basis of individual and combined −ΔΔG versus the initial model. The resulting final sequences were tested for humanness using the Abysis webserver based on the method of Abhinandan and Martin (2007).

To equip penta- or hexa-GNC antibodies with the binding specificity to CD19 for targeting B cell malignancies, the sequences encoding anti-CD19 V_{L} and V_{H} domains were selected from SEQ ID NO. 87, 88, 121, 122, 131, 132 carrying modified V_{L}; and SEQ ID NO. 85, 86, 87, 88 also carrying modified V_{L} along with V_{H} containing R19S mutation (Table 1, also see Example 1 for R19S mutation) and connected with a (G₄S)x4 linker to form the anti-CD19 scFv domain. The corresponding gene sequence was cloned into different positions of penta- or hexa-GNC antibodies using restriction digest into the pTT5 expression plasmid for the appropriate heavy or light chain moiety. The anti-CD19 penta-GNC and hexa-GNA antibodies as listed in Table 1 were produced and characterized as described above. Octet analysis of CD19 binding affinity indicated that each GNC antibody retains CD19 binding affinity in an expected range. when placed on the light chain moiety monomer of the GNC antibodies (Table 4).

### Example 11. Penta-GNC Antibodies with Optimized Binding Domains

With the optimized specific binding for EGFR, CD19, and 4-1BB receptor, the penta-GNC antibodies were assessed in TDCC assay. SI-55P9 and SI-55P10 (SEQ ID NO. 33-36 and 37-40, respectively) are a pair of penta-GNC antibodies with identical binding specificities, except SI-55P9 has a humanized anti-EGFR binding domain and SI-55P10 uses 4-1BBL trimer, as to anti-4-1BB binding domain in SI-55P9, to activate 4-1BB signaling. To assess the effect of these differences in TDCC assay, serial dilutions (0 to 30 nM; 1 to 5 dilution factor) of Penta GNC protein SI-1P1, SI-55P9, and SI-55P10 were added to a white 384-well plate containing luciferized BxPC3 (high EGFR expression) cells (plated 24 hours prior and grown at 37° C.) and activated T cells (plated immediately before drug; effector:target=5:1 for SI-1P1 and 7:1 for SI-55P9 and SI-55P10) in a total volume of 50 ul. After an additional 72 hours, 20 ul of Bright-Glo (Promega) was added to wells, and luminescence corresponding to viability of luciferized tumor cells was determined using a CLARIOstar plate reader. As shown in FIG. 7, the potency of SI-55P9 and SI-55P10 were similar to each other, when compared to that of SI-1P1. Data were fit to a sigmoidal function to calculate EC50 values of 0.2814 pM (SI-1P1), 0.4871 pM (SI-55P9), and 0.7358 pM (SI-55P10), all in the picomolar ranger. The finding implies that the composition of binding specificities dictates the potency of penta-GNC antibodies in the TDCC assay and that other in vitro and in vivo analyses may be used to better evaluate the optimization of each binding domain.

### Example 12. Redirected T Cell Cytotoxicity

Any antibody-based binding domain may be converted to Fab or scFv format and plugged directly into a GNC antibody. For example, the GNC antibodies are characterized by adding the fifth and/or sixth binding domains to the light chain moiety. If the binding specificities on the heavy chain can be dedicated to frequently used targets, such as CD3, PD-L1, and 4-1BB, the utilities of GNC platform may become flexible in terms of selecting targeted tumor antigens and paring the less flexible heavy chain with a desirable light chain moiety. In this context, three tetra-GNC antibodies were selected (from Applicant's application No. PCT/US2019/024105, incorporated herein in its entirety) and evaluated using the in vitro redirected T cell cytotoxicity (RTCC) assay and in vivo human tumor xenograft models.

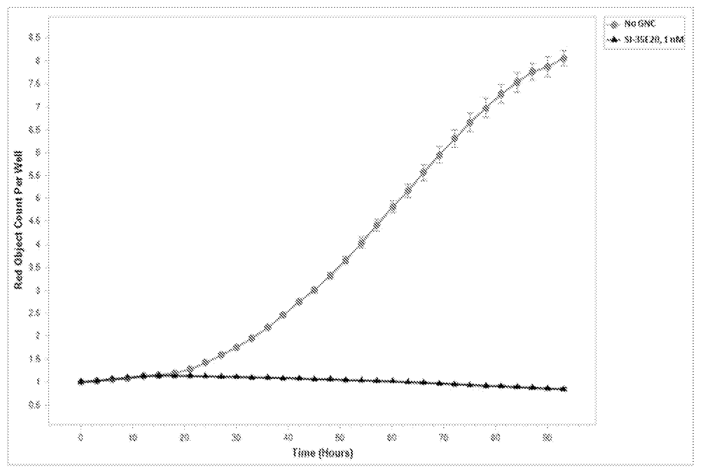

SI-35E20 is a tetra-GNC antibody capable of binding to 4-1BB (D1), PD-L1 (D2), ROR1 (D3), and CD3 (D4) (Table 1). The ability of SI-35E20 to induce RTCC was determined using live cell imaging of cultures containing PBMC (single donor) and red fluorescence-labeled tumor cells over a 4-day period. PBMC (50,000 cells/mL) were used against NucRed-transduced A549 lung adenocarcinoma cells at a ratio of 4:1 for PBMC and A549. The assay wells were set up in triplicate with 1 nM of SI-35E20 or no GNC (buffer alone) as negative control, and proliferation of target cells was monitored over time for 94 hours. The data shows that SI-35E20 is capable of suppressing the growth of targeted cancer cells over time (FIG. 9).

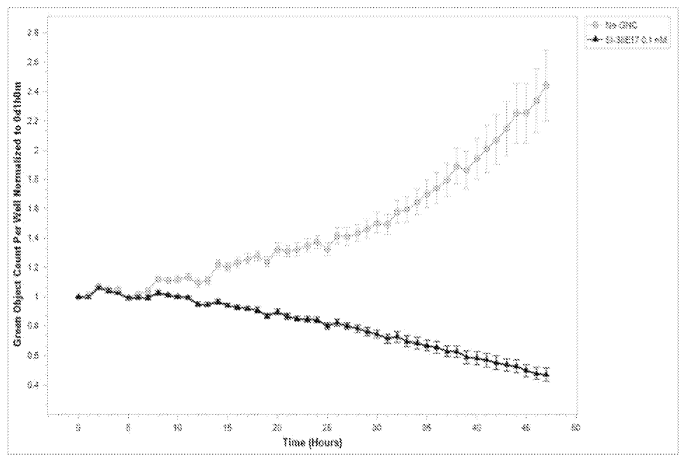

SI-38E17 is a tetra-GNC antibody capable of binding to CD3 (D1), CD19 (D2), PD-L1 (D3), and 4-1BB (D4) (Table 1). To evaluate the effect of SI-38E17-mediated RTCC on cancer cells, Nalm-6 Nuc-GFP (a human leukemic cell line) was used as target cells and PBMC from one donor was used as effector cells. RTCC assay was conducted at E:T ratio=1. At 100 pM, the SI-38E17-mediated RTCC was traced by IncuCyte to detect the proliferation of target cells for 48 hours. SI-38E17 mediated strong RTCC functional activity against Nalm-6 (FIG. 10). The data supports the notion that the tetra-GNC antibody, such as SI-38E17, is capable of suppressing the growth of targeted cancer cells over time.

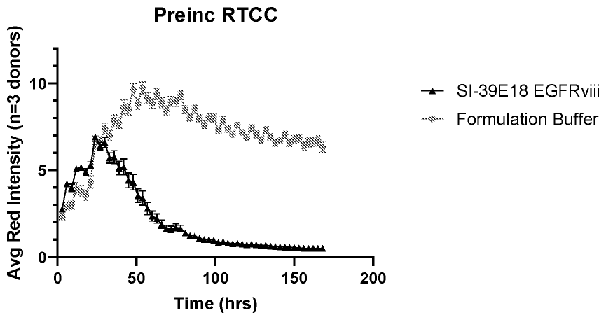

SI-39E18 is a tetra-GNC antibody capable of binding to CD3, EGFRvIII, PD-L1, and 4-1BB. The RTCC assay confirms that SI-39E18 elicits more cell killing than vehicle control as shown in FIG. 11, where the measurement of red fluorescence intensity over time averaged for the three different PBMC donors. In the presence of both SI-39E18 or buffer control, target cells increased in number as measured by fluorescence intensity for the first 24 hours of culture, where the effector cells were preincubated for 3 days with SI-39E18 or control prior to target cell addition. Between 24-48 hours after addition of targets to the culture, the number of target cells began to decline in the presence of SI-39E18 stimulation, but not in the wells containing the buffer control. The more modest loss of target cells in the buffer control sample in both conditions after 48 hours was most likely attributable to depletion of nutrients or an allogeneic T cell response toward the MHC mismatched target cells. The data supports the notion that the tetra-GNC antibody, such as SI-39E18, can prevent the growth of targeted cells over time.

### Example 13. Human Tumor Xenograft Models

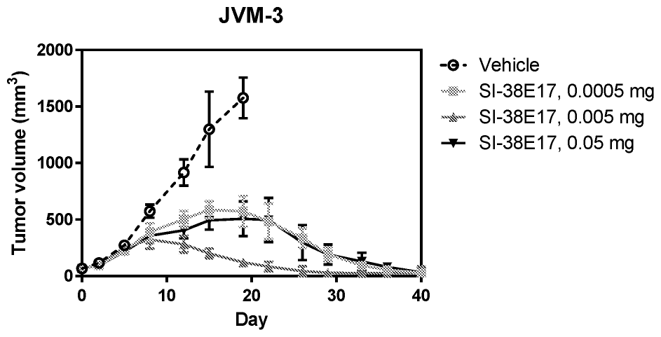

SI-38E17 was tested in a mouse xenograft model to examine its ability to slow tumor growth in vivo (FIG. 12). Human B-cell leukemia cells (JVM-3) were subcutaneously transplanted on the right flank of NCG mice at 5×10⁶ per mice, and donor PBMC was injected intraperitoneally at 2×10⁷ per mouse when tumor volume reached 50-80 mm³. Each group consisted of 5 animals, which were dosed intravenously at the labeled dose once per day. Tumor volume after SI-38E17 administration is shown in the figure. At day 16, vehicle group tumor volume was 1298 mm³. All three doses of SI-38E17 had a significant tumor inhibition effect, with intermediate dose 0.005 mg (drug:TCR=12.5:1) having the best tumor inhibition (TGI=84%). Note that while all mice in the vehicle control group had died by day 22, tumor had been eliminated in 1 mouse (low dose), 3 mice (intermediate dose), and 1 mouse (high dose) by day 40. Thus, the tetra-GNC antibody, such as SI-38E17, shows strong tumor inhibition in vivo at multiple doses.

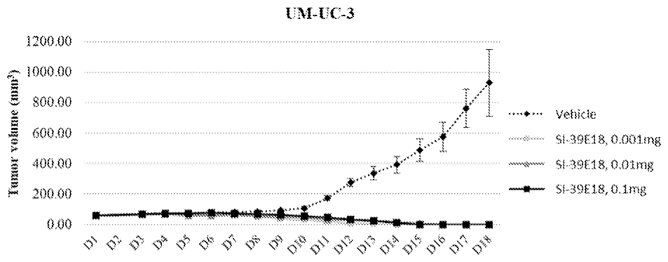

SI-39E18 was tested in a mouse xenograft model to examine its ability to slow tumor growth in vivo (FIG. 13). NCG mice were subcutaneously inoculated with 5×10⁶ human bladder cancer-derived UM-UC-3-EGFR VIII cells on the right flank. When the tumor grew to an average volume of 50-80 mm³, 5×10⁶ per mouse (100 ul) of human PBMC was injected in the abdominal cavity and different doses of SI-39E18 were given intravenously. Each group consisted of 5 animals, which were dosed intravenously at the labeled dose once per day for 18 total doses. The first day of dosing is defined as D1. The tumor growth after SI-39E18 administration is shown in the figure, which demonstrates that SI-39E18 elicits strong inhibition of tumor growth across multiple doses. As of the day of discontinuation (D18), the tumor volume of all dose groups (low dose group 0.001 mg, medium dose group 0.01 mg, and high dose group 0.1 mg) was 0 for three consecutive days, while that of the vehicle had increased significantly in size. Thus, the tetra-GNC antibody, such as SI-39E18, shows strong biological activity in vivo at multiple doses.

### Example 14. Exerting More Complete Cytotoxicity

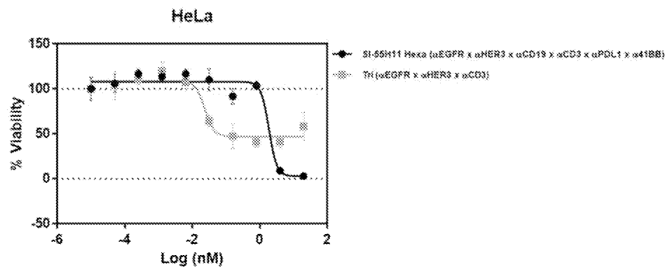

Hexa-GNC antibodies were created to explore multi-functionality as a single antibody therapeutics. SI-55H11 (SEQ ID NO. 53-56) is a hexa-GNC antibody having its binding specificities to CD3 (D1), EGFR (D2), PD-L1 (D3), 4-1BB (D4) on the heavy chain monomer, and HER3 (D5) and CD19 (D6) on its light chain moiety monomer (Table 1). The TDCC assay was used to determine the effect of the presence and absence of targeting PD-L1 and 4-1BB on T cell-mediated killing of tumor cells by comparing with a tri-specific antibody targeting CD3 (D1) on T cells and both EGFR (D2) and HER3 (D5) on tumor cells (Table 4). Serial dilutions (0 to 30 nM; 1 to 5 dilution factor) of Tri or Hexa GNC were added to a white 384-well plate containing luciferized BxPC3 (high EGFR expression) cells (plated 24 hours prior and grown at 37° C.) and activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. Data were fit to a sigmoidal function to calculate EC50 values and maximum killing (FIG. 14). The data shows that the hexa-GNC antibody exerts more complete killing than the tri-specific antibody (bottom plateau=46.92% viability; SI-55H11 bottom plateau=2.992% viability). The data implies that together with CD3 for T cell activation, simultaneously targeting immunomodulatory proteins, such as PD-L1 immune checkpoint and 4-1BB activation, is an effective combinational strategy for directing GNC response of immune system towards targeted cells and leading to more complete tumor depletion.

### Example 15. Targeting Multiple Tumor Antigens

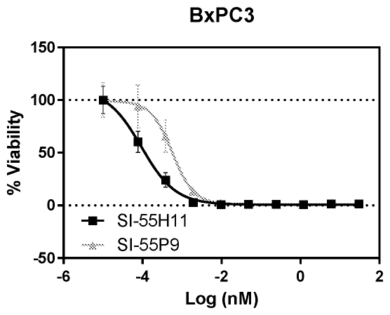

While the attempt to fix the immune targets on the heavy chain may be carried out as shown by Example 12 and 13, the binding domains on the light chain moiety may be dedicated to tumor-specific antigens (TSA), tumor-associated antigens (TAA), as well as neoantigens. In this context, several hexa-GNC antibodies were created and subjected to TDCC assay. The GNC antibodies were assessed to determine if additional tumor-targeting specificity can increase T cell-mediated killing of tumor cells (FIG. 15). Herein, SI-55P9 (SEQ ID NO. 33-36), a penta-GNC antibody capable of binding to EGFR, CD3, PD-L1, and 4-1BB via its heavy chain monomer, and CD19 via light chain moiety monomer, was compared to SI-55H11 (SEQ ID NO. 53-56), a hexa-GNC antibody with the same binding specificities plus the sixth specificity to HER3 via the light chain moiety monomer. Serial dilutions (0 to 30 nM; 1 to 5 dilution factor) of each GNC antibody were added to a white 384-well plate containing 500 luciferized BxPC3 (high EGFR; low HER3) cells (plated 24 hours prior and grown at 37° C.) and 2500 activated T cells (plated immediately before drug; effector:target=5:1) in a total volume of 50 ul. After an additional 72 hours, 20 ul of Bright-Glo (Promega) was added to wells, and luminescence corresponding to viability of luciferized tumor cells was determined using a CLARIOstar plate reader. Data were fit to a sigmoidal function to calculate EC50 values. The data shows that the hexa-GNC antibody displays higher potency in TDCC assay (SI-55P9 EC50=0.5727 pM; SI-55H11 EC50=0.09387 pM) when compared to the parental penta-GNC antibody lacking one of the tumor-targeting domains. Thus, an additional anti-tumor antigen binding domain can increase biological function of the GNC antibodies even against cells that express low levels of the TAA (in this case, HER3).

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. For example, while the above examples may include binding domains at certain positions, they are provided by way of comparison only and not by way of limitation. It is specifically contemplated by this application that the configuration of binding domains and their positions on the GNC proteins could be in any combination. As such, the illustrative embodiments of the present invention are not intended to be limited to the particular embodiments disclosed. Rather, they include all modifications and alternatives falling within the scope of the disclosure. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

### TABLES

TABLE 1
Configurations of example GNC antibodies and each binding domain in the structural
form of scFv (either V_{L} − V_{H} or V_{H} − V_{L} orientation, stapled or not), receptor, and ligand on heavy
chain (D1, D3, D4) and light chain moiety (D5, D6) (as described in FIG 1).

| GNC | Antibody | D1 | D2 | D3 | D4 | D5 | D6 |
|---|---|---|---|---|---|---|---|
| Penta- | SI-1P1 | αCD3^{a} (LH) | αEGFR | αPD-L1 (HL) | α4-1BB (HL) | αHER3 (LH) | — |
| | SI-1P2 | αCD3^{a} | αEGFR | αPD-L1 | α4-1BB | αHER3 | — |
| | | (LH Stapled) | | (HL Stapled) | (HL Stapled) | (LH Stapled) | |
| | SI-38P12 | αCD20 (LH) | αCD3^{a} | αPD-L1 (HL) | α4-1BB (HL) | — | αCD19^{d} (LH) |
| | SI-38P13 | αCD20 (LH) | αCD3^{c} | αPD-L1 (HL) | α4-1BB (HL) | αCD19^{d} (HL) | — |
| | SI-49P1 | αCD3^{b} | αMSLN | αPD-L1 (HL) | α4-1BB (HL) | NKG2D | — |
| | | (LH Stapled) | | | | | |
| | SI-49P3 | αCD3^{b} | αHER2 | αPD-L1 | α4-1BB | NKG2D | — |
| | | (LH Stapled) | | (HL Stapled) | (HL Stapled) | | |
| | SI-55P3 | Hu-αEGFR (LH) | αCD3^{c} | αPD-L1 (HL) | α4-1BB (HL) | — | αCD19^{d} (LH) |
| | SI-55P4 | Hu-αEGFR (LH) | αCD3^{c} | αPD-L1 (HL) | 4-1BBL trimer | — | αCD19^{d} (LH) |
| | SI-55P9 | Hu-αEGFR (LH) | αCD3^{a} | αPD-L1 (HL) | α4-1BB (HL) | — | αCD19^{d} (LH) |
| | SI-55P10 | Hu-αEGFR (LH) | αCD3^{a} | αPD-L1 (HL) | 4-1BBL trimer | — | αCD19^{d} (LH) |
| | SI-77P1 | αCD3^{b} (LH) | Hu-αEGFR | αPD-L1 (HL) | α4-1BB (HL) | — | αCD19^{e} (LH) |
| | SI-79P2 | Hu-αEGFR (LH) | αCD3^{c} | αPD-L1 (HL) | α4-1BB (HL) | — | αCD19^{d} (LH) |
| | SI-79P3 | Hu-αEGFR (LH) | αCD3^{c} | αPD-L1 (HL) | 4-1BBL trimer | — | αCD19^{d} (LH) |
| | SI-49P6 | αCD3^{b} | NKG2D | αPD-L1 | α4-1BB | — | αCD19^{f} (LH) |
| | | (LH Stapled) | | (HL Stapled) | (HL Stapled) | | |
| | SI-49P7 | αCD3^{b} | NKG2D | αPD-L1 | 4-1BBL trimer | — | αCD19^{f} (LH) |
| | | (LH Stapled) | | (HL Stapled) | | | |
| | SI-49P10 | αCD3^{b} | NKG2D | αPD-L1 | α4-1BB | — | αEGFR (LH) |
| | | (LH Stapled) | | (HL Stapled) | (HL Stapled) | | |
| Hexa- | SI-55H11 | Hu-αEGFR (LH) | αCD3^{a} | αPD-L1 (HL) | α4-1BB (HL) | αHER3 (LH) | αCD19^{e} (LH) |
| | | | (Stapled) | | | | |
| | SI-55H12 | Hu-αEGFR (LH) | αCD3^{a} | αPD-L1 (HL) | α4-1BB (HL) | αHER3 (LH) | αCD19^{e} (LH) |
| | SI-77H4 | αCD3^{b} (LH) | Hu-αEGFR | αPD-L1 (HL) | α4-1BB (HL) | αHER3 (LH) | αCD19^{e} (LH) |
| | | | (Stapled) | | | | |
| | SI-77H5 | αCD3^{b} (LH) | αEGFR | αPD-L1 (HL) | α4-1BB (HL) | αHER3 (LH) | αCD19^{e} (LH) |
| | | | (Stapled) | | | | |

^{a}284A10, see Applicant's application No. PCT/US2018/039143;
^{b}284A10 H1 (SEQ ID NO. 93-100), humanized anti-CD3 variable domain sequences encoding either a scFv domain or the Fab region, in either a “unstapled” or a “stapled” form (SEQ ID NO. 155-108); and
^{c}283E3 H1 (SEQ ID NO. 101-104), humanized anti-CD3 variable domain sequences encoding the Fab region.
^{d}SI-huBU12 VH (SEQ ID NO. 121, 122) and SI-huBU12 H1 VL (SEQ ID NO. 87, 88), humanized anti-CD19 variable domain sequences carrying R19S mutation.
^{e}SI-huBU12 H1 VH (SEQ ID NO. 85, 86) and SI-huBU12 H1 VL (SEQ ID NO. 87, 88), humanized anti-CD19 variable domain sequences.
^{f}SI-huBU12 VH (SEQ ID NO. 121, 122) and SI-huBU12 VL (SEQ ID NO. 131, 132), humanized anti-CD19 variable domain sequences carrying R19S mutation.

TABLE 2
The example penta-GNC antibodies.

| Protein | SI-1P1 | SI-1P2 | Tetra control |
|---|---|---|---|
| Format | Penta-GNC | Penta-GNC | Tetra-GNC |
| Specificity | EGFR × CD3 × | EGFR × CD3 × | EGFR × CD3 × |
| | PDL1 × 41BB × | PDL1 × 41BB × | PDL1 × 41BB |
| | HER3 | HER3 | |
| scFvs with | No | Yes | No |
| V_{H}-V_{L} | | | |
| disulfide bond | | | |
| HMW % | 2.27 | 3.48 | 2.38 |
| (time 0) | | | |
| HMW % | 2.56 | 2.81 | 2.75 |
| (2 weeks) | | | |
| HMW % | 2.35 | nd | 2.8 |
| (4 weeks) | | | |
| ΔHMW % after | 0.08 | −0.67 | 0.42 |
| 2 (or 4) weeks | | | |
| Melting | 67.84 | 69.35 | 59.89 |
| Temp (° C.) | | | |

HMW % was measured using preparative SEC; melting temperature was measuring using dynamic light scattering

TABLE 3
The kinetic parameters of example penta-
GNC antibody SI-1P1 versus SI-1P2,
where SI-1P2 contains disulfide bonds in
all four scFv domains (see Table 1).

| penta- | Human | | | Kon | Kdis |
|---|---|---|---|---|---|
| GNC | Ag | Response | KD (M) | (1/Ms) | (1/s) |
| SI-1P1 | CD3 | 0.1315 | 1.89E−08 | 3.41E+05 | 6.43E−03 |
| | ε/δ_His | | | | |
| | 4-1BB | 0.1768 | 9.97E−09 | 2.34E+05 | 2.33E−03 |
| | PD-L1 | 0.2332 | 3.44E−10 | 2.15E+05 | 7.37E−05 |
| | EGFR | 0.3544 | 4.61E−09 | 2.98E+05 | 1.37E−03 |
| | HER3 | 0.1927 | 1.77E−07 | 4.97E+04 | 8.79E−03 |
| SI-1P2 | CD3 | 0.1204 | 2.34E−08 | 2.81E+05 | 6.59E−03 |
| | ε/δ_His | | | | |
| | 4-1BB | 0.199 | 6.06E−09 | 3.68E+05 | 2.23E−03 |
| | PD-L1 | 0.2269 | 7.49E−10 | 3.51E+05 | 2.63E−04 |
| | EGFR | 0.4006 | 4.07E−09 | 3.56E+05 | 1.45E−03 |
| | HER3 | 0.1822 | 3.77E−07 | 7.71E+04 | 2.91E−02 |

TABLE 4
The affinity of each binding domain in example GNC antibodies

| GNC Format | Antibody | D1 K_{D} (nM) | D2 K_{D} (nM) | D3 K_{D} (nM) | D4 K_{D} (nM) | D5 K_{D} (nM) | D6 K_{D} (nM) |
|---|---|---|---|---|---|---|---|
| Tri-GNC | Control | CD3 (37.2) | EGFR (6.6) | — | — | HER3 (13.7) | — |
| | | Stapled | | | | | |
| Tetra-GNC | SI-35E20 | 4-1BB (4.03) | PD-L1 (0.379) | ROR1 (0.861) | CD3 (0.480) | — | — |
| | SI-38E17 | CD3 (23.5) | CD19 (1.48) | PD-L1 (0.524) | 4-1BB (8.19) | — | — |
| | SI-39E18 | CD3 (30.2) | EGFRvIII (16.4) | PD-L1 (0.421) | 4-1BB (18.1) | — | — |
| Penta-GNC | SI-1P1 | 18.9 | 4.61 | 0.344 | 9.97 | 177 | — |
| | SI-1P2 | 23.4 | 4.07 | 0.749 | 6.06 | 377 | — |
| | SI-38P12 | 744 | 12.5 | 0.195 | 4.85 | — | 1.11 |
| | SI-38P13 | 644 | 15.1 | 0.731 | 8.54 | 0.181 | — |
| | SI-49P3 | 4.42 | 0.029 | nq* | 2.75 | 1.39 | — |
| | SI-49P6 | nd** | 7.763*** | nd** | nd** | — | nd** |
| | SI-49P7 | nd** | 10.67*** | nd** | nd** | — | nd** |
| | SI-49P10 | 2.23 | 1.84 | nq* | 2.79 | — | nq* |
| | SI-55P9 | 7.41 | 20.8 | 0.407 | 7.55 | — | nq* |
| | SI-55P10 | 7.48 | 19.6 | 0.521 | 65.5 | — | nq* |
| Hexa-GNC | SI-55H11 | 4.65 | 15.2 | 0.737 | 4.91 | 9.23 | 6.80 |
| | SI-55H12 | 7.87 | 1.58 | 0.25 | 7.87 | 9.11 | nq* |

*Not quantified (strong binding was observed relative to reference sensor, but dissociation was too slow to quantify K_{D}).
**KD not determined.
***GNC protein as analyte, antigen as ligand.

TABLE 5
The levels of EGFR and HER3 expression
in the example cancer cell lines.

| Tumor cell Line | Description | EGFR | HER3 |
|---|---|---|---|
| BxPC3 | human pancreatic | High | Low |
| | cancer cells | | |
| MDA-MB-231 | human breast | High | Low |
| | cancer cells | | |
| Hela | human cervical | High | Low |
| | cancer cells | | |

TABLE 6
The example penta-GNC antibody targeting two tumor antigens in EGFR and HER3
displays higher potency in T cell activation and similar cytotoxicity as compared to its parental
control antibodies (see FIG. 2 and 3).

| GNC Antibody | | | | NFAT | TDCC | |
|---|---|---|---|---|---|---|
| | | | Tumor | BxPC3 | MDA-MB-231 | Hela |
| Name | GNC Format | Binding Specificity | Antigen | EC50 (nM) | EC50 (nM) | EC50 (nM) |
| SI-1P1 | penta- | CD3, EGFR, PD-L1, 4-1BB, HER3 | 2 | 1.46E−03 | 2.85E−05 | 1.03E−03 |
| Control | EGF-tetra- | CD3, EGFR, PD-L1, 4-1BB | 1 | 1.03E−03 | 3.13E−05 | 1.71E−03 |
| Control | FITC-tetra- | CD3, FITC, PD-L1, 4-1BB | None | 1.34E−02 | 1.23E−03 | n/a |
| Control | bispecific | EGFR, HER3 | Only | n/a | n/a | n/a |

TABLE 7
Characterization of example penta-GNC antibodies comprising a humanized anti-
EGFR scFv domain or Fab domain.

| GNC | αEGFR | Anti-EGFR | Specificities of Other | Titer | aSEC | huEGFR K_{D} | TDCC |
|---|---|---|---|---|---|---|---|
| Antibody | Variant | Domain | Binding Domains | (μg/ml) | % POI | (nM) | EC50 (pM) |
| SI-55P3 | H1 | scFv (D1) | αCD3 (2), αPD-L1 (3), | 175.9 | 86.13 | 18.1 | nd |
| | | | α4-1BB (4), αCD19 (6) | | | | |
| SI-55P4 | H1 | scFv (D1) | αCD3 (2), αPD-L1 (3), | 59.6 | 93.22 | nd | nd |
| | | | 41BBLT (4), αCD19 (6) | | | | |
| SI-79P2 | H4 | scFv (D1) | αCD3 (2), αPD-L1 (3), | 68.9 | 87.74 | 4.3 | nd |
| | | | α4-1BB (4), αCD19 (6) | | | | |
| SI-79P3 | H4 | scFv (D1) | αCD3 (2), αPD-L1 (3), | 70.3 | 92.8 | 5.21 | nd |
| | | | 41BBLT (4), αCD19 (6) | | | | |
| SI-55P9 | H7 | scFv (D1) | αCD3 (2), αPD-L1 (3), | 118 | 85.96 | 7.41 | 0.5727 |
| | | | α4-1BB (4), αCD19 (6) | | | | |
| SI-77P1 | H7 | Fab (D2) | αCD3 (1), αPD-L1 (3), | 98.1 | 77.94 | 2.17 | 0.0826 |
| | | | α4-1BB (4), αCD19 (6) | | | | |

TABLE 8
Characterization of example hexa-GNC antibodies comprising a humanized anti-
EGFR scFv domain or Fab region

| GNC | αEGFR | Anti-EGFR | Specificities of Other | Titer | aSEC | huEGFR | TDCC |
|---|---|---|---|---|---|---|---|
| Antibody | Variant | Domain | Binding domains | (μg/ml) | % POI | K_{D} (nM) | EC50 (pM) |
| SI-77H5 | C* | Fab (D2) | αCD3 (1), αPD-L1 (3), | 35 | 72.02 | 3.39 | nd |
| | | | α4-1BB (4), αHER3 (5), | | | | |
| | | | αCD19 (6) | | | | |
| SI-77H4 | H7 | Fab (D2) | αCD3 (1), αPD-L1 (3), | 61.1 | 77.25 | 3.29 | nd |
| | | | α4-1BB (4), αHER3 (5), | | | | |
| | | | αCD19 (6) | | | | |
| SI-55H11 | H7 | scFv (D1) | αCD3 (2), αPD-L1 (3), | 30 | 84.42 | 4.65 | 0.09387 |
| | | | α4-1BB (4), αHER3 (5), | | | | |
| | | | αCD19 (6) | | | | |

*Mouse sequences derived from Cetuximab.

SEQUENCE LISTING

| | | | SEQ ID NO. | |
|---|---|---|---|---|
| GNC | | Chain/ | Amino | Nucleo- |
| Protein | Name | Monomer | acid | tide |
| Penta- | SI-1P1 | H | 1 | 2 |
| | | L | 3 | 4 |
| | SI-1P2 | H | 5 | 6 |
| | | L | 7 | 8 |
| | SI-38P12 | H | 9 | 10 |
| | | L | 11 | 12 |
| | SI-38P13 | H | 13 | 14 |
| | | L | 15 | 16 |
| | SI-49P1 | H | 17 | 18 |
| | | L | 19 | 20 |
| | SI-49P3 | H | 21 | 22 |
| | | L | 23 | 24 |
| | SI-55P3 | H | 25 | 26 |
| | | L | 27 | 28 |
| | SI-55P4 | H | 29 | 30 |
| | | L | 31 | 32 |
| | SI-55P9 | H | 33 | 34 |
| | | L | 35 | 36 |
| | SI-55P10 | H | 37 | 38 |
| | | L | 39 | 40 |
| | SI-77P1 | H | 41 | 42 |
| | | L | 43 | 44 |
| | SI-79P2 | H | 45 | 46 |
| | | L | 47 | 48 |
| | SI-79P3 | H | 49 | 50 |
| | | L | 51 | 52 |
| | SI-49P10 | H | 117 | 118 |
| | | L | 119 | 120 |
| | SI-49P6 | H | 123 | 124 |
| | | L | 125 | 126 |
| | SI-49P7 | H | 127 | 128 |
| | | L | 129 | 130 |
| Hexa- | SI-55H11 | H | 53 | 54 |
| | | L | 55 | 56 |
| | SI-55H12 | H | 57 | 58 |
| | | L | 59 | 60 |
| | SI-77H4 | H | 61 | 62 |
| | | L | 63 | 64 |
| | SI-77H5 | H | 65 | 66 |
| | | L | 67 | 68 |
| Binding | αEGFR H1 | VH | 69 | 70 |
| Domain | | VL | 71 | 72 |
| | αEGFR H4 | VH | 73 | 74 |
| | | VL | 75 | 76 |
| | αEGFR H7 | VH | 77 | 78 |
| | | VL | 79 | 80 |
| | αEGFR H7 | VH | 81 | 82 |
| | stapled | VL | 83 | 84 |
| | αCD19 | VH | 121 | 122 |
| | SI-huBU12 | VL | 131 | 132 |
| | αCD19 SI- | VH | 85 | 86 |
| | huBU12 H1 | VL | 87 | 88 |
| | αCD3 284A10 | VH | 89 | 90 |
| | stapled | VL | 91 | 92 |
| | αCD3 | VH | 93 | 94 |
| | 284A10 H1 | VL | 95 | 96 |
| | αCD3 | VH | 97 | 98 |
| | 284A10 H1 | VL | 99 | 100 |
| | stapled | | | |
| | αCD3 | VH | 101 | 102 |
| | 283E3 H1 | VL | 103 | 104 |
| | αPD-L1 | VH | 105 | 106 |
| | PL221G5 | VL | 107 | 108 |
| | stapled | | | |
| | α41BB 466F6 | VH | 109 | 110 |
| | stapled | VL | 111 | 112 |
| | 4-1BB | — | 113 | 114 |
| | ligand trimer | | | |
| | NKG2D | — | 115 | 116 |
| | dimer | | | |

>Sequence ID 1: SI-1P1 heavy chain amino acid sequence
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGGGTKVEIKGGGGSGGGGSGGGGSG
GGGSEVQLVESGGGLVQPGGSLRLSCAASGFTISTNAMSWVRQAPGKGLEWIGVITGRDITYYA
SWAKGRFTISRDNSKNTLYLQMNSLRAEDTAVYYCARDGGSSAITSNNIWGQGTLVTVSTGGGG
SGGGGSQVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGAPSVFLFPPKPK
DTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQKS
LSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSEDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGGGTKVEIKGGGGSGGGGSGRSLVESGGGLVQPGGSLRLSCTASGFTISSYHMQWVRQAPG
KGLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGGGTKVEIK
>Sequence ID 2: SI-1P1 heavy chain nucleotide sequence
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAAGGCTATTTTTATTTTATTAGTCGTACTTATGTAAATTCTTTCGGCGGAGGGAC
CAAGGTGGAGATCAAAGGCGGTGGCGGTAGTGGGGGAGGCGGTTCTGGCGGCGGAGGGTCCGGC
GGTGGAGGATCAGAGGTGCAGCTGGTGGAGTCTGGGGGAGGCTTGGTCCAGCCTGGGGGGTCCC
TGAGACTCTCCTGTGCAGCCTCTGGATTCACCATCAGTACCAATGCAATGAGCTGGGTCCGCCA
GGCTCCAGGGAAGGGGCTGGAGTGGATCGGAGTCATTACTGGTCGTGATATCACATACTACGCG
AGCTGGGCGAAAGGCAGATTCACCATCTCCAGAGACAATTCCAAGAACACGCTGTATCTTCAAA
TGAACAGCCTGAGAGCCGAGGACACGGCTGTGTATTACTGTGCGAGAGACGGTGGTTCTTCTGC
TATTACTAGTAACAACATTTGGGGCCAGGGAACCCTGGTCACCGTGTCGACAGGCGGTGGAGGG
TCCGGCGGTGGTGGATCACAGGTGCAGCTGAAGCAGTCAGGACCTGGCCTAGTGCAGCCCTCAC
AGAGCCTGTCCATCACCTGCACAGTCTCTGGTTTCTCATTAACTAACTATGGTGTACACTGGGT
TCGCCAGTCTCCAGGAAAGGGTCTGGAGTGGCTGGGAGTGATATGGAGTGGTGGAAACACAGAC
TATAATACACCTTTCACATCCAGACTGAGCATCAACAAGGACAATTCCAAGAGCCAAGTTTTCT
TTAAAATGAACAGTCTGCAATCTAATGACACAGCCATATATTACTGTGCCAGAGCCCTCACCTA
CTATGATTACGAGTTTGCTTACTGGGGCCAAGGGACTCTGGTCACTGTCTCTAGCGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTCTCGAGCGGTGGAGGCGGATCTGGCGGAGGTGGTTCCGGCG
GTGGCGGCTCCGGTGGAGGCGGCTCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCGGAGGGACCAAGGTGGAGATCAAAGGCGGTGGAGGGTCCGGCGGTGGTGGCT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGCCAGGCTCCAGGG
AAGGGGCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTCAGGCCAGTCAGAACATTAGGACTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAATGA
>Sequence ID 3: SI-1P1 light chain moiety amino acid sequence
DILLTQSPVILSVSPGERVSFSCRASQSIGTNIHWYQQRTNGSPRLLIKYASESISGIPSRFSG
SGSGTDFTLSINSVESEDIADYYCQQNNNWPTTFGAGTKLELKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGECGGGGSGGGGSQSALTQPASVSGSPGQSITISCTGTSSDVGGY
NFVSWYQQHPGKAPKLMIYDVSDRPSGVSDRFSGSKSGNTASLIISGLQADDEADYYCSSYGSS
STHVIFGGGTKVTVLGGGGSGGGGSGGGGSGGGGSQVQLQESGGGLVKPGGSLRLSCAASGFTF
SSYWMSWVRQAPGKGLEWVANINRDGSASYYVDSVKGRFTISRDDAKNSLYLQMNSLRAEDTAV
YYCARDRGVGYFDLWGRGTLVTVSS
>Sequence ID 4: SI-1P1 light chain moiety nucleotide sequence
GACATCTTGCTGACTCAGTCTCCAGTCATCCTGTCTGTGAGTCCAGGAGAAAGAGTCAGTTTCT
CCTGCAGGGCCAGTCAGAGTATTGGCACAAACATACACTGGTATCAGCAAAGAACAAATGGTTC
TCCAAGGCTTCTCATAAAGTATGCTTCTGAGTCTATCTCTGGGATTCCTTCCAGGTTTAGTGGC
AGTGGATCAGGGACAGATTTTACTCTTAGCATCAACAGTGTGGAGTCTGAAGATATTGCAGATT
ATTACTGTCAACAAAATAATAACTGGCCAACCACGTTCGGTGCTGGGACCAAGCTGGAGCTGAA
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTGGCGGTGGAGGGTCCGGCGGTGGTGGATCCCAGTCTGCCCTGACTCAGCCTGCCTCCGTGTC
TGGGTCTCCTGGACAGTCGATCACCATCTCCTGCACTGGAACCAGCAGTGACGTTGGTGGTTAT
AACTTTGTCTCCTGGTACCAACAACACCCAGGCAAAGCCCCCAAACTCATGATCTATGATGTCA
GTGATCGGCCCTCAGGGGTGTCTGATCGCTTCTCCGGCTCCAAGTCTGGCAACACGGCCTCCCT
GATCATCTCTGGCCTCCAGGCTGACGACGAGGCTGATTATTACTGCAGCTCATATGGGAGCAGC
AGCACTCATGTGATTTTCGGCGGAGGGACCAAGGTGACCGTCCTAGGTGGAGGCGGTTCAGGCG
GAGGTGGTTCCGGCGGTGGCGGCTCCGGTGGAGGCGGCTCTCAGGTGCAATTGCAGGAGTCGGG
GGGAGGCCTGGTCAAGCCTGGAGGGTCCCTGAGACTCTCCTGTGCAGCCTCTGGATTCACCTTT
AGTAGTTATTGGATGAGCTGGGTCCGCCAGGCTCCAGGGAAGGGGCTGGAGTGGGTGGCCAACA
TAAACCGCGATGGAAGTGCGAGTTACTATGTGGACTCTGTGAAGGGCCGATTCACCATCTCCAG
AGACGACGCCAAGAACTCACTGTATCTGCAAATGAACAGCCTGAGAGCTGAGGACACGGCTGTG
TATTACTGTGCGAGAGATCGTGGGGTGGGCTACTTCGATCTCTGGGGCCGTGGCACCCTGGTCA
CCGTCTCGAGCTGA
>Sequence ID 5: SI-1P2 heavy chain amino acid sequence
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGCGTKVEIKGGGGSGGGGSGGGGSG
GGGSEVQLVESGGGLVQPGGSLRLSCAASGFTISTNAMSWVRQAPGKCLEWIGVITGRDITYYA
SWAKGRFTISRDNSKNTLYLQMNSLRAEDTAVYYCARDGGSSAITSNNIWGQGTLVTVSTGGGG
SGGGGSQVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGGPSVFLFPPKPK
DTLMISRTPEVTCVVVDVSHEDPEVKFNWYVDGVEVHNAKTKPREEQYASTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTOKS
LSLSPGGGGGSGGGGSEVQLLESGGGLVQPGGSLRLSCAASGFSFSSGYDMCWVRQAPGKCLEW
IACIAAGSAGITYDANWAKGRFTISRDNSKNTLYLQMNSLRAEDTAVYYCARSAFSFDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGCGTKVEIKGGGGSGGGGSGRSLVESGGGLVQPGGSLRLSCTASGFTISSYHMQWVRQAPG
KCLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGCGTKVEIK
>Sequence ID 6: SI-1P2 heavy chain nucleotide sequence
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAAGGCTATTTTTATTTTATTAGTCGTACTTATGTAAATTCTTTCGGCTGTGGGAC
CAAGGTGGAGATCAAAGGTGGAGGCGGTTCAGGCGGAGGTGGAAGTGGTGGTGGCGGCTCTGGA
GGCGGCGGATCTGAGGTGCAGCTGGTGGAGTCTGGGGGAGGCTTGGTCCAGCCTGGGGGGTCCC
TGAGACTCTCCTGTGCAGCCTCTGGATTCACCATCAGTACCAATGCAATGAGCTGGGTCCGCCA
GGCTCCAGGGAAGTGCCTGGAGTGGATCGGAGTCATTACTGGTCGTGATATCACATACTACGCG
AGCTGGGCGAAAGGCAGATTCACCATCTCCAGAGACAATTCCAAGAACACGCTGTATCTTCAAA
TGAACAGCCTGAGAGCCGAGGACACGGCTGTGTATTACTGTGCGAGAGACGGTGGTTCTTCTGC
TATTACTAGTAACAACATTTGGGGCCAGGGAACCCTGGTCACCGTGTCGACAGGCGGTGGAGGG
TCCGGCGGTGGTGGATCACAGGTGCAGCTGAAGCAGTCAGGACCTGGCCTAGTGCAGCCCTCAC
AGAGCCTGTCCATCACCTGCACAGTCTCTGGTTTCTCATTAACTAACTATGGTGTACACTGGGT
TCGCCAGTCTCCAGGAAAGGGTCTGGAGTGGCTGGGAGTGATATGGAGTGGTGGAAACACAGAC
TATAATACACCTTTCACATCCAGACTGAGCATCAACAAGGACAATTCCAAGAGCCAAGTTTTCT
TTAAAATGAACAGTCTGCAATCTAATGACACAGCCATATATTACTGTGCCAGAGCCCTCACCTA
CTATGATTACGAGTTTGCTTACTGGGGCCAAGGGACTCTGGTCACTGTCTCTAGCGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACGCCAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGTGCCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTCTCGAGCGGTGGAGGCGGTTCAGGCGGAGGTGGAAGTGGTG
GTGGCGGCTCTGGAGGCGGCGGATCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCTGCGGGACCAAGGTGGAGATCAAAGGTGGTGGCGGCTCTGGAGGAGGAGGGT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGGCAGGCACCTGGG
AAGTGCCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTCAGGCCAGTCAGAACATTAGGACTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CTTGGAACCTGGCGATGCTGCAACTTACTATTGTCAGTCTACCTATCTTGGTACTGATTATGTT
GGCGGTGCTTTCGGCTGTGGGACCAAGGTGGAGATCAAATGA
>Sequence ID 7: SI-1P2 light chain moiety amino acid sequence
DILLTQSPVILSVSPGERVSFSCRASQSIGTNIHWYQQRTNGSPRLLIKYASESISGIPSRFSG
SGSGTDFTLSINSVESEDIADYYCQQNNNWPTTFGAGTKLELKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGECGGGGSGGGGSQSALTQPASVSGSPGQSITISCTGTSSDVGGY
NFVSWYQQHPGKAPKLMIYDVSDRPSGVSDRFSGSKSGNTASLIISGLQADDEADYYCSSYGSS
STHVIFGCGTKVTVLGGGGSGGGGSGGGGSGGGGSQVQLQESGGGLVKPGGSLRLSCAASGFTF
SSYWMSWVRQAPGKCLEWVANINRDGSASYYVDSVKGRFTISRDDAKNSLYLQMNSLRAEDTAV
YYCARDRGVGYFDLWGRGTLVTVSS
>Sequence ID 8: SI-1P2 light chain moiety nucleotide sequence
GACATCTTGCTGACTCAGTCTCCAGTCATCCTGTCTGTGAGTCCAGGAGAAAGAGTCAGTTTCT
CCTGCAGGGCCAGTCAGAGTATTGGCACAAACATACACTGGTATCAGCAAAGAACAAATGGTTC
TCCAAGGCTTCTCATAAAGTATGCTTCTGAGTCTATCTCTGGGATTCCTTCCAGGTTTAGTGGC
AGTGGATCAGGGACAGATTTTACTCTTAGCATCAACAGTGTGGAGTCTGAAGATATTGCAGATT
ATTACTGTCAACAAAATAATAACTGGCCAACCACGTTCGGTGCTGGGACCAAGCTGGAGCTGAA
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTGGCGGTGGAGGGTCCGGCGGTGGTGGATCCCAGTCTGCCCTGACTCAGCCTGCCTCCGTGTC
TGGGTCTCCTGGACAGTCGATCACCATCTCCTGCACTGGAACCAGCAGTGACGTTGGTGGTTAT
AACTTTGTCTCCTGGTACCAACAACACCCAGGCAAAGCCCCCAAACTCATGATCTATGATGTCA
GTGATCGGCCCTCAGGGGTGTCTGATCGCTTCTCCGGCTCCAAGTCTGGCAACACGGCCTCCCT
GATCATCTCTGGCCTCCAGGCTGACGACGAGGCTGATTATTACTGCAGCTCATATGGGAGCAGC
AGCACTCATGTGATTTTCGGCTGCGGGACCAAGGTGACCGTCCTAGGTGGAGGCGGTTCAGGCG
GAGGTGGTTCCGGCGGTGGCGGCTCCGGTGGAGGCGGCTCTCAGGTGCAATTGCAGGAGTCGGG
GGGAGGCCTGGTCAAGCCTGGAGGGTCCCTGAGACTCTCCTGTGCAGCCTCTGGATTCACCTTT
AGTAGTTATTGGATGAGCTGGGTCCGCCAGGCTCCAGGGAAGTGCCTGGAGTGGGTGGCCAACA
TAAACCGCGATGGAAGTGCGAGTTACTATGTGGACTCTGTGAAGGGCCGATTCACCATCTCCAG
AGACGACGCCAAGAACTCACTGTATCTGCAAATGAACAGCCTGAGAGCTGAGGACACGGCTGTG
TATTACTGTGCGAGAGATCGTGGGGTGGGCTACTTCGATCTCTGGGGCCGTGGCACCCTGGTCA
CCGTCTCGAGCTGA
>Sequence ID 9: SI-38P12 heavy chain amino acid sequence
QIVLSQSPAILSASPGEKVTMTCRASSSVSYIHWFQQKPGSSPKPWIYATSNLASGVPVRFSGS
GSGTSYSLTISRVEAEDAATYYCQQWTSNPPTFGGGTKLTVLGGGGSGGGGSGGGGSGGGGSQV
QLQQPGAELVKPGASVKMSCKASGYTFTSYNMHWVKQTPGRGLEWIGAIYPGNGDTSYNQKFKG
KATLTADKSSSTAYMQLSSLTSEDSAVYYCARSTYYGGDWYFNVWGAGTTVTVSSGGGGSGGGG
SEVQLVESGGGLVQPGGSLRLSCAASGFTISTNAMSWVRQAPGKGLEWIGVITGRDITYYASWA
KGRFTISRDNSKNTLYLQMNSLRAEDTAVYYCARDGGSSAITSNNIWGQGTLVTVSSASTKGPS
VFPLAPSSKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPEAAGAPSVFLFPPKPKDTLM
ISRTPEVTCVVVDVSHEDPEVKFNWYVDGVEVHNAKTKPREEQYNSTYRVVSVLTVLHQDWLNG
KEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLS
PGGGGGSGGGGSEVQLLESGGGLVQPGGSLRLSCAASGFSFSSGYDMCWVRQAPGKGLEWIACI
AAGSAGITYDANWAKGRFTISRDNSKNTLYLQMNSLRAEDTAVYYCARSAFSFDYAMDLWGQGT
LVTVSSGGGGSGGGGSGGGGSGGGGSDIQMTQSPSTLSASVGDRVTITCQASQSISSHLNWYQQ
KPGKAPKLLIYKASTLASGVPSRFSGSGSGTEFTLTISSLQPDDFATYYCQQGYSWGNVDNVFG
GGTKVEIKGGGGSGGGGSGRSLVESGGGLVQPGGSLRLSCTASGFTISSYHMQWVRQAPGKGLE
YIGTISSGGNVYYASSARGRFTISRPSSKNTVDLQMNSLRAEDTAVYYCARDSGYSDPMWGQGT
LVTVSSGGGGSGGGGSGGGGSGGGGSDVVMTQSPSSVSASVGDRVTITCQASQNIRTYLSWYQQ
KPGKAPKLLIYAAANLASGVPSRFSGSGSGTDFTLTISDLEPGDAATYYCQSTYLGTDYVGGAF
GGGTKVEIK
>Sequence ID 10: SI-38P12 heavy chain nucleotide sequence
CAGATCGTGCTGAGCCAGAGCCCCGCCATCCTGAGCGCCAGCCCCGGCGAGAAGGTGACCATGA
CCTGCCGGGCCAGCAGCAGCGTGAGCTACATCCACTGGTTCCAGCAGAAGCCCGGCAGCAGCCC
CAAGCCCTGGATCTACGCCACCAGCAACCTGGCCAGCGGCGTGCCCGTGCGGTTCAGCGGCAGC
GGCAGCGGCACCAGCTACAGCCTGACCATCAGCCGGGTGGAGGCCGAGGACGCCGCCACCTACT
ACTGCCAGCAGTGGACCAGCAACCCCCCCACCTTCGGCGGCGGCACCAAGCTGACCGTGCTGGG
TGGTGGTGGCTCTGGAGGAGGCGGGAGCGGGGGTGGTGGCTCAGGTGGTGGAGGTTCCCAGGTG
CAGCTGCAGCAGCCCGGCGCCGAGCTGGTGAAGCCCGGCGCCAGCGTGAAGATGAGCTGCAAGG
CCAGCGGCTACACCTTCACCAGCTACAACATGCACTGGGTGAAGCAGACCCCCGGCCGGGGCCT
GGAGTGGATCGGCGCCATCTACCCCGGCAACGGCGACACCAGCTACAACCAGAAGTTCAAGGGC
AAGGCCACCCTGACCGCCGACAAGAGCAGCAGCACCGCCTACATGCAGCTGAGCAGCCTGACCA
GCGAGGACAGCGCCGTGTACTACTGCGCCCGGAGCACCTACTACGGCGGCGACTGGTACTTCAA
CGTGTGGGGCGCCGGCACCACCGTGACCGTCTCGAGTGGCGGTGGAGGGTCCGGCGGTGGTGGA
TCAGAGGTGCAGCTGGTGGAGTCTGGGGGAGGCTTGGTCCAGCCTGGGGGGTCCCTGAGACTCT
CCTGTGCAGCCTCTGGATTCACCATCAGTACCAATGCAATGAGCTGGGTCCGCCAGGCTCCAGG
GAAGGGGCTGGAGTGGATCGGAGTCATTACTGGTCGTGATATCACATACTACGCGAGCTGGGCG
AAAGGCAGATTCACCATCTCCAGAGACAATTCCAAGAACACGCTGTATCTTCAAATGAACAGCC
TGAGAGCCGAGGACACGGCTGTGTATTACTGTGCGAGAGACGGTGGTTCTTCTGCTATTACTAG
TAACAACATTTGGGGCCAGGGAACCCTGGTCACCGTGTCCTCAGCTAGCACCAAGGGCCCATCG
GTCTTCCCCCTGGCACCCTCCTCCAAGAGCACCTCTGGGGGCACAGCGGCCCTGGGCTGCCTGG
TCAAGGACTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACCCAGACCTACATCTGCAACGTGAATCACAAGCCCAGCAACACCA
AGGTGGACAAGAGAGTTGAGCCCAAATCTTGTGACAAAACTCACACATGCCCACCGTGCCCAGC
ACCTGAAGCCGCGGGGGCACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGGC
AAGGAGTACAAGTGCGCGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTT
CTCATGCTCCGTGATGCATGAGGCTCTGCACAACCACTACACGCAGAAGAGCCTCTCCCTGTCT
CCGGGTGGCGGTGGAGGGTCCGGCGGTGGTGGATCCGAGGTGCAGCTGTTGGAGTCTGGGGGAG
GCTTGGTACAGCCTGGGGGGTCCCTGAGACTCTCCTGTGCAGCCTCTGGATTCTCCTTCAGTAG
CGGGTACGACATGTGCTGGGTCCGCCAGGCTCCAGGGAAGGGGCTGGAGTGGATCGCATGCATT
GCTGCTGGTAGTGCTGGTATCACTTACGACGCGAACTGGGCGAAAGGCCGGTTCACCATCTCCA
GAGACAATTCCAAGAACACGCTGTATCTGCAAATGAACAGCCTGAGAGCCGAGGACACGGCCGT
ATATTACTGTGCGAGATCGGCGTTTTCGTTCGACTACGCCATGGACCTCTGGGGCCAGGGAACC
CTGGTCACCGTGTCGAGCGGTGGAGGCGGATCTGGCGGAGGTGGTTCCGGCGGTGGCGGCTCCG
GTGGAGGCGGCTCTGACATCCAGATGACCCAGTCTCCTTCCACCCTGTCTGCATCTGTAGGAGA
CAGAGTCACCATCACTTGCCAGGCCAGTCAGAGCATTAGTTCCCACTTAAACTGGTATCAGCAG
AAACCAGGGAAAGCCCCTAAGCTCCTGATCTATAAGGCATCCACTCTGGCATCTGGGGTCCCAT
CAAGGTTCAGCGGCAGTGGATCTGGGACAGAATTTACTCTCACCATCAGCAGCCTGCAGCCTGA
TGATTTTGCAACTTATTACTGCCAACAGGGTTATAGTTGGGGTAATGTTGATAATGTTTTCGGC
GGAGGGACCAAGGTGGAGATCAAAGGCGGTGGAGGGTCCGGCGGTGGTGGCTCCGGACGGTCGC
TGGTGGAGTCTGGGGGAGGCTTGGTCCAGCCTGGGGGGTCCCTGAGACTCTCCTGTACTGCCTC
TGGATTCACCATCAGTAGCTACCACATGCAGTGGGTCCGCCAGGCTCCAGGGAAGGGGCTGGAG
TACATCGGAACCATTAGTAGTGGTGGTAATGTATACTACGCAAGCTCCGCTAGAGGCAGATTCA
CCATCTCCAGACCCTCGTCCAAGAACACGGTGGATCTTCAAATGAACAGCCTGAGAGCCGAGGA
CACGGCTGTGTATTACTGTGCGAGAGACTCTGGTTATAGTGATCCTATGTGGGGCCAGGGAACC
CTGGTCACCGTCTCTTCAGGCGGTGGCGGTAGTGGGGGAGGCGGTTCTGGCGGCGGAGGGTCCG
GCGGTGGAGGATCAGACGTTGTGATGACCCAGTCTCCATCTTCCGTGTCTGCATCTGTAGGAGA
CAGAGTCACCATCACCTGTCAGGCCAGTCAGAACATTAGGACTTACTTATCCTGGTATCAGCAG
AAACCAGGGAAAGCCCCTAAGCTCCTGATCTATGCTGCAGCCAATCTGGCATCTGGGGTCCCAT
CAAGGTTCAGCGGCAGTGGATCTGGGACAGATTTCACTCTCACCATCAGCGACCTGGAGCCTGG
CGATGCTGCAACTTACTATTGTCAGTCTACCTATCTTGGTACTGATTATGTTGGCGGTGCTTTC
GGCGGAGGGACCAAGGTGGAGATCAAATGA
>Sequence ID 11: SI-38P12 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGGGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC
>Sequence ID 12: SI-38P12 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGCGGCGGTGGAGGGTCCGGCGGTGGTGGATCA
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTCACTCTCACCATCAGCAGCCTGCAGCCTGATGATTTTGCAACTT
ATTACTGCCAAGGCTATTTTTATTTTATTAGTCGTACTTATGTAAATTCTTTCGGCGGAGGGAC
CAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTTGA
>Sequence ID 13: SI-38P13 heavy chain amino acid sequence
QIVLSQSPAILSASPGEKVTMTCRASSSVSYIHWFQQKPGSSPKPWIYATSNLASGVPVRFSGS
GSGTSYSLTISRVEAEDAATYYCQQWTSNPPTFGGGTKLTVLGGGGSGGGGSGGGGSGGGGSQV
QLQQPGAELVKPGASVKMSCKASGYTFTSYNMHWVKQTPGRGLEWIGAIYPGNGDTSYNQKFKG
KATLTADKSSSTAYMQLSSLTSEDSAVYYCARSTYYGGDWYFNVWGAGTTVTVSSGGGGSGGGG
SQVQLQESGGRLVQPGEPLSLTCKTSGIDLSSNAIGWVRQAPGKGLEWIGVIFGSGNTYYASWA
KGRFTISRSTSTVYLKMNSLRSEDTAIYYCARGGYSSDIWGQGTLVTVSSASTKGPSVFPLAPS
SKSTSGGTAALGCLVKDYFPEPVTVSWNSGALTSGVHTFPAVLQSSGLYSLSSVVTVPSSSLGT
QTYICNVNHKPSNTKVDKRVEPKSCDKTHTCPPCPAPEAAGAPSVFLFPPKPKDTLMISRTPEV
TCVVVDVSHEDPEVKFNWYVDGVEVHNAKTKPREEQYNSTYRVVSVLTVLHQDWLNGKEYKCAV
SNKALPAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTOKSLSLSPGGGGGS
GGGGSEVQLLESGGGLVQPGGSLRLSCAASGFSFSSGYDMCWVRQAPGKGLEWIACIAAGSAGI
TYDANWAKGRFTISRDNSKNTLYLQMNSLRAEDTAVYYCARSAFSFDYAMDLWGQGTLVTVSSG
GGGSGGGGSGGGGSGGGGSDIQMTQSPSTLSASVGDRVTITCQASQSISSHLNWYQQKPGKAPK
LLIYKASTLASGVPSRFSGSGSGTEFTLTISSLQPDDFATYYCQQGYSWGNVDNVFGGGTKVEI
KGGGGSGGGGSGRSLVESGGGLVQPGGSLRLSCTASGFTISSYHMQWVRQAPGKGLEYIGTISS
GGNVYYASSARGRFTISRPSSKNTVDLQMNSLRAEDTAVYYCARDSGYSDPMWGQGTLVTVSSG
GGGSGGGGSGGGGSGGGGSDVVMTQSPSSVSASVGDRVTITCQASQNIRTYLSWYQQKPGKAPK
LLIYAAANLASGVPSRFSGSGSGTDFTLTISDLEPGDAATYYCQSTYLGTDYVGGAFGGGTKVE
IK
>Sequence ID 14: SI-38P13 heavy chain nucleotide sequence
CAGATCGTGCTGAGCCAGAGCCCCGCCATCCTGAGCGCCAGCCCCGGCGAGAAGGTGACCATGA
CCTGCCGGGCCAGCAGCAGCGTGAGCTACATCCACTGGTTCCAGCAGAAGCCCGGCAGCAGCCC
CAAGCCCTGGATCTACGCCACCAGCAACCTGGCCAGCGGCGTGCCCGTGCGGTTCAGCGGCAGC
GGCAGCGGCACCAGCTACAGCCTGACCATCAGCCGGGTGGAGGCCGAGGACGCCGCCACCTACT
ACTGCCAGCAGTGGACCAGCAACCCCCCCACCTTCGGCGGCGGCACCAAGCTGACCGTGCTGGG
TGGTGGTGGCTCTGGAGGAGGCGGGAGCGGGGGTGGTGGCTCAGGTGGTGGAGGTTCCCAGGTG
CAGCTGCAGCAGCCCGGCGCCGAGCTGGTGAAGCCCGGCGCCAGCGTGAAGATGAGCTGCAAGG
CCAGCGGCTACACCTTCACCAGCTACAACATGCACTGGGTGAAGCAGACCCCCGGCCGGGGCCT
GGAGTGGATCGGCGCCATCTACCCCGGCAACGGCGACACCAGCTACAACCAGAAGTTCAAGGGC
AAGGCCACCCTGACCGCCGACAAGAGCAGCAGCACCGCCTACATGCAGCTGAGCAGCCTGACCA
GCGAGGACAGCGCCGTGTACTACTGCGCCCGGAGCACCTACTACGGCGGCGACTGGTACTTCAA
CGTGTGGGGCGCCGGCACCACCGTGACCGTCTCGAGTGGCGGTGGAGGGTCCGGCGGTGGTGGA
TCACAAGTGCAGTTGCAAGAAAGTGGTGGTAGACTGGTTCAGCCTGGTGAACCCTTGTCACTGA
CGTGTAAAACAAGCGGCATTGATCTGTCCTCTAACGCCATCGGATGGGTCCGACAGGCCCCAGG
AAAAGGTCTGGAGTGGATCGGAGTTATCTTCGGGAGCGGCAATACATACTACGCAAGCTGGGCA
AAAGGGCGATTTACGATATCACGGAGCACCTCTACAGTTTATTTGAAAATGAACTCCCTCCGGT
CCGAGGATACCGCGATATATTACTGTGCCAGAGGGGGGTACTCCTCTGATATCTGGGGGCAGGG
TACACTGGTTACAGTTTCATCCGCTAGCACCAAGGGCCCATCGGTCTTCCCCCTGGCACCCTCC
TCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAGAGTTGAGC
CCAAATCTTGTGACAAAACTCACACATGCCCACCGTGCCCAGCACCTGAAGCCGCGGGGGCACC
GTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCGCGGTC
TCCAACAAAGCCCTCCCAGCCCCCATCGAGAAAACCATCTCCAAAGCCAAAGGGCAGCCCCGAG
AACCACAGGTGTACACCCTGCCCCCATCCCGGGATGAGCTGACCAAGAACCAGGTCAGCCTGAC
CTGCCTGGTCAAAGGCTTCTATCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGA
GGCTCTGCACAACCACTACACGCAGAAGAGCCTCTCCCTGTCTCCGGGTGGCGGTGGAGGGTCC
GGCGGTGGTGGATCCGAGGTGCAGCTGTTGGAGTCTGGGGGAGGCTTGGTACAGCCTGGGGGGT
CCCTGAGACTCTCCTGTGCAGCCTCTGGATTCTCCTTCAGTAGCGGGTACGACATGTGCTGGGT
CCGCCAGGCTCCAGGGAAGGGGCTGGAGTGGATCGCATGCATTGCTGCTGGTAGTGCTGGTATC
ACTTACGACGCGAACTGGGCGAAAGGCCGGTTCACCATCTCCAGAGACAATTCCAAGAACACGC
TGTATCTGCAAATGAACAGCCTGAGAGCCGAGGACACGGCCGTATATTACTGTGCGAGATCGGC
GTTTTCGTTCGACTACGCCATGGACCTCTGGGGCCAGGGAACCCTGGTCACCGTGTCGAGCGGT
GGAGGCGGATCTGGCGGAGGTGGTTCCGGCGGTGGCGGCTCCGGTGGAGGCGGCTCTGACATCC
AGATGACCCAGTCTCCTTCCACCCTGTCTGCATCTGTAGGAGACAGAGTCACCATCACTTGCCA
GGCCAGTCAGAGCATTAGTTCCCACTTAAACTGGTATCAGCAGAAACCAGGGAAAGCCCCTAAG
CTCCTGATCTATAAGGCATCCACTCTGGCATCTGGGGTCCCATCAAGGTTCAGCGGCAGTGGAT
CTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATC
AAAGGCGGTGGAGGGTCCGGCGGTGGTGGCTCCGGACGGTCGCTGGTGGAGTCTGGGGGAGGCT
TGGTCCAGCCTGGGGGGTCCCTGAGACTCTCCTGTACTGCCTCTGGATTCACCATCAGTAGCTA
CCACATGCAGTGGGTCCGCCAGGCTCCAGGGAAGGGGCTGGAGTACATCGGAACCATTAGTAGT
GGTGGTAATGTATACTACGCAAGCTCCGCTAGAGGCAGATTCACCATCTCCAGACCCTCGTCCA
AGAACACGGTGGATCTTCAAATGAACAGCCTGAGAGCCGAGGACACGGCTGTGTATTACTGTGC
GAGAGACTCTGGTTATAGTGATCCTATGTGGGGCCAGGGAACCCTGGTCACCGTCTCTTCAGGC
GGTGGCGGTAGTGGGGGAGGCGGTTCTGGCGGCGGAGGGTCCGGCGGTGGAGGATCAGACGTTG
TGATGACCCAGTCTCCATCTTCCGTGTCTGCATCTGTAGGAGACAGAGTCACCATCACCTGTCA
GGCCAGTCAGAACATTAGGACTTACTTATCCTGGTATCAGCAGAAACCAGGGAAAGCCCCTAAG
CTCCTGATCTATGCTGCAGCCAATCTGGCATCTGGGGTCCCATCAAGGTTCAGCGGCAGTGGAT
CTGGGACAGATTTCACTCTCACCATCAGCGACCTGGAGCCTGGCGATGCTGCAACTTACTATTG
TCAGTCTACCTATCTTGGTACTGATTATGTTGGCGGTGCTTTCGGCGGAGGGACCAAGGTGGAG
ATCAAATGA
>Sequence ID 15: SI-38P13 light chain moiety amino acid sequence
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGECGGGGSGGGGSGGGGSQVTLKESGPGLVQPGQTLRLTCAF
SGFSLSTSGMGVGWIRQPPGKGLEWLAHIWWDDDKRYNPALKSRLTISKDTSKNQVYLQMNSLD
AEDTAVYYCARMELWSYYFDYWGQGTLVTVSSGGGGSGGGGSGGGGSGGGGSENVLTQSPASLS
ASPGERVTITCSASSSVSYMHWYQQKPGQAPKLWIYDTSKLASGVPSRFSGSGSGNDHTLTISS
MEPEDFATYYCFQGSVYPFTFGQGTKVTVL
>Sequence ID 16: SI-38P13 light chain moiety nucleotide sequence
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAGCGTACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACA
GGGGAGAGTGTGGTGGAGGCGGTTCAGGCGGAGGTGGTTCCGGCGGTGGCGGCTCCCAGGTCAC
ATTGAAGGAATCTGGCCCCGGCCTTGTTCAGCCAGGACAGACCCTTAGGCTCACCTGTGCCTTC
AGTGGTTTTTCTCTTAGCACTAGCGGTATGGGGGTCGGCTGGATTCGGCAGCCTCCCGGCAAAG
GTCTTGAGTGGTTGGCTCACATTTGGTGGGACGACGACAAACGGTATAATCCTGCCTTGAAAAG
TCGGCTGACCATTAGTAAGGATACCTCAAAAAATCAAGTGTACTTGCAAATGAATAGCCTTGAC
GCCGAGGATACGGCTGTATATTATTGCGCGCGGATGGAACTCTGGTCTTACTACTTTGATTATT
GGGGGCAGGGGACTCTCGTCACGGTCTCGAGTGGTGGAGGCGGTTCAGGCGGAGGTGGTTCCGG
CGGTGGCGGCTCCGGTGGAGGCGGCTCTGAAAATGTATTGACACAGAGCCCCGCCTCCCTCAGT
GCCTCACCTGGGGAAAGGGTAACTATCACTTGCTCTGCATCAAGCAGCGTCTCATACATGCATT
GGTATCAACAAAAGCCTGGACAGGCCCCCAAGCTCTGGATATACGATACGAGCAAGCTGGCTTC
CGGCGTACCTAGCCGCTTCAGTGGTTCCGGCTCAGGCAACGATCACACCCTTACGATTTCCAGT
ATGGAACCCGAAGATTTTGCAACTTATTATTGTTTCCAGGGGAGCGTGTACCCATTCACTTTCG
GGCAGGGGACAAAAGTGACGGTACTGTGA
>Sequence ID 17: SI-49P1 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVLGGGGSGGGGSGGGGSG
GGGSEVQLVESGGGLVQPGGSLRLSCTASGFTISTNAMSWVRQAPGKCLEWVGVITGRDITYYA
SWAKGRFTISRDTSKNTVYLQMNSLRAEDTAVYYCARDGGSSAITSNNIWGQGTLVTVSTGGGG
SGGGGSQVQLQQSGPELEKPGASVKISCKASGYSFTGYTMNWVKQSHGKSLEWIGLITPYNGAS
SYNQKFRGKATLTVDKSSSTAYMDLLSLTSEDSAVYFCARGGYDGRGFDYWGSGTPVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGGPSVFLFPPKPK
DTLMISRTPEVTCVVVDVSHEDPEVKFNWYVDGVEVHNAKTKPREEQYASTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQKS
LSLSPGGGGGSGGGGSEVQLLESGGGLVQPGGSLRLSCAASGFSFSSGYDMCWVRQAPGKCLEW
IACIAAGSAGITYDANWAKGRFTISRDNSKNTLYLQMNSLRAEDTAVYYCARSAFSFDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGCGTKVEIKGGGGSGGGGSGRSLVESGGGLVQPGGSLRLSCTASGFTISSYHMQWVRQAPG
KCLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGCGTKVEIK
>Sequence ID 18: SI-49P1 heavy chain nucleotide sequence
GAGATCGTGATGACCCAGAGCCCCAGCACCCTGAGCGCCAGCGTGGGCGACAGGGTGATCATCA
CCTGCCAGGCCAGCGAGAGCATCAGCAGCTGGCTGGCCTGGTACCAGCAGAAGCCCGGCAAGGC
CCCCAAGCTGCTGATCTACGAGGCCAGCAAGCTGGCCAGCGGCGTGCCCAGCAGGTTCAGCGGC
AGCGGCAGCGGCGCCGAGTTCACCCTGACCATCAGCAGCCTGCAGCCCGACGACTTCGCCACCT
ACTACTGCCAGGGCTACTTCTACTTCATCAGCAGGACCTACGTGAACAGCTTCGGCTGCGGCAC
CAAGCTGACCGTGCTGGGCGGTGGCGGTAGTGGGGGAGGCGGTTCTGGCGGCGGAGGGTCCGGC
GGTGGAGGATCAGAGGTGCAGCTGGTGGAGAGCGGCGGCGGCCTGGTGCAGCCCGGCGGCAGCC
TGAGGCTGAGCTGCACCGCCAGCGGCTTCACCATCAGCACCAACGCCATGAGCTGGGTGAGGCA
GGCCCCCGGCAAGTGCCTGGAGTGGGTGGGCGTGATCACCGGCAGGGACATCACCTACTACGCC
AGCTGGGCCAAGGGCAGGTTCACCATCAGCAGGGACACCAGCAAGAACACCGTGTACCTGCAGA
TGAACAGCCTGAGGGCCGAGGACACCGCCGTGTACTACTGCGCCAGGGACGGCGGCAGCAGCGC
CATCACCAGCAACAACATCTGGGGCCAGGGCACCCTGGTGACCGTGTCGACAGGCGGTGGAGGG
TCCGGCGGTGGTGGATCACAGGTACAACTGCAGCAGTCTGGGCCTGAGCTGGAGAAGCCTGGCG
CTTCAGTGAAGATATCCTGCAAGGCTTCTGGTTACTCATTCACTGGCTACACCATGAACTGGGT
GAAGCAGAGCCATGGAAAGAGCCTTGAGTGGATTGGACTTATTACTCCTTACAATGGTGCTTCT
AGCTACAACCAGAAGTTCAGGGGCAAGGCCACATTAACTGTAGACAAGTCATCCAGCACAGCCT
ACATGGACCTCCTCAGTCTGACATCTGAAGACTCTGCAGTCTATTTCTGTGCAAGGGGGGGTTA
CGACGGGAGGGGTTTTGACTACTGGGGATCCGGGACCCCGGTCACCGTCTCCTCAGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACGCCAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGTGCCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTCTCGAGCGGTGGAGGCGGTTCAGGCGGAGGTGGAAGTGGTG
GTGGCGGCTCTGGAGGCGGCGGATCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCTGCGGGACCAAGGTGGAGATCAAAGGTGGTGGCGGCTCTGGAGGAGGAGGGT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGGCAGGCACCTGGG
AAGTGCCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTCAGGCCAGTCAGAACATTAGGACTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CTTGGAACCTGGCGATGCTGCAACTTACTATTGTCAGTCTACCTATCTTGGTACTGATTATGTT
GGCGGTGCTTTCGGCTGTGGGACCAAGGTGGAGATCAAATGA
>Sequence ID 19: SI-49P1 light chain moiety amino acid sequence
DIELTQSPAIMSASPGEKVTMTCSASSSVSYMHWYQQKSGTSPKRWIYDTSKLASGVPGRFSGS
GSGNSYSLTISSVEAEDDATYYCQQWSKHPLTFGSGTKVEIKRTVAAPSVFIFPPSDEQLKSGT
ASVVCLLNNFYPREAKVQWKVDNALQSGNSQESVTEQDSKDSTYSLSSTLTLSKADYEKHKVYA
CEVTHQGLSSPVTKSFNRGECGGGGSGGGGSFLNSLFNQEVQIPLTESYCGPCPKNWICYKNNC
YQFFDESKNWYESQASCMSQNASLLKVYSKEDQDLLKLVKSYHWMGLVHIPTNGSWQWEDGSIL
SPNLLTIIEMQKGDCALYASSFKGYIENCSTPNTYICMQRTVGGGGSGGGGSGGGGSGGGGSFL
NSLFNQEVQIPLTESYCGPCPKNWICYKNNCYQFFDESKNWYESQASCMSQNASLLKVYSKEDQ
DLLKLVKSYHWMGLVHIPTNGSWQWEDGSILSPNLLTIIEMQKGDCALYASSFKGYIENCSTPN
TYICMQRTV
>Sequence ID 20: SI-49P1 light chain moiety nucleotide sequence
GACATCGAGCTCACTCAGTCTCCAGCAATCATGTCTGCATCTCCAGGGGAGAAGGTCACCATGA
CCTGCAGTGCCAGCTCAAGTGTAAGTTACATGCACTGGTACCAGCAGAAGTCAGGCACCTCCCC
CAAAAGATGGATTTATGACACATCCAAACTGGCTTCTGGAGTCCCAGGTCGCTTCAGTGGCAGT
GGGTCTGGAAACTCTTACTCTCTCACAATCAGCAGCGTGGAGGCTGAAGATGATGCAACTTATT
ACTGCCAGCAGTGGAGTAAGCACCCTCTCACGTTCGGATCCGGGACCAAGGTGGAAATCAAACG
TACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGG
ATAACGCCCTCCAATCGGGTAACTCCCAGGAGAGTGTCACAGAGCAGGACAGCAAGGACAGCAC
CTACAGCCTCAGCAGCACCCTGACGCTGAGCAAAGCAGACTACGAGAAACACAAAGTCTACGCC
TGCGAAGTCACCCATCAGGGCCTGAGCTCGCCCGTCACAAAGAGCTTCAACAGGGGAGAGTGTG
GCGGTGGAGGGTCCGGCGGTGGTGGATCCTTTCTTAATTCCCTTTTCAACCAAGAGGTTCAGAT
CCCCTTGACTGAAAGCTATTGCGGCCCTTGTCCGAAAAACTGGATATGTTACAAGAATAATTGT
TAGCAATTCTTCGAGGAAAGCAAGAACTGGTATGAGAGTGAGGCGTCTTGTATGAGTCAGAATG
CCAGCCTGCTTAAGGTTTATTCAAAAGAAGACCAGGATCTGCTTAAGTTGGTAAAGAGCTACCA
CTGGATGGGGCTGGTACATATCCCAACGAATGGGTCATGGCAGTGGGAGGACGGTTCTATTCTG
AGTCCAAATCTCCTGACGATCATCGAAATGCAGAAAGGGGACTGTGCCCTGTATGCATCATCCT
TCAAGGGGTACATCGAGAACTGCAGTACCCCAAATACCTACATTTGTATGCAAAGAACGGTTGG
AGGCGGTGGCTCAGGCGGAGGCGGCTCAGGAGGTGGCGGTTCAGGAGGCGGCGGATCTTTCCTA
AACTCATTATTCAACCAAGAAGTTCAAATTCCCTTGACCGAAAGTTACTGTGGCCCATGTCCTA
AAAACTGGATATGTTACAAAAATAACTGCTACCAATTTTTTGATGAGAGTAAAAACTGGTATGA
GAGCCAGGCTTCTTGTATGTCTCAAAATGCCAGCCTTCTGAAAGTATACAGCAAAGAGGACCAG
GATTTAGTTAAACTGGTGAAGTCATATCATTGGATGGGACTAGTACACATTCCAACAAATGGAT
CTTGGCAGTGGGAAGATGGCTCCATTCTCTCACCCAACCTACTAACAATAATTGAAATGCAGAA
GGGAGACTGTGCACTCTATGCCTCGAGCTTTAAAGGCTATATAGAAAACTGTTCAACTCCAAAT
ACGTAGATCTGCATGCAAAGGACTGTGTAG
>Sequence ID 21: SI-49P3 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVLGGGGSGGGGSGGGGSG
GGGSEVQLVESGGGLVQPGGSLRLSCTASGFTISTNAMSWVRQAPGKCLEWVGVITGRDITYYA
SWAKGRFTISRDTSKNTVYLQMNSLRAEDTAVYYCARDGGSSAITSNNIWGQGTLVTVSTGGGG
SGGGGSEVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGTLVTVSSAS
TKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPEAAGGPSVFLFPPKP
KDTLMISRTPEVTCVVVDVSHEDPEVKFNWYVDGVEVHNAKTKPREEQYASTYRVVSVLTVLHQ
DWLNGKEYKCAVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQK
SLSLSPGGGGGSGGGGSEVQLLESGGGLVQPGGSLRLSCAASGFSFSSGYDMCWVRQAPGKCLE
WIACIAAGSAGITYDANWAKGRFTISRDNSKNTLYLQMNSLRAEDTAVYYCARSAFSFDYAMDL
WGQGTLVTVSSGGGGSGGGGSGGGGSGGGGSDIQMTQSPSTLSASVGDRVTITCQASQSISSHL
NWYQQKPGKAPKLLIYKASTLASGVPSRFSGSGSGTEFTLTISSLQPDDFATYYCQQGYSWGNV
DNVFGCGTKVEIKGGGGSGGGGSGRSLVESGGGLVQPGGSLRLSCTASGFTISSYHMQWVRQAP
GKCLEYIGTISSGGNVYYASSARGRFTISRPSSKNTVDLQMNSLRAEDTAVYYCARDSGYSDPM
WGQGTLVTVSSGGGGSGGGGSGGGGSGGGGSDVVMTQSPSSVSASVGDRVTITCQASQNIRTYL
SWYQQKPGKAPKLLIYAAANLASGVPSRFSGSGSGTDFTLTISDLEPGDAATYYCQSTYLGTDY
VGGAFGCGTKVEIK
>Sequence ID 22: SI-49P3 heavy chain nucleotide sequence
GAGATCGTGATGACCCAGAGCCCCAGCACCCTGAGCGCCAGCGTGGGCGACAGGGTGATCATCA
CCTGCCAGGCCAGCGAGAGCATCAGCAGCTGGCTGGCCTGGTACCAGCAGAAGCCCGGCAAGGC
CCCCAAGCTGCTGATCTACGAGGCCAGCAAGCTGGCCAGCGGCGTGCCCAGCAGGTTCAGCGGC
AGCGGCAGCGGCGCCGAGTTCACCCTGACCATCAGCAGCCTGCAGCCCGACGACTTCGCCACCT
ACTACTGCCAGGGCTACTTCTACTTCATCAGCAGGACCTACGTGAACAGCTTCGGCTGCGGCAC
CAAGCTGACCGTGCTGGGCGGTGGCGGTAGTGGGGGAGGCGGTTCTGGCGGCGGAGGGTCCGGC
GGTGGAGGATCAGAGGTGCAGCTGGTGGAGAGCGGCGGCGGCCTGGTGCAGCCCGGCGGCAGCC
TGAGGCTGAGCTGCACCGCCAGCGGCTTCACCATCAGCACCAACGCCATGAGCTGGGTGAGGCA
GGCCCCCGGCAAGTGCCTGGAGTGGGTGGGCGTGATCACCGGCAGGGACATCACCTACTACGCC
AGCTGGGCCAAGGGCAGGTTCACCATCAGCAGGGACACCAGCAAGAACACCGTGTACCTGCAGA
TGAACAGCCTGAGGGCCGAGGACACCGCCGTGTACTACTGCGCCAGGGACGGCGGCAGCAGCGC
CATCACCAGCAACAACATCTGGGGCCAGGGCACCCTGGTGACCGTGTCGACAGGCGGTGGAGGG
TCCGGCGGTGGTGGATCAGAGGTTCAGCTGGTGGAGTCTGGCGGTGGCCTGGTGCAGCCAGGGG
GCTCACTCCGTTTGTCCTGTGCAGCTTCTGGCTTCAACATTAAAGACACCTATATACACTGGGT
GCGTCAGGCCCCGGGTAAGGGCCTGGAATGGGTTGCAAGGATTTATCCTACGAATGGTTATACT
AGATATGCCGATAGCGTCAAGGGCCGTTTCACTATAAGCGCAGACACATCCAAAAACACAGCCT
ACCTGCAGATGAACAGCCTGCGTGCTGAGGACACTGCCGTCTATTATTGTTCTAGATGGGGAGG
GGACGGCTTCTATGCTATGGACTACTGGGGTCAAGGAACCCTGGTCACCGTCTCCTCGGCTAGC
ACCAAGGGCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAACTCAGGCGC
CCTGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCACA
AGCCCAGCAACACCAAGGTGGACAAGAGAGTTGAGCCCAAATCTTGTGACAAAACTCACACATG
CCCACCGTGCCCAGCACCTGAAGCCGCGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACG
AAGACCCTGAGGTCAAGTTCAACTGGTACGTGGACGGCGTGGAGGTGCATAATGCCAAGACAAA
GCCGCGGGAGGAGCAGTACGCCAGCACGTACCGTGTGGTCAGCGTCCTCACCGTCCTGCACCAG
GACTGGCTGAATGGCAAGGAGTACAAGTGCGCGGTCTCCAACAAAGCCCTCCCAGCCCCCATCG
AGAAAACCATCTCCAAAGCCAAAGGGCAGCCCCGAGAACCACAGGTGTACACCCTGCCCCCATC
CCGGGATGAGCTGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGACCACGCCTCCCG
TGCTGGACTCCGACGGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACCACTACACGCAGAAG
AGCCTCTCCCTGTCTCCGGGTGGCGGTGGAGGGTCCGGCGGTGGTGGATCCGAGGTGCAGCTGT
TGGAGTCTGGGGGAGGCTTGGTACAGCCTGGGGGGTCCCTGAGACTCTCCTGTGCAGCCTCTGG
ATTCTCCTTCAGTAGCGGGTACGACATGTGCTGGGTCCGCCAGGCTCCAGGGAAGTGCCTGGAG
TGGATCGCATGCATTGCTGCTGGTAGTGCTGGTATCACTTACGACGCGAACTGGGCGAAAGGCC
GGTTCACCATCTCCAGAGACAATTCCAAGAACACGCTGTATCTGCAAATGAACAGCCTGAGAGC
CGAGGACACGGCCGTATATTACTGTGCGAGATCGGCGTTTTCGTTCGACTACGCCATGGACCTC
TGGGGCCAGGGAACCCTGGTCACCGTCTCGAGCGGTGGAGGCGGTTCAGGCGGAGGTGGAAGTG
GTGGTGGCGGCTCTGGAGGCGGCGGATCTGACATCCAGATGACCCAGTCTCCTTCCACCCTGTC
TGCATCTGTAGGAGACAGAGTCACCATCACTTGCCAGGCCAGTCAGAGCATTAGTTCCCACTTA
AACTGGTATCAGCAGAAACCAGGGAAAGCCCCTAAGCTCCTGATCTATAAGGCATCCACTCTGG
CATCTGGGGTCCCATCAAGGTTCAGCGGCAGTGGATCTGGGACAGAATTTACTCTCACCATCAG
CAGCCTGCAGCCTGATGATTTTGCAACTTATTACTGCCAACAGGGTTATAGTTGGGGTAATGTT
GATAATGTTTTCGGCTGCGGGACCAAGGTGGAGATCAAAGGTGGTGGCGGCTCTGGAGGAGGAG
GGTCCGGACGGTCGCTGGTGGAGTCTGGGGGAGGCTTGGTCCAGCCTGGGGGGTCCCTGAGACT
CTCCTGTACTGCCTCTGGATTCACCATCAGTAGCTACCACATGCAGTGGGTCCGGCAGGCACCT
GGGAAGTGCCTGGAGTACATCGGAACCATTAGTAGTGGTGGTAATGTATACTACGCAAGCTCCG
CTAGAGGCAGATTCACCATCTCCAGACCCTCGTCCAAGAACACGGTGGATCTTCAAATGAACAG
CCTGAGAGCCGAGGACACGGCTGTGTATTACTGTGCGAGAGACTCTGGTTATAGTGATCCTATG
TGGGGCCAGGGAACCCTGGTCACCGTCTCTTCAGGCGGTGGCGGTAGTGGGGGAGGCGGTTCTG
GCGGCGGAGGGTCCGGCGGTGGAGGATCAGACGTTGTGATGACCCAGTCTCCATCTTCCGTGTC
TGCATCTGTAGGAGACAGAGTCACCATCACCTGTCAGGCCAGTCAGAACATTAGGACTTACTTA
TCCTGGTATCAGCAGAAACCAGGGAAAGCCCCTAAGCTCCTGATCTATGCTGCAGCCAATCTGG
CATCTGGGGTCCCATCAAGGTTCAGCGGCAGTGGATCTGGGACAGATTTCACTCTCACCATCAG
CGACTTGGAACCTGGCGATGCTGCAACTTACTATTGTCAGTCTACCTATCTTGGTACTGATTAT
GTTGGCGGTGCTTTCGGCTGTGGGACCAAGGTGGAGATCAAATGA
>Sequence ID 23: SI-49P3 light chain moiety amino acid sequence
DIQMTQSPSSLSASVGDRVTITCRASQDVNTAVAWYQQKPGKAPKLLIYSASFLYSGVPSRFSG
SRSGTDFTLTISSLQPEDFATYYCQQHYTTPPTFGQGTKVEIKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGECGGGGSGGGGSFLNSLFNQEVQIPLTESYCGPCPKNWICYKNN
CYQFFDESKNWYESQASCMSQNASLLKVYSKEDQDLLKLVKSYHWMGLVHIPTNGSWQWEDGSI
LSPNLLTIIEMQKGDCALYASSFKGYIENCSTPNTYICMQRTVGGGGSGGGGSGGGGSGGGGSF
LNSLFNQEVQIPLTESYCGPCPKNWICYKNNCYQFFDESKNWYESQASCMSQNASLLKVYSKED
QDLLKLVKSYHWMGLVHIPTNGSWQWEDGSILSPNLLTIIEMQKGDCALYASSFKGYIENCSTP
NTYICMQRTV
>Sequence ID 24: SI-49P3 light chain moiety nucleotide sequence
GATATCCAGATGACCCAGTCCCCGAGCTCCCTGTCCGCCTCTGTGGGCGATAGGGTCACCATCA
CCTGCCGTGCCAGTCAGGATGTGAATACTGCTGTAGCCTGGTATCAACAGAAACCAGGAAAAGC
TCCGAAACTACTGATTTACTCGGCATCCTTCCTCTACTCTGGAGTCCCTTCTCGCTTCTCTGGC
TCCAGATCTGGGACGGATTTCACTCTGACCATCAGCAGTCTGCAGCCGGAAGACTTCGCAACTT
ATTACTGTCAGCAACATTATACTACTCCTCCCACGTTCGGACAGGGTACCAAGGTGGAGATCAA
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTGGCGGTGGAGGGTCCGGCGGTGGTGGATCCTTTCTTAATTCCCTTTTCAACCAAGAGGTTCA
GATCCCCTTGACTGAAAGCTATTGCGGCCCTTGTCCGAAAAACTGGATATGTTACAAGAATAAT
TGTTACCAATTCTTCGAGGAAAGCAAGAACTGGTATGAGAGTGAGGCGTCTTGTATGAGTGAGA
ATGCCAGCCTGCTTAAGGTTTATTCAAAAGAAGACCAGGATCTGCTTAAGTTGGTAAAGAGCTA
CCACTGGATGGGGCTGGTACATATCCCAACGAATGGGTCATGGCAGTGGGAGGACGGTTCTATT
CTGAGTCCAAATCTCCTGACGATCATCGAAATGCAGAAAGGGGACTGTGCCCTGTATGCATCAT
CCTTCAAGGGGTACATCGAGAACTGCAGTACCCCAAATACCTACATTTGTATGCAAAGAACGGT
TGGAGGCGGTGGCTCAGGCGGAGGCGGCTCAGGAGGTGGCGGTTCAGGAGGCGGCGGATCTTTC
CTAAACTCATTATTCAACCAAGAAGTTCAAATTCCCTTGACCGAAAGTTACTGTGGCCCATGTC
CTAAAAACTGGATATGTTACAAAAATAACTGCTACCAATTTTTTGATGAGAGTAAAAACTGGTA
TGAGAGCCAGGCTTCTTGTATGTCTCAAAATGCCAGCCTTCTGAAAGTATACAGCAAAGAGGAC
CAGGATTTAGTTAAACTGGTGAAGTCATATCATTGGATGGGACTAGTACACATTCCAACAAATG
GATCTTGGCAGTGGGAAGATGGCTCCATTCTCTCACCCAACCTACTAACAATAATTGAAATGCA
GAAGGGAGACTGTGCACTCTATGCCTCGAGCTTTAAAGGCTATATAGAAAACTGTTCAACTCCA
AATACGTAGATCTGCATGCAAAGGACTGTGTAG
>Sequence ID 25: SI-55P3 heavy chain amino acid sequence
EIVMTQSPSTLSASVGDRVIITCRASQSIGTNIHWYQQKPGKAPKLLIYYASESISGIPSRFSG
SGSGAEFTLTISSLQPDDFATYYCQQNNNWPTTFGQGTKLTVLGGGGSGGGGSGGGGSGGGGSE
VQLVESGGGLVQPGGSLRLSCSVSGFSLTNYGVHWVRQAPGKGLEWVGVIWSGGNTDYNTPFTS
RFTISRDTSKNTVYLQMNSLRAEDTAVYYCARALTYYDYEFAYWGQGTLVTVSSGGGGSGGGGS
QVQLQESGGRLVQPGEPLSLTCKTSGIDLSSNAIGWVRQAPGKGLEWIGVIFGSGNTYYASWAK
GRFTISRSTSTVYLKMNSLRSEDTAIYYCARGGYSSDIWGQGTLVTVSSASTKGPSVFPLAPSS
KSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVT
CVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGGGGGSG
GGGSEVQLLESGGGLVQPGGSLRLSCAASGFSFSSGYDMCWVRQAPGKGLEWIACIAAGSAGIT
YDANWAKGRFTISRDNSKNTLYLQMNSLRAEDTAVYYCARSAFSFDYAMDLWGQGTLVTVSSGG
GGSGGGGSGGGGSGGGGSDIQMTQSPSTLSASVGDRVTITCQASQSISSHLNWYQQKPGKAPKL
LIYKASTLASGVPSRFSGSGSGTEFTLTISSLQPDDFATYYCQQGYSWGNVDNVFGGGTKVEIK
GGGGSGGGGSGRSLVESGGGLVQPGGSLRLSCTASGFTISSYHMQWVRQAPGKGLEYIGTISSG
GNVYYASSARGRFTISRPSSKNTVDLQMNSLRAEDTAVYYCARDSGYSDPMWGQGTLVTVSSGG
GGSGGGGSGGGGSGGGGSDVVMTQSPSSVSASVGDRVTITCQASQNIRTYLSWYQQKPGKAPKL
LIYAAANLASGVPSRFSGSGSGTDFTLTISDLEPGDAATYYCQSTYLGTDYVGGAFGGGTKVEI
K
>Sequence ID 26: SI-55P3 heavy chain nucleotide sequence
GAAATCGTTATGACACAGTCCCCATCCACTCTTAGCGCTTCTGTAGGGGATCGAGTGATTATCA
CATGCCGGGCCTCCCAATCCATAGGAACCAACATACACTGGTATCAACAAAAACCAGGCAAAGC
GCCAAAACTGCTTATCTACTACGCCTCCGAGAGTATTTCTGGAATCCCGAGTCGCTTCTCAGGT
TCTGGAAGCGGCGCTGAGTTTACCCTCACAATTTCTTCACTCCAACCGGATGACTTCGCTACAT
ATTACTGCCAACAAAACAATAATTGGCCGACGACCTTTGGCCAGGGCACGAAACTTACGGTACT
TGGCGGTGGCGGTAGTGGGGGAGGCGGTTCTGGCGGCGGAGGGTCCGGCGGTGGAGGATCAGAA
GTACAGCTTGTCGAGTCCGGTGGGGGGCTTGTTCAGCCAGGGGGTTCCTTGAGGCTTTCCTGCT
CCGTCTCTGGGTTTAGCTTGACGAATTACGGCGTTCACTGGGTTAGACAAGCACCGGGGAAGGG
GCTGGAATGGGTCGGTGTGATATGGTCCGGGGGTAATACGGATTACAATACACCTTTCACGTCA
CGCTTTACGATTAGCAGGGACACGTCAAAAAATACAGTCTACTTGCAGATGAACTCTCTTAGGG
CGGAAGATACTGCAGTTTATTACTGCGCAAGGGCTCTGACATACTACGATTATGAATTTGCATA
TTGGGGCCAGGGGACTTTGGTCACGGTCTCGAGCGGCGGTGGAGGGTCCGGCGGTGGTGGATCA
CAAGTGCAGTTGCAAGAAAGTGGTGGTAGACTGGTTCAGCCTGGTGAACCCTTGTCACTGACGT
GTAAAACAAGCGGCATTGATCTGTCCTCTAACGCCATCGGATGGGTCCGACAGGCCCCAGGAAA
AGGTCTGGAGTGGATCGGAGTTATCTTCGGGAGCGGCAATACATACTACGCAAGCTGGGCAAAA
GGGCGATTTACGATATCACGGAGCACCTCTACAGTTTATTTGAAAATGAACTCCCTCCGGTCCG
AGGATACCGCGATATATTACTGTGCCAGAGGGGGGTACTCCTCTGATATCTGGGGGCAGGGTAC
ACTGGTTACAGTTTCATCCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCA
AATCTTGTGACAAAACTCACACATGCCCACCGTGCCCAGCACCTGAAGCCGCGGGGGCACCGTC
AGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGCGGTGGAGGGTCCGGC
GGTGGTGGATCCGAGGTGCAGCTGTTGGAGTCTGGGGGAGGCTTGGTACAGCCTGGGGGGTCCC
TGAGACTCTCCTGTGCAGCCTCTGGATTCTCCTTCAGTAGCGGGTACGACATGTGCTGGGTCCG
CCAGGCTCCAGGGAAGGGGCTGGAGTGGATCGCATGCATTGCTGCTGGTAGTGCTGGTATCACT
TACGACGCGAACTGGGCGAAAGGCCGGTTCACCATCTCCAGAGACAATTCCAAGAACACGCTGT
ATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTGTCGAGCGGTGGA
GGCGGATCTGGCGGAGGTGGTTCCGGCGGTGGCGGCTCCGGTGGAGGCGGCTCTGACATCCAGA
TGACCCAGTCTCCTTCCACCCTGTCTGCATCTGTAGGAGACAGAGTCACCATCACTTGCCAGGC
CAGTCAGAGCATTAGTTCCCACTTAAACTGGTATCAGCAGAAACCAGGGAAAGCCCCTAAGCTC
CTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCGGAGGGACCAAGGTGGAGATCAAA
GGCGGTGGAGGGTCCGGCGGTGGTGGCTCCGGACGGTCGCTGGTGGAGTCTGGGGGAGGCTTGG
TCCAGCCTGGGGGGTCCCTGAGACTCTCCTGTACTGCCTCTGGATTCACCATCAGTAGCTACCA
CATGCAGTGGGTCCGCCAGGCTCCAGGGAAGGGGCTGGAGTACATCGGAACCATTAGTAGTGGT
GGTAATGTATACTACGCAAGCTCCGCTAGAGGCAGATTCACCATCTCCAGACCCTCGTCCAAGA
ACACGGTGGATCTTCAAATGAACAGCCTGAGAGCCGAGGACACGGCTGTGTATTACTGTGCGAG
AGACTCTGGTTATAGTGATCCTATGTGGGGCCAGGGAACCCTGGTCACCGTCTCTTCAGGCGGT
GGCGGTAGTGGGGGAGGCGGTTCTGGCGGCGGAGGGTCCGGCGGTGGAGGATCAGACGTTGTGA
TGACCCAGTCTCCATCTTCCGTGTCTGCATCTGTAGGAGACAGAGTCACCATCACCTGTCAGGC
CAGTCAGAACATTAGGACTTACTTATCCTGGTATCAGCAGAAACCAGGGAAAGCCCCTAAGCTC
CTGATCTATGCTGCAGCCAATCTGGCATCTGGGGTCCCATCAAGGTTCAGCGGCAGTGGATCTG
GGACAGATTTCACTCTCACCATCAGCGACCTGGAGCCTGGCGATGCTGCAACTTACTATTGTCA
GTCTACCTATCTTGGTACTGATTATGTTGGCGGTGCTTTCGGCGGAGGGACCAAGGTGGAGATC
AAATGA
>Sequence ID 27: SI-55P3 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGEC
>Sequence ID 28: SI-55P3 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGTGGCGGTGGAGGGTCCGGCGGTGGTGGATCC
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAGCGTACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACA
GGGGAGAGTGTTGA
>Sequence ID 29: SI-55P4 heavy chain amino acid sequence
EIVMTQSPSTLSASVGDRVIITCRASQSIGTNIHWYQQKPGKAPKLLIYYASESISGIPSRFSG
SGSGAEFTLTISSLQPDDFATYYCQQNNNWPTTFGQGTKLTVLGGGGSGGGGSGGGGSGGGGSE
VQLVESGGGLVQPGGSLRLSCSVSGFSLTNYGVHWVRQAPGKGLEWVGVIWSGGNTDYNTPFTS
RFTISRDTSKNTVYLQMNSLRAEDTAVYYCARALTYYDYEFAYWGQGTLVTVSSGGGGSGGGGS
QVQLQESGGRLVQPGEPLSLTCKTSGIDLSSNAIGWVRQAPGKGLEWIGVIFGSGNTYYASWAK
GRFTISRSTSTVYLKMNSLRSEDTAIYYCARGGYSSDIWGQGTLVTVSSASTKGPSVFPLAPSS
KSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVT
CVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGGGGGSG
GGGSEVQLLESGGGLVQPGGSLRLSCAASGFSFSSGYDMCWVRQAPGKGLEWIACIAAGSAGIT
YDANWAKGRFTISRDNSKNTLYLQMNSLRAEDTAVYYCARSAFSFDYAMDLWGQGTLVTVSSGG
GGSGGGGSGGGGSGGGGSDIQMTQSPSTLSASVGDRVTITCQASQSISSHLNWYQQKPGKAPKL
LIYKASTLASGVPSRFSGSGSGTEFTLTISSLQPDDFATYYCQQGYSWGNVDNVFGGGTKVEIK
GGGGSGGGGSGREGPELSPDDPAGLLDLRQGMFAQLVAQNVLLIDGPLSWYSDPGLAGVSLTGG
LSYKEDTKELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPEIPAGLGST
GSGSKPGSGEGSTKGREGPELSPDDPAGLLDLRQGMFAQLVAQNVLLIDGPLSWYSDPGLAGVS
LTGGLSYKEDTKELVVAKAGVYYVFFQLELRRVVAGEGSGSVSLALHLQPLRSAAGAAALALTV
DLPPASSEARNSAFGFQGRLLHLSAGQRLGVHLHTEARARHAWQLTQGATVLGLFRVTPEIPAG
LGGGGSGGGGSREGPELSPDDPAGLLDLRQGMFAQLVAQNVLLIDGPLSWYSDPGLAGVSLTGG
LSYKEDTKELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPEIPAGL
>Sequence ID 30: SI-55P4 heavy chain nucleotide sequence
GAAATCGTTATGACACAGTCCCCATCCACTCTTAGCGCTTCTGTAGGGGATCGAGTGATTATCA
CATGCCGGGCCTCCCAATCCATAGGAACCAACATACACTGGTATCAACAAAAACCAGGCAAAGC
GCCAAAACTGCTTATCTACTACGCCTCCGAGAGTATTTCTGGAATCCCGAGTCGCTTCTCAGGT
TCTGGAAGCGGCGCTGAGTTTACCCTCACAATTTCTTCACTCCAACCGGATGACTTCGCTACAT
ATTACTGCCAACAAAACAATAATTGGCCGACGACCTTTGGCCAGGGCACGAAACTTACGGTACT
TGGCGGTGGCGGTAGTGGGGGAGGCGGTTCTGGCGGCGGAGGGTCCGGCGGTGGAGGATCAGAA
GTACAGCTTGTCGAGTCCGGTGGGGGGCTTGTTCAGCCAGGGGGTTCCTTGAGGCTTTCCTGCT
CCGTCTCTGGGTTTAGCTTGACGAATTACGGCGTTCACTGGGTTAGACAAGCACCGGGGAAGGG
GCTGGAATGGGTCGGTGTGATATGGTCCGGGGGTAATACGGATTACAATACACCTTTCACGTCA
CGCTTTACGATTAGCAGGGACACGTCAAAAAATACAGTCTACTTGCAGATGAACTCTCTTAGGG
CGGAAGATACTGCAGTTTATTACTGCGCAAGGGCTCTGACATACTACGATTATGAATTTGCATA
TTGGGGCCAGGGGACTTTGGTCACGGTCTCGAGCGGCGGTGGAGGGTCCGGCGGTGGTGGATCA
CAAGTGCAGTTGCAAGAAAGTGGTGGTAGACTGGTTCAGCCTGGTGAACCCTTGTCACTGACGT
GTAAAACAAGCGGCATTGATCTGTCCTCTAACGCCATCGGATGGGTCCGACAGGCCCCAGGAAA
AGGTCTGGAGTGGATCGGAGTTATCTTCGGGAGCGGCAATACATACTACGCAAGCTGGGCAAAA
GGGCGATTTACGATATCACGGAGCACCTCTACAGTTTATTTGAAAATGAACTCCCTCCGGTCCG
AGGATACCGCGATATATTACTGTGCCAGAGGGGGGTACTCCTCTGATATCTGGGGGCAGGGTAC
ACTGGTTACAGTTTCATCCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCA
AATCTTGTGACAAAACTCACACATGCCCACCGTGCCCAGCACCTGAAGCCGCGGGGGCACCGTC
AGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGCGGTGGAGGGTCCGGC
GGTGGTGGATCCGAGGTGCAGCTGTTGGAGTCTGGGGGAGGCTTGGTACAGCCTGGGGGGTCCC
TGAGACTCTCCTGTGCAGCCTCTGGATTCTCCTTCAGTAGCGGGTACGACATGTGCTGGGTCCG
CCAGGCTCCAGGGAAGGGGCTGGAGTGGATCGCATGCATTGCTGCTGGTAGTGCTGGTATCACT
TACGACGCGAACTGGGCGAAAGGCCGGTTCACCATCTCCAGAGACAATTCCAAGAACACGCTGT
ATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTGTCGAGCGGTGGA
GGCGGATCTGGCGGAGGTGGTTCCGGCGGTGGCGGCTCCGGTGGAGGCGGCTCTGACATCCAGA
TGACCCAGTCTCCTTCCACCCTGTCTGCATCTGTAGGAGACAGAGTCACCATCACTTGCCAGGC
CAGTCAGAGCATTAGTTCCCACTTAAACTGGTATCAGCAGAAACCAGGGAAAGCCCCTAAGCTC
CTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCGGAGGGACCAAGGTGGAGATCAAA
GGCGGTGGAGGGTCCGGCGGTGGTGGCTCCGGACGAGAGGGCCCCGAGCTGTCTCCTGATGACC
CAGCAGGCCTCTTGGACTTGCGGCAGGGTATGTTCGCTCAACTTGTGGCTCAGAATGTTCTGCT
CATTGATGGACCACTCTCTTGGTATAGTGACCCCGGTCTGGCCGGGGTGAGTCTGACCGGCGGG
CTCTCTTATAAAGAGGATACTAAGGAACTGGTCGTAGCAAAAGCGGGCGTTTATTACGTTTTTT
TTCAGCTGGAGCTCAGGCGCGTGGTGGCCGGCGAGGGCAGTGGCTCTGTGTCCCTGGCCCTGCA
CTTACAGCCCTTGAGAAGCGCTGCAGGTGCTGCCGCACTGGCTTTAACTGTTGACCTCCCTCCG
GCCTCTTCTGAAGCTAGAAACAGCGCTTTCGGCTTCCAAGGGCGCCTGCTGCACCTGAGCGCAG
GCCAGCGCTTAGGTGTGCACCTTCATACAGAGGCCAGGGCCCGACACGCTTGGCAGCTCACACA
GGGTGCCACGGTTCTCGGACTTTTCCGCGTTACTCCCGAGATCCCCGCTGGCCTCGGAAGTACT
GGTTCTGGGTCTAAACCCGGTTCCGGCGAAGGTAGTACTAAAGGACGAGAAGGGCCAGAGTTAA
GTCCAGATGACCCTGCTGGGCTTTTGGACCTGCGGCAGGGCATGTTCGCTCAACTGGTGGCTCA
GAACGTGCTGCTGATCGATGGCCCCCTGAGTTGGTACAGCGATCCCGGGCTGGCAGGCGTGTCA
CTTACAGGGGGCCTCTCTTACAAGGAAGACACCAAGGAGTTAGTGGTCGCTAAGGCTGGCGTGT
ATTACGTGTTCTTCCAACTGGAGCTGAGAAGGGTTGTGGCAGGAGAGGGTAGCGGCAGCGTGTC
TTTAGCCCTTCACTTGCAGCCCCTGAGGTCTGCTGCAGGTGCAGCCGCTCTCGCGCTCACCGTG
GATCTCCCCCCAGCCTCATCTGAAGCTAGGAACAGTGCATTTGGCTTTCAGGGACGCTTGCTGC
ACCTCTCCGCTGGACAGAGGCTGGGCGTGCACCTTCACACAGAGGCCCGTGCCAGGCATGCATG
GCAGCTCACTCAGGGGGCAACAGTGCTGGGTCTCTTCCGCGTGACTCCTGAAATACCAGCTGGA
CTTGGCGGTGGAGGCAGCGGCGGAGGAGGATCTCGTGAGGGGCCAGAACTGTCCCCCGATGACC
CAGCCGGACTGCTCGATCTCAGACAGGGCATGTTCGCTCAGCTTGTAGCCCAAAATGTCCTCCT
GATTGACGGCCCTTTGAGCTGGTATAGTGATCCCGGCTTGGCCGGGGTATCTCTGACCGGAGGC
CTCTCCTACAAGGAAGACACCAAAGAGCTGGTGGTGGCAAAAGCGGGGGTGTATTATGTGTTCT
TTCAGCTCGAGCTGCGGAGAGTTGTGGCCGGGGAAGGGTCTGGGAGCGTATCTCTTGCACTTCA
CCTGCAGCCCCTGCGCAGCGCCGCTGGAGCCGCCGCCCTTGCTCTTACTGTGGATCTGCCTCCT
GCTTCCTCAGAAGCACGCAACAGCGCCTTCGGCTTTCAAGGACGTCTCCTGCACTTGTCCGCAG
GACAGAGGTTGGGCGTCCATTTACACACTGAGGCACGGGCACGGCACGCTTGGCAGCTTACCCA
GGGAGCCACCGTGCTGGGACTCTTTAGAGTGACACCCGAGATCCCCGCTGGCTTGTGA
>Sequence ID 31: SI-55P4 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGEC
>Sequence ID 32: SI-55P4 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGTGGCGGTGGAGGGTCCGGCGGTGGTGGATCC
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAGCGTACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACA
GGGGAGAGTGTTGA
>Sequence ID 33: SI-55P9 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
EVQLVESGGGLVQPGGSLRLSCAASGFTISTNAMSWVRQAPGKGLEWIGVITGRDITYYASWAK
GRFTISRDNSKNTLYLQMNSLRAEDTAVYYCARDGGSSAITSNNIWGQGTLVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKRVEPKSCDKTHTCPPCPAPEAAGAPSVFLFPPKPKDTLMI
SRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSP
GGGGGSGGGGSEVQLLESGGGLVQPGGSLRLSCAASGFSFSSGYDMCWVRQAPGKGLEWIACIA
AGSAGITYDANWAKGRFTISRDNSKNTLYLQMNSLRAEDTAVYYCARSAFSFDYAMDLWGQGTL
VTVSSGGGGSGGGGSGGGGSGGGGSDIQMTQSPSTLSASVGDRVTITCQASQSISSHLNWYQQK
PGKAPKLLIYKASTLASGVPSRFSGSGSGTEFTLTISSLQPDDFATYYCQQGYSWGNVDNVFGG
GTKVEIKGGGGSGGGGSGRSLVESGGGLVQPGGSLRLSCTASGFTISSYHMQWVRQAPGKGLEY
IGTISSGGNVYYASSARGRFTISRPSSKNTVDLQMNSLRAEDTAVYYCARDSGYSDPMWGQGTL
VTVSSGGGGSGGGGSGGGGSGGGGSDVVMTQSPSSVSASVGDRVTITCQASQNIRTYLSWYQQK
PGKAPKLLIYAAANLASGVPSRFSGSGSGTDFTLTISDLEPGDAATYYCQSTYLGTDYVGGAFG
GGTKVEIK
>Sequence ID 34: SI-55P9 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGCTGACCGTTTT
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGCGGTGGAGGGTCCGGCGGTGGTGGATCA
GAGGTGCAGCTGGTGGAGTCTGGGGGAGGCTTGGTCCAGCCTGGGGGGTCCCTGAGACTCTCCT
GTGCAGCCTCTGGATTCACCATCAGTACCAATGCAATGAGCTGGGTCCGCCAGGCTCCAGGGAA
GGGGCTGGAGTGGATCGGAGTCATTACTGGTCGTGATATCACATACTACGCGAGCTGGGCGAAA
GGCAGATTCACCATCTCCAGAGACAATTCCAAGAACACGCTGTATCTTCAAATGAACAGCCTGA
GAGCCGAGGACACGGCTGTGTATTACTGTGCGAGAGACGGTGGTTCTTCTGCTATTACTAGTAA
CAACATTTGGGGCCAGGGAACCCTGGTCACCGTGTCCTCAGCTAGCACCAAGGGCCCATCGGTC
TTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGG
TGGACAAGAGAGTTGAGCCCAAATCTTGTGACAAAACTCACACATGCCCACCGTGCCCAGCACC
TGAAGCCGCGGGGGCACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAG
GAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCG
GGTGGCGGTGGAGGGTCCGGCGGTGGTGGATCCGAGGTGCAGCTGTTGGAGTCTGGGGGAGGCT
TGGTACAGCCTGGGGGGTCCCTGAGACTCTCCTGTGCAGCCTCTGGATTCTCCTTCAGTAGCGG
GTACGACATGTGCTGGGTCCGCCAGGCTCCAGGGAAGGGGCTGGAGTGGATCGCATGCATTGCT
GCTGGTAGTGCTGGTATCACTTACGACGCGAACTGGGCGAAAGGCCGGTTCACCATCTCCAGAG
ACAATTCCAAGAACACGCTGTATCTGCAAATGAACAGCCTGAGAGCCGAGGACACGGCCGTATA
TTACTGTGCGAGATCGGCGTTTTCGTTCGACTACGCCATGGACCTCTGGGGCCAGGGAACCCTG
GTCACCGTGTCGAGCGGTGGAGGCGGATCTGGCGGAGGTGGTTCCGGCGGTGGCGGCTCCGGTG
GAGGCGGCTCTGACATCCAGATGACCCAGTCTCCTTCCACCCTGTCTGCATCTGTAGGAGACAG
AGTCACCATCACTTGCCAGGCCAGTCAGAGCATTAGTTCCCACTTAAACTGGTATCAGCAGAAA
CCAGGGAAAGCCCCTAAGCTCCTGATCTATAAGGCATCCACTCTGGCATCTGGGGTCCCATCAA
GGTTCAGCGGCAGTGGATCTGGGACAGAATTTACTCTCACCATCAGCAGCCTGCAGCCTGATGA
TTTTGCAACTTATTACTGCCAACAGGGTTATAGTTGGGGTAATGTTGATAATGTTTTCGGCGGA
GGGACCAAGGTGGAGATCAAAGGCGGTGGAGGGTCCGGCGGTGGTGGCTCCGGACGGTCGCTGG
TGGAGTCTGGGGGAGGCTTGGTCCAGCCTGGGGGGTCCCTGAGACTCTCCTGTACTGCCTCTGG
ATTCACCATCAGTAGCTACCACATGCAGTGGGTCCGCCAGGCTCCAGGGAAGGGGCTGGAGTAC
ATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACAC
GGCTGTGTATTACTGTGCGAGAGACTCTGGTTATAGTGATCCTATGTGGGGCCAGGGAACCCTG
GTCACCGTCTCTTCAGGCGGTGGCGGTAGTGGGGGAGGCGGTTCTGGCGGCGGAGGGTCCGGCG
GTGGAGGATCAGACGTTGTGATGACCCAGTCTCCATCTTCCGTGTCTGCATCTGTAGGAGACAG
AGTCACCATCACCTGTCAGGCCAGTCAGAACATTAGGACTTACTTATCCTGGTATCAGCAGAAA
CCAGGGAAAGCCCCTAAGCTCCTGATCTATGCTGCAGCCAATCTGGCATCTGGGGTCCCATCAA
GGTTCAGCGGCAGTGGATCTGGGACAGATTTCACTCTCACCATCAGCGACCTGGAGCCTGGCGA
TGCTGCAACTTACTATTGTCAGTCTACCTATCTTGGTACTGATTATGTTGGCGGTGCTTTCGGC
GGAGGGACCAAGGTGGAGATCAAATGA
>Sequence ID 35: SI-55P9 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGGGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC
>Sequence ID 36: SI-55P9 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGCGGCGGTGGAGGGTCCGGCGGTGGTGGATCA
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTCACTCTCACCATCAGCAGCCTGCAGCCTGATGATTTTGCAACTT
ATTACTGCCAAGGCTATTTTTATTTTATTAGTCGTACTTATGTAAATTCTTTCGGCGGAGGGAC
CAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTTGA
>Sequence ID 37: SI-55P10 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
EVQLVESGGGLVQPGGSLRLSCAASGFTISTNAMSWVRQAPGKGLEWIGVITGRDITYYASWAK
GRFTISRDNSKNTLYLQMNSLRAEDTAVYYCARDGGSSAITSNNIWGQGTLVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKRVEPKSCDKTHTCPPCPAPEAAGAPSVFLFPPKPKDTLMI
SRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSP
GGGGGSGGGGSEVQLLESGGGLVQPGGSLRLSCAASGFSFSSGYDMCWVRQAPGKGLEWIACIA
AGSAGITYDANWAKGRFTISRDNSKNTLYLQMNSLRAEDTAVYYCARSAFSFDYAMDLWGQGTL
VTVSSGGGGSGGGGSGGGGSGGGGSDIQMTQSPSTLSASVGDRVTITCQASQSISSHLNWYQQK
PGKAPKLLIYKASTLASGVPSRFSGSGSGTEFTLTISSLQPDDFATYYCQQGYSWGNVDNVFGG
GTKVEIKGGGGSGGGGSGREGPELSPDDPAGLLDLRQGMFAQLVAQNVLLIDGPLSWYSDPGLA
GVSLTGGLSYKEDTKELVVAKAGVYYVFFQLELRRVVAGEGSGSVSLALHLQPLRSAAGAAALA
LTVDLPPASSEARNSAFGFQGRLLHLSAGQRLGVHLHTEARARHAWQLTQGATVLGLFRVTPEI
PAGLGSTGSGSKPGSGEGSTKGREGPELSPDDPAGLLDLRQGMFAQLVAQNVLLIDGPLSWYSD
PGLAGVSLTGGLSYKEDTKELVVAKAGVYYVFFQLELRRVVAGEGSGSVSLALHLQPLRSAAGA
AALALTVDLPPASSEARNSAFGFQGRLLHLSAGQRLGVHLHTEARARHAWQLTQGATVLGLFRV
TPEIPAGLGGGGSGGGGSREGPELSPDDPAGLLDLRQGMFAQLVAQNVLLIDGPLSWYSDPGLA
GVSLTGGLSYKEDTKELVVAKAGVYYVFFQLELRRVVAGEGSGSVSLALHLQPLRSAAGAAALA
LTVDLPPASSEARNSAFGFQGRLLHLSAGQRLGVHLHTEARARHAWQLTQGATVLGLFRVTPEI
PAGL
>Sequence ID 38: SI-55P10 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGCTGACCGTTTT
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGCGGTGGAGGGTCCGGCGGTGGTGGATCA
GAGGTGCAGCTGGTGGAGTCTGGGGGAGGCTTGGTCCAGCCTGGGGGGTCCCTGAGACTCTCCT
GTGCAGCCTCTGGATTCACCATCAGTACCAATGCAATGAGCTGGGTCCGCCAGGCTCCAGGGAA
GGGGCTGGAGTGGATCGGAGTCATTACTGGTCGTGATATCACATACTACGCGAGCTGGGCGAAA
GGCAGATTCACCATCTCCAGAGACAATTCCAAGAACACGCTGTATCTTCAAATGAACAGCCTGA
GAGCCGAGGACACGGCTGTGTATTACTGTGCGAGAGACGGTGGTTCTTCTGCTATTACTAGTAA
CAACATTTGGGGCCAGGGAACCCTGGTCACCGTGTCCTCAGCTAGCACCAAGGGCCCATCGGTC
TTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAAGG
TGGACAAGAGAGTTGAGCCCAAATCTTGTGACAAAACTCACACATGCCCACCGTGCCCAGCACC
TGAAGCCGCGGGGGCACCGTCAGTCTTCCTCTTCCCCCCAAAACCCAAGGACACCCTCATGATC
TCCCGGACCCCTGAGGTCACATGCGTGGTGGTGGACGTGAGCCACGAAGACCCTGAGGTCAAGT
TCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAG
GAGTACAAGTGCGCGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCG
GGTGGCGGTGGAGGGTCCGGCGGTGGTGGATCCGAGGTGCAGCTGTTGGAGTCTGGGGGAGGCT
TGGTACAGCCTGGGGGGTCCCTGAGACTCTCCTGTGCAGCCTCTGGATTCTCCTTCAGTAGCGG
GTACGACATGTGCTGGGTCCGCCAGGCTCCAGGGAAGGGGCTGGAGTGGATCGCATGCATTGCT
GCTGGTAGTGCTGGTATCACTTACGACGCGAACTGGGCGAAAGGCCGGTTCACCATCTCCAGAG
ACAATTCCAAGAACACGCTGTATCTGCAAATGAACAGCCTGAGAGCCGAGGACACGGCCGTATA
TTACTGTGCGAGATCGGCGTTTTCGTTCGACTACGCCATGGACCTCTGGGGCCAGGGAACCCTG
GTCACCGTGTCGAGCGGTGGAGGCGGATCTGGCGGAGGTGGTTCCGGCGGTGGCGGCTCCGGTG
GAGGCGGCTCTGACATCCAGATGACCCAGTCTCCTTCCACCCTGTCTGCATCTGTAGGAGACAG
AGTCACCATCACTTGCCAGGCCAGTCAGAGCATTAGTTCCCACTTAAACTGGTATCAGCAGAAA
CCAGGGAAAGCCCCTAAGCTCCTGATCTATAAGGCATCCACTCTGGCATCTGGGGTCCCATCAA
GGTTCAGCGGCAGTGGATCTGGGACAGAATTTACTCTCACCATCAGCAGCCTGCAGCCTGATGA
TTTTGCAACTTATTACTGCCAACAGGGTTATAGTTGGGGTAATGTTGATAATGTTTTCGGCGGA
GGGACCAAGGTGGAGATCAAAGGCGGTGGAGGGTCCGGCGGTGGTGGCTCCGGACGAGAGGGCC
CCGAGCTGTCTCCTGATGACCCAGCAGGCCTCTTGGACTTGCGGCAGGGTATGTTCGCTCAACT
TGTGGCTCAGAATGTTCTGCTCATTGATGGACCACTCTCTTGGTATAGTGACCCCGGTCTGGCC
GGGGTGAGTCTGACCGGCGGGCTCTCTTATAAAGAGGATACTAAGGAACTGGTCGTAGCAAAAG
CGGGCGTTTATTACGTTTTTTTTCAGCTGGAGCTCAGGCGCGTGGTGGCCGGCGAGGGCAGTGG
CTCTGTGTCCCTGGCCCTGCACTTACAGCCCTTGAGAAGCGCTGCAGGTGCTGCCGCACTGGCT
TTAACTGTTGACCTCCCTCCGGCCTCTTCTGAAGCTAGAAACAGCGCTTTCGGCTTCCAAGGGC
GCCTGCTGCACCTGAGCGCAGGCCAGCGCTTAGGTGTGCACCTTCATACAGAGGCCAGGGCCCG
ACACGCTTGGCAGCTCACACAGGGTGCCACGGTTCTCGGACTTTTCCGCGTTACTCCCGAGATC
CCCGCTGGCCTCGGAAGTACTGGTTCTGGGTCTAAACCCGGTTCCGGCGAAGGTAGTACTAAAG
GACGAGAAGGGCCAGAGTTAAGTCCAGATGACCCTGCTGGGCTTTTGGACCTGCGGCAGGGCAT
GTTCGCTCAACTGGTGGCTCAGAACGTGCTGCTGATCGATGGCCCCCTGAGTTGGTACAGCGAT
CCCGGGCTGGCAGGCGTGTCACTTACAGGGGGCCTCTCTTACAAGGAAGACACCAAGGAGTTAG
TGGTCGCTAAGGCTGGCGTGTATTACGTGTTCTTCCAACTGGAGCTGAGAAGGGTTGTGGCAGG
AGAGGGTAGCGGCAGCGTGTCTTTAGCCCTTCACTTGCAGCCCCTGAGGTCTGCTGCAGGTGCA
GCCGCTCTCGCGCTCACCGTGGATCTCCCCCCAGCCTCATCTGAAGCTAGGAACAGTGCATTTG
GCTTTCAGGGACGCTTGCTGCACCTCTCCGCTGGACAGAGGCTGGGCGTGCACCTTCACACAGA
GGCCCGTGCCAGGCATGCATGGCAGCTCACTCAGGGGGCAACAGTGCTGGGTCTCTTCCGCGTG
ACTCCTGAAATACCAGCTGGACTTGGCGGTGGAGGCAGCGGCGGAGGAGGATCTCGTGAGGGGC
CAGAACTGTCCCCCGATGACCCAGCCGGACTGCTCGATCTCAGACAGGGCATGTTCGCTCAGCT
TGTAGCCCAAAATGTCCTCCTGATTGACGGCCCTTTGAGCTGGTATAGTGATCCCGGCTTGGCC
GGGGTATCTCTGACCGGAGGCCTCTCCTACAAGGAAGACACCAAAGAGCTGGTGGTGGCAAAAG
CGGGGGTGTATTATGTGTTCTTTCAGCTCGAGCTGCGGAGAGTTGTGGCCGGGGAAGGGTCTGG
GAGCGTATCTCTTGCACTTCACCTGCAGCCCCTGCGCAGCGCCGCTGGAGCCGCCGCCCTTGCT
CTTACTGTGGATCTGCCTCCTGCTTCCTCAGAAGCACGCAACAGCGCCTTCGGCTTTCAAGGAC
GTCTCCTGCACTTGTCCGCAGGACAGAGGTTGGGCGTCCATTTACACACTGAGGCACGGGCACG
GCACGCTTGGCAGCTTACCCAGGGAGCCACCGTGCTGGGACTCTTTAGAGTGACACCCGAGATC
CCCGCTGGCTTGTGA
>Sequence ID 39: SI-55P10 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGGGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC
>Sequence ID 40: SI-55P10 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGCGGCGGTGGAGGGTCCGGCGGTGGTGGATCA
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTCACTCTCACCATCAGCAGCCTGCAGCCTGATGATTTTGCAACTT
ATTACTGCCAAGGCTATTTTTATTTTATTAGTCGTACTTATGTAAATTCTTTCGGCGGAGGGAC
CAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTTGA
>Sequence ID 41: SI-77P1 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGQGTKLTVLGGGGSGGGGSGGGGSG
GGGSEVQLVESGGGLVQPGGSLRLSCAASGFTISTNAMSWVRQAPGKGLEWVGVITGRDITYYA
SWAKGRFTISRDTSKNTVYLQMNSLRAEDTAVYYCARDGGSSAITSNNIWGQGTLVTVSSGGGG
SGGGGSQVQLQQSGPGLVKPSETLSITCTVSGFSLTNYGVHWIRQAPGKGLEWLGVIWSGGNTD
YNTPFTSRFTITKDNSKNQVYFKLRSVRADDTAIYYCARALTYYDYEFAYWGQGTLVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGAPSVFLFPPKPK
DTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQKS
LSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGGGTKVEIKGGGGSGGGGSGRSLVESGGGLVQPGGSLRLSCTASGFTISSYHMQWVRQAPG
KGLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGGGTKVEIK
>Sequence ID 42: SI-77P1 heavy chain nucleotide sequence
GAAATCGTTATGACGCAGAGTCCCTCCACGCTCTCCGCTAGTGTCGGGGATCGCGTCATTATCA
CATGCCAGGCCTCCGAGTCAATCAGCAGCTGGCTTGCATGGTATCAACAGAAGCCGGGAAAAGC
TCCTAAATTGCTGATCTATGAAGCGTCAAAATTGGCGTCTGGTGTCCCATCTAGGTTCTCCGGC
TCTGGGTCTGGTGCGGAATTTACTTTGACAATCTCCAGTCTTCAACCAGACGATTTCGCTACCT
ACTACTGCCAAGGGTATTTCTATTTTATAAGCCGGACATATGTAAACTCCTTCGGCCAAGGAAC
AAAGTTGACTGTTCTTGGTGGCGGAGGCAGTGGTGGCGGGGGCAGCGGAGGTGGTGGTTCAGGG
GGTGGTGGGAGCGAAGTCCAATTGGTAGAAAGTGGCGGTGGTCTGGTGCAACCTGGTGGATCTC
TTCGCCTCTCATGCGCCGCTAGTGGCTTTACTATTTCAACTAATGCGATGAGCTGGGTTCGCCA
GGCCCCCGGCAAAGGACTTGAGTGGGTCGGCGTCATCACCGGCAGGGACATTACATACTATGCG
AGTTGGGCAAAGGGCAGGTTCACGATTAGCCGCGATACTTCAAAGAATACCGTTTACCTTCAAA
TGAATAGCTTGAGGGCGGAAGACACAGCTGTGTATTACTGCGCGAGGGATGGAGGTAGTTCCGC
CATAACTTCCAACAACATATGGGGACAAGGCACGCTGGTTACTGTCTCGAGTGGCGGTGGAGGG
TCCGGCGGTGGTGGATCACAAGTACAGTTGCAGCAATCCGGTCCCGGTCTCGTCAAACCGAGTG
AGACGCTTAGTATAACGTGTACTGTTTCAGGCTTTAGCCTTACGAACTATGGAGTTCACTGGAT
TCGGCAGGCACCCGGCAAAGGTTTGGAATGGCTGGGTGTTATTTGGTCAGGTGGAAATACAGAC
TATAACACCCCCTTTACAAGTCGGTTCACAATTAGGAAAGATAATTCCAAAAATCAAGTTTATT
TCAAGTTGAGATCCGTCCGCGCGGACGACACTGCGATCTACTATTGTGCGAGGGCACTGACCTA
CTACGATTACGAATTTGCGTATTGGGGGCAAGGGACTCTTGTAACAGTCTCCAGTGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTGTCGAGCGGTGGAGGCGGATCTGGCGGAGGTGGTTCCGGCG
GTGGCGGCTCCGGTGGAGGCGGCTCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCGGAGGGACCAAGGTGGAGATCAAAGGCGGTGGAGGGTCCGGCGGTGGTGGCT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGCCAGGCTCCAGGG
AAGGGGCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTCAGGCCAGTCAGAACATTAGGACTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAA
>Sequence ID 43: SI-77P1 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLSLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGEC
>Sequence ID 44: SI-77P1 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTAGG
CGGTGGCGGTAGTGGGGGAGGCGGTTCTGGCGGCGGAGGGTCCGGCGGTGGAGGATCACAGGTC
ACATTGAAGGAATCTGGCCCCGGCCTTGTTCAGCCAGGACAGACCCTTAGCCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGTGGCGGTGGAGGGTCCGGCGGTGGTGGATCA
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGCTGACCGTTTT
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTTAG
>Sequence ID 45: SI-79P2 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLELKGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
QVQLQESGGRLVQPGEPLSLTCKTSGIDLSSNAIGWVRQAPGKGLEWIGVIFGSGNTYYASWAK
GRFTISRSTSTVYLKMNSLRSEDTAIYYCARGGYSSDIWGQGTLVTVSSASTKGPSVFPLAPSS
KSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVT
CVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGGGGGSG
GGGSEVQLLESGGGLVQPGGSLRLSCAASGFSFSSGYDMCWVRQAPGKGLEWIACIAAGSAGIT
YDANWAKGRFTISRDNSKNTLYLQMNSLRAEDTAVYYCARSAFSFDYAMDLWGQGTLVTVSSGG
GGSGGGGSGGGGSGGGGSDIQMTQSPSTLSASVGDRVTITCQASQSISSHLNWYQQKPGKAPKL
LIYKASTLASGVPSRFSGSGSGTEFTLTISSLQPDDFATYYCQQGYSWGNVDNVFGGGTKVEIK
GGGGSGGGGSGRSLVESGGGLVQPGGSLRLSCTASGFTISSYHMQWVRQAPGKGLEYIGTISSG
GNVYYASSARGRFTISRPSSKNTVDLQMNSLRAEDTAVYYCARDSGYSDPMWGQGTLVTVSSGG
GGSGGGGSGGGGSGGGGSDVVMTQSPSSVSASVGDRVTITCQASQNIRTYLSWYQQKPGKAPKL
LIYAAANLASGVPSRFSGSGSGTDFTLTISDLEPGDAATYYCQSTYLGTDYVGGAFGGGTKVEI
K
>Sequence ID 46: SI-79P2 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGTTGGAACTGAA
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGCGGTGGAGGGTCCGGCGGTGGTGGATCA
CAAGTGCAGTTGCAAGAAAGTGGTGGTAGACTGGTTCAGCCTGGTGAACCCTTGTCACTGACGT
GTAAAACAAGCGGCATTGATCTGTCCTCTAACGCCATCGGATGGGTCCGACAGGCCCCAGGAAA
AGGTCTGGAGTGGATCGGAGTTATCTTCGGGAGCGGCAATACATACTACGCAAGCTGGGCAAAA
GGGCGATTTACGATATCACGGAGCACCTCTACAGTTTATTTGAAAATGAACTCCCTCCGGTCCG
AGGATACCGCGATATATTACTGTGCCAGAGGGGGGTACTCCTCTGATATCTGGGGGCAGGGTAC
ACTGGTTACAGTTTCATCCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCA
AATCTTGTGACAAAACTCACACATGCCCACCGTGCCCAGCACCTGAAGCCGCGGGGGCACCGTC
AGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGCGGTGGAGGGTCCGGC
GGTGGTGGATCCGAGGTGCAGCTGTTGGAGTCTGGGGGAGGCTTGGTACAGCCTGGGGGGTCCC
TGAGACTCTCCTGTGCAGCCTCTGGATTCTCCTTCAGTAGCGGGTACGACATGTGCTGGGTCCG
CCAGGCTCCAGGGAAGGGGCTGGAGTGGATCGCATGCATTGCTGCTGGTAGTGCTGGTATCACT
TACGACGCGAACTGGGCGAAAGGCCGGTTCACCATCTCCAGAGACAATTCCAAGAACACGCTGT
ATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTGTCGAGCGGTGGA
GGCGGATCTGGCGGAGGTGGTTCCGGCGGTGGCGGCTCCGGTGGAGGCGGCTCTGACATCCAGA
TGACCCAGTCTCCTTCCACCCTGTCTGCATCTGTAGGAGACAGAGTCACCATCACTTGCCAGGC
CAGTCAGAGCATTAGTTCCCACTTAAACTGGTATCAGCAGAAACCAGGGAAAGCCCCTAAGCTC
CTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCGGAGGGACCAAGGTGGAGATCAAA
GGCGGTGGAGGGTCCGGCGGTGGTGGCTCCGGACGGTCGCTGGTGGAGTCTGGGGGAGGCTTGG
TCCAGCCTGGGGGGTCCCTGAGACTCTCCTGTACTGCCTCTGGATTCACCATCAGTAGCTACCA
CATGCAGTGGGTCCGCCAGGCTCCAGGGAAGGGGCTGGAGTACATCGGAACCATTAGTAGTGGT
GGTAATGTATACTACGCAAGCTCCGCTAGAGGCAGATTCACCATCTCCAGACCCTCGTCCAAGA
ACACGGTGGATCTTCAAATGAACAGCCTGAGAGCCGAGGACACGGCTGTGTATTACTGTGCGAG
AGACTCTGGTTATAGTGATCCTATGTGGGGCCAGGGAACCCTGGTCACCGTCTCTTCAGGCGGT
GGCGGTAGTGGGGGAGGCGGTTCTGGCGGCGGAGGGTCCGGCGGTGGAGGATCAGACGTTGTGA
TGACCCAGTCTCCATCTTCCGTGTCTGCATCTGTAGGAGACAGAGTCACCATCACCTGTCAGGC
CAGTCAGAACATTAGGACTTACTTATCCTGGTATCAGCAGAAACCAGGGAAAGCCCCTAAGCTC
CTGATCTATGCTGCAGCCAATCTGGCATCTGGGGTCCCATCAAGGTTCAGCGGCAGTGGATCTG
GGACAGATTTCACTCTCACCATCAGCGACCTGGAGCCTGGCGATGCTGCAACTTACTATTGTCA
GTCTACCTATCTTGGTACTGATTATGTTGGCGGTGCTTTCGGCGGAGGGACCAAGGTGGAGATC
AAATGA
>Sequence ID 47: SI-79P2 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGEC
>Sequence ID 48: SI-79P2 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGTGGCGGTGGAGGGTCCGGCGGTGGTGGATCC
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAGCGTACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACA
GGGGAGAGTGTTGA
>Sequence ID 49: SI-79P3 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLELKGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
QVQLQESGGRLVQPGEPLSLTCKTSGIDLSSNAIGWVRQAPGKGLEWIGVIFGSGNTYYASWAK
GRFTISRSTSTVYLKMNSLRSEDTAIYYCARGGYSSDIWGQGTLVTVSSASTKGPSVFPLAPSS
KSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVT
CVVVDVSHEDPEVKFNWYVDGVEVHNAKTKPREEQYNSTYRVVSVLTVLHQDWLNGKEYKCAVS
NKALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPGGGGGSG
GGGSEVQLLESGGGLVQPGGSLRLSCAASGFSFSSGYDMCWVRQAPGKGLEWIACIAAGSAGIT
YDANWAKGRFTISRDNSKNTLYLQMNSLRAEDTAVYYCARSAFSFDYAMDLWGQGTLVTVSSGG
GGSGGGGSGGGGSGGGGSDIQMTQSPSTLSASVGDRVTITCQASQSISSHLNWYQQKPGKAPKL
LIYKASTLASGVPSRFSGSGSGTEFTLTISSLQPDDFATYYCQQGYSWGNVDNVFGGGTKVEIK
GGGGSGGGGSGREGPELSPDDPAGLLDLRQGMFAQLVAQNVLLIDGPLSWYSDPGLAGVSLTGG
LSYKEDTKELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPEIPAGLGST
GSGSKPGSGEGSTKGREGPELSPDDPAGLLDLRQGMFAQLVAQNVLLIDGPLSWYSDPGLAGVS
LTGGLSYKEDTKELVVAKAGVYYVFFQLELRRVVAGEGSGSVSLALHLQPLRSAAGAAALALTV
DLPPASSEARNSAFGFQGRLLHLSAGQRLGVHLHTEARARHAWQLTQGATVLGLFRVTPEIPAG
LGGGGSGGGGSREGPELSPDDPAGLLDLRQGMFAQLVAQNVLLIDGPLSWYSDPGLAGVSLTGG
LSYKEDTKELVVAKAGVYYVFFQLELRRVVAGEGSGSVSLALHLQPLRSAAGAAALALTVDLPP
ASSEARNSAFGFQGRLLHLSAGQRLGVHLHTEARARHAWQLTQGATVLGLFRVTPEIPAGL
>Sequence ID 50: SI-79P3 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGTTGGAACTGAA
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGCGGTGGAGGGTCCGGCGGTGGTGGATCA
CAAGTGCAGTTGCAAGAAAGTGGTGGTAGACTGGTTCAGCCTGGTGAACCCTTGTCACTGACGT
GTAAAACAAGCGGCATTGATCTGTCCTCTAACGCCATCGGATGGGTCCGACAGGCCCCAGGAAA
AGGTCTGGAGTGGATCGGAGTTATCTTCGGGAGCGGCAATACATACTACGCAAGCTGGGCAAAA
GGGCGATTTACGATATCACGGAGCACCTCTACAGTTTATTTGAAAATGAACTCCCTCCGGTCCG
AGGATACCGCGATATATTACTGTGCCAGAGGGGGGTACTCCTCTGATATCTGGGGGCAGGGTAC
ACTGGTTACAGTTTCATCCGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCA
AATCTTGTGACAAAACTCACACATGCCCACCGTGCCCAGCACCTGAAGCCGCGGGGGCACCGTC
AGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCG
TGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCC
AACAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGC
TCTGCACAACCACTACACGCAGAAGAGCCTCTCCCTGTCTCCGGGTGGCGGTGGAGGGTCCGGC
GGTGGTGGATCCGAGGTGCAGCTGTTGGAGTCTGGGGGAGGCTTGGTACAGCCTGGGGGGTCCC
TGAGACTCTCCTGTGCAGCCTCTGGATTCTCCTTCAGTAGCGGGTACGACATGTGCTGGGTCCG
CCAGGCTCCAGGGAAGGGGCTGGAGTGGATCGCATGCATTGCTGCTGGTAGTGCTGGTATCACT
TACGACGCGAACTGGGCGAAAGGCCGGTTCACCATCTCCAGAGACAATTCCAAGAACACGCTGT
ATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTGTCGAGCGGTGGA
GGCGGATCTGGCGGAGGTGGTTCCGGCGGTGGCGGCTCCGGTGGAGGCGGCTCTGACATCCAGA
TGACCCAGTCTCCTTCCACCCTGTCTGCATCTGTAGGAGACAGAGTCACCATCACTTGCCAGGC
CAGTCAGAGCATTAGTTCCCACTTAAACTGGTATCAGCAGAAACCAGGGAAAGCCCCTAAGCTC
CTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCGGAGGGACCAAGGTGGAGATCAAA
GGCGGTGGAGGGTCCGGCGGTGGTGGCTCCGGACGAGAGGGCCCCGAGCTGTCTCCTGATGACC
CAGCAGGCCTCTTGGACTTGCGGCAGGGTATGTTCGCTCAACTTGTGGCTCAGAATGTTCTGCT
CATTGATGGACCACTCTCTTGGTATAGTGACCCCGGTCTGGCCGGGGTGAGTCTGACCGGCGGG
CTCTCTTATAAAGAGGATACTAAGGAACTGGTCGTAGCAAAAGCGGGCGTTTATTACGTTTTTT
TTCAGCTGGAGCTCAGGCGCGTGGTGGCCGGCGAGGGCAGTGGCTCTGTGTCCCTGGCCCTGCA
CTTACAGCCCTTGAGAAGCGCTGCAGGTGCTGCCGCACTGGCTTTAACTGTTGACCTCCCTCCG
GCCTCTTCTGAAGCTAGAAACAGCGCTTTCGGCTTCCAAGGGCGCCTGCTGCACCTGAGCGCAG
GCCAGCGCTTAGGTGTGCACCTTCATACAGAGGCCAGGGCCCGACACGCTTGGCAGCTCACACA
GGGTGCCACGGTTCTCGGACTTTTCCGCGTTACTCCCGAGATCCCCGCTGGCCTCGGAAGTACT
GGTTCTGGGTCTAAACCCGGTTCCGGCGAAGGTAGTACTAAAGGACGAGAAGGGCCAGAGTTAA
GTCCAGATGACCCTGCTGGGCTTTTGGACCTGCGGCAGGGCATGTTCGCTCAACTGGTGGCTCA
GAACGTGCTGCTGATCGATGGCCCCCTGAGTTGGTACAGCGATCCCGGGCTGGCAGGCGTGTCA
CTTACAGGGGGCCTCTCTTACAAGGAAGACACCAAGGAGTTAGTGGTCGCTAAGGCTGGCGTGT
ATTACGTGTTCTTCCAACTGGAGCTGAGAAGGGTTGTGGCAGGAGAGGGTAGCGGCAGCGTGTC
TTTAGCCCTTCACTTGCAGCCCCTGAGGTCTGCTGCAGGTGCAGCCGCTCTCGCGCTCACCGTG
GATCTCCCCCCAGCCTCATCTGAAGCTAGGAACAGTGCATTTGGCTTTCAGGGACGCTTGCTGC
ACCTCTCCGCTGGACAGAGGCTGGGCGTGCACCTTCACACAGAGGCCCGTGCCAGGCATGCATG
GCAGCTCACTCAGGGGGCAACAGTGCTGGGTCTCTTCCGCGTGACTCCTGAAATACCAGCTGGA
CTTGGCGGTGGAGGCAGCGGCGGAGGAGGATCTCGTGAGGGGCCAGAACTGTCCCCCGATGACC
CAGCCGGACTGCTCGATCTCAGACAGGGCATGTTCGCTCAGCTTGTAGCCCAAAATGTCCTCCT
GATTGACGGCCCTTTGAGCTGGTATAGTGATCCCGGCTTGGCCGGGGTATCTCTGACCGGAGGC
CTCTCCTACAAGGAAGACACCAAAGAGCTGGTGGTGGCAAAAGCGGGGGTGTATTATGTGTTCT
TTCAGCTCGAGCTGCGGAGAGTTGTGGCCGGGGAAGGGTCTGGGAGCGTATCTCTTGCACTTCA
CCTGCAGCCCCTGCGCAGCGCCGCTGGAGCCGCCGCCCTTGCTCTTACTGTGGATCTGCCTCCT
GCTTCCTCAGAAGCACGCAACAGCGCCTTCGGCTTTCAAGGACGTCTCCTGCACTTGTCCGCAG
GACAGAGGTTGGGCGTCCATTTACACACTGAGGCACGGGCACGGCACGCTTGGCAGCTTACCCA
GGGAGCCACCGTGCTGGGACTCTTTAGAGTGACACCCGAGATCCCCGCTGGCTTGTGA
>Sequence ID 51: SI-79P3 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLRLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGEC
>Sequence ID 52: SI-79P3 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACGGTACTGGG
TGGAGGCGGTTCAGGCGGAGGTGGTTCCGGCGGTGGCGGCTCCGGTGGAGGCGGCTCTCAGGTC
ACATTGAAGGAATCTGGCCCCGGCCTTGTTCAGCCAGGACAGACCCTTAGGCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCGCGGATGGAACTCTGGTCTTACTACTTTGATTA
TTGGGGGCAGGGGACTCTCGTCACGGTCTCGAGTGGCGGTGGAGGGTCCGGCGGTGGTGGATCC
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAGCGTACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTCACAGAGCAGGACAG
CAAGGACAGCACCTACAGCCTCAGCAGCACCCTGACGCTGAGCAAAGCAGACTACGAGAAACAC
AAAGTCTACGCCTGCGAAGTCACCCATCAGGGCCTGAGCTCGCCCGTCACAAAGAGCTTCAACA
GGGGAGAGTGTTGA
>Sequence ID 53: SI-55H11 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
GGGGSGGGGSEVQLVESGGGLVQPGGSLRLSCAASGFTISTNAMSWVREAPGKCLEWIGVITGR
DITYYASWAKGRFTISRDNSKNTLYLQMNSLRAEDTAVYYCARDGGSSAITSNNIWGQGTLVTV
SSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLT
VLHQDWLNGKEYKCAVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNH
YTQKSLSLSPGGGGGSGGGGSEVQLLESGGGLVQPGGSLRLSCAASGFSFSSGYDMCWVRQAPG
KGLEWIACIAAGSAGITYDANWAKGRFTISRDNSKNTLYLQMNSLRAEDTAVYYCARSAFSFDY
AMDLWGQGTLVTVSSGGGGSGGGGSGGGGSGGGGSDIQMTQSPSTLSASVGDRVTITCQASQSI
SSHLNWYQQKPGKAPKLLIYKASTLASGVPSRFSGSGSGTEFTLTISSLQPDDFATYYCQQGYS
WGNVDNVFGGGTKVEIKGGGGSGGGGSGRSLVESGGGLVQPGGSLRLSCTASGFTISSYHMQWV
RQAPGKGLEYIGTISSGGNVYYASSARGRFTISRPSSKNTVDLQMNSLRAEDTAVYYCARDSGY
SDPMWGQGTLVTVSSGGGGSGGGGSGGGGSGGGGSDVVMTQSPSSVSASVGDRVTITCQASQNI
RTYLSWYQQKPGKAPKLLIYAAANLASGVPSRFSGSGSGTDFTLTISDLEPGDAATYYCQSTYL
GTDYVGGAFGGGTKVEIK
>Sequence ID 54: SI-55H11 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGCTGACCGTTTT
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGTGGAGGCGGATCTGGCGGAGGTGGTTCC
GGCGGTGGCGGCTCCGGTGGAGGCGGCTCTGAGGTGCAGCTGGTGGAGTCTGGGGGAGGCTTGG
TCCAGCCTGGGGGGTCCCTGAGACTCTCCTGTGCAGCCTCTGGATTCACCATCAGTACCAATGC
AATGAGCTGGGTCCGCGAGGCTCCAGGGAAGTGTCTGGAGTGGATCGGAGTCATTACTGGTCGT
GATATCACATACTACGCGAGCTGGGCGAAAGGCAGATTCACCATCTCCAGAGACAATTCCAAGA
ACACGCTGTATCTTCAAATGAACAGCCTGAGAGCCGAGGACACGGCTGTGTATTACTGTGCGAG
AGACGGTGGTTCTTCTGCTATTACTAGTAACAACATTTGGGGCCAGGGAACCCTGGTCACCGTG
TCCTCAGCTAGCACCAAGGGCCCATCGGTCTTCCCCCTGGCACCCTCCTCCAAGAGCACCTCTG
GGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTC
CCCCCAAAACCCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACAC
CCTGCCCCCATCCCGGGATGAGCTGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTATAGCAAGCTCACCGTGGACAA
GAGCAGGTGGCAGCAGGGGAACGTCTTCTCATGCTCCGTGATGCATGAGGCTCTGCACAACCAC
TACACGCAGAAGAGCCTCTCCCTGTCTCCGGGTGGCGGTGGAGGGTCCGGCGGTGGTGGATCCG
AGGTGCAGCTGTTGGAGTCTGGGGGAGGCTTGGTACAGCCTGGGGGGTCCCTGAGACTCTCCTG
TGCAGCCTCTGGATTCTCCTTCAGTAGCGGGTACGACATGTGCTGGGTCCGCCAGGCTCCAGGG
AAGGGGCTGGAGTGGATCGCATGCATTGCTGCTGGTAGTGCTGGTATCACTTACGACGCGAACT
GGGCGAAAGGCCGGTTCACCATCTCCAGAGACAATTCCAAGAACACGCTGTATCTGCAAATGAA
CAGCCTGAGAGCCGAGGACACGGCCGTATATTACTGTGCGAGATCGGCGTTTTCGTTCGACTAC
GCCATGGACCTCTGGGGCCAGGGAACCCTGGTCACCGTGTCGAGCGGTGGAGGCGGATCTGGCG
GAGGTGGTTCCGGCGGTGGCGGCTCCGGTGGAGGCGGCTCTGACATCCAGATGACCCAGTCTCC
TTCCACCCTGTCTGCATCTGTAGGAGACAGAGTCACCATCACTTGCCAGGCCAGTCAGAGCATT
AGTTCCCACTTAAACTGGTATCAGCAGAAACCAGGGAAAGCCCCTAAGCTCCTGATCTATAAGG
CATCCACTCTGGCATCTGGGGTCCCATCAAGGTTCAGCGGCAGTGGATCTGGGACAGAATTTAC
TCTCACCATCAGCAGCCTGCAGCCTGATGATTTTGCAACTTATTACTGCCAACAGGGTTATAGT
TGGGGTAATGTTGATAATGTTTTCGGCGGAGGGACCAAGGTGGAGATCAAAGGCGGTGGAGGGT
CCGGCGGTGGTGGCTCCGGACGGTCGCTGGTGGAGTCTGGGGGAGGCTTGGTCCAGCCTGGGGG
GTCCCTGAGACTCTCCTGTACTGCCTCTGGATTCACCATCAGTAGCTACCACATGCAGTGGGTC
CGCCAGGCTCCAGGGAAGGGGCTGGAGTACATCGGAACCATTAGTAGTGGTGGTAATGTATACT
ACGCAAGCTCCGCTAGAGGCAGATTCACCATCTCCAGACCCTCGTCCAAGAACACGGTGGATCT
TCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCTTCAGGCGGTGGCGGTAGTGGGG
GAGGCGGTTCTGGCGGCGGAGGGTCCGGCGGTGGAGGATCAGACGTTGTGATGACCCAGTCTCC
ATCTTCCGTGTCTGCATCTGTAGGAGACAGAGTCACCATCACCTGTCAGGCCAGTCAGAACATT
AGGACTTACTTATCCTGGTATCAGCAGAAACCAGGGAAAGCCCCTAAGCTCCTGATCTATGCTG
CAGCCAATCTGGCATCTGGGGTCCCATCAAGGTTCAGCGGCAGTGGATCTGGGACAGATTTCAC
TCTCACCATCAGCGACCTGGAGCCTGGCGATGCTGCAACTTACTATTGTCAGTCTACCTATCTT
GGTACTGATTATGTTGGCGGTGCTTTCGGCGGAGGGACCAAGGTGGAGATCAAATGA
>Sequence ID 55: SI-55H11 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLSLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGCGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGECGGGGSGGGGSGGGGSQSALTQPASVSGSPGQSITISC
TGTSSDVGGYNFVSWYQQHPGKAPKLMIYDVSDRPSGVSDRFSGSKSGNTASLIISGLQADDEA
DYYCSSYGSSSTHVIFGGGTKVTVLGGGGSGGGGSGGGGSGGGGSQVQLQESGGGLVKPGGSLS
LSCAASGFTFSSYWMSWVRQAPGKGLEWVANINRDGSASYYVDSVKGRFTISRDDAKNSLYLQM
NSLRAEDTAVYYCARDRGVGYFDLWGRGTLVTVSS
>Sequence ID 56: SI-55H11 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTAGG
CGGTGGCGGTAGTGGGGGAGGCGGTTCTGGCGGCGGAGGGTCCGGCGGTGGAGGATCACAGGTC
ACATTGAAGGAATCTGGCCCCGGCCTTGTTCAGCCAGGACAGACCCTTAGCCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCTCGGATGGAACTCTGGTCTTACTACTTTGATTA
TTGGGGGCAGGGGACTCTCGTCACGGTCTCGAGTGGCGGTGGAGGGTCCGGCGGTGGTGGATCA
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAAGGCTATTTTTATTTTATTAGTCGTACTTATGTAAATTCTTTCGGCTGTGGGAC
CAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTGGCGGTGGCGGTAGCGGTGGCGGCGGAAGTGGTGGCGGAGGATCCCA
GTCTGCCCTGACTCAGCCTGCCTCCGTGTCTGGGTCTCCTGGACAGTCGATCACCATCTCCTGC
ACTGGAACCAGCAGTGACGTTGGTGGTTATAACTTTGTCTCCTGGTACCAACAACACCCAGGCA
AAGCCCCCAAACTCATGATCTATGATGTCAGTGATCGGCCCTCAGGGGTGTCTGATCGCTTCTC
CGGCTCCAAGTCTGGCAACACGGCCTCCCTGATCATCTCTGGCCTCCAGGCTGACGACGAGGCT
GATTATTACTGCAGCTCATATGGGAGCAGCAGCACTCATGTGATTTTCGGCGGAGGGACCAAGG
TGACCGTCCTAGGTGGAGGCGGTTCAGGCGGAGGTGGTTCCGGCGGTGGCGGCTCCGGTGGAGG
CGGCTCTCAGGTGCAATTGCAGGAGTCGGGGGGAGGCCTGGTCAAGCCTGGAGGGTCCCTGAGT
CTCTCCTGTGCAGCCTCTGGATTCACCTTTAGTAGTTATTGGATGAGCTGGGTCCGCCAGGCTC
CAGGGAAGGGGCTGGAGTGGGTGGCCAACATAAACCGCGATGGAAGTGCGAGTTACTATGTGGA
CTCTGTGAAGGGCCGATTCACCATCTCCAGAGACGACGCCAAGAACTCACTGTATCTGCAAATG
AACAGCCTGAGAGCTGAGGACACGGCTGTGTATTACTGTGCGAGAGATCGTGGGGTGGGCTACT
TCGATCTCTGGGGCCGTGGCACCCTGGTCACCGTCTCTAGCTGA
>Sequence ID 57: SI-55H12 heavy chain amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVLGGGGSGGGGSGGGGSGGGGSQ
VQLQQSGPGLVKPSETLSITCTVSGFSLTNYGVHWIRQAPGKGLEWLGVIWSGGNTDYNTPFTS
RFTITKDNSKNQVYFKLRSVRADDTAIYYCARALTYYDYEFAYWGQGTLVTVSSGGGGSGGGGS
GGGGSGGGGSEVQLVESGGGLVQPGGSLRLSCAASGFTISTNAMSWVREAPGKGLEWIGVITGR
DITYYASWAKGRFTISRDNSKNTLYLQMNSLRAEDTAVYYCARDGGSSAITSNNIWGQGTLVTV
SSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPEAAGAPSVFLF
PPKPKDTLMISRTPEVTCVVVDVSHEDPEVKFNWYVDGVEVHNAKTKPREEQYNSTYRVVSVLT
VLHQDWLNGKEYKCAVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNH
YTQKSLSLSPGGGGGSGGGGSEVQLLESGGGLVQPGGSLRLSCAASGFSFSSGYDMCWVRQAPG
KGLEWIACIAAGSAGITYDANWAKGRFTISRDNSKNTLYLQMNSLRAEDTAVYYCARSAFSFDY
AMDLWGQGTLVTVSSGGGGSGGGGSGGGGSGGGGSDIQMTQSPSTLSASVGDRVTITCQASQSI
SSHLNWYQQKPGKAPKLLIYKASTLASGVPSRFSGSGSGTEFTLTISSLQPDDFATYYCQQGYS
WGNVDNVFGGGTKVEIKGGGGSGGGGSGRSLVESGGGLVQPGGSLRLSCTASGFTISSYHMQWV
RQAPGKGLEYIGTISSGGNVYYASSARGRFTISRPSSKNTVDLQMNSLRAEDTAVYYCARDSGY
SDPMWGQGTLVTVSSGGGGSGGGGSGGGGSGGGGSDVVMTQSPSSVSASVGDRVTITCQASQNI
RTYLSWYQQKPGKAPKLLIYAAANLASGVPSRFSGSGSGTDFTLTISDLEPGDAATYYCQSTYL
GTDYVGGAFGGGTKVEIK
>Sequence ID 58: SI-55H12 heavy chain nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGACCCGGTACAAAGCTGACCGTTTT
AGGCGGTGGCGGTAGTGGGGGAGGCGGTTCTGGCGGCGGAGGGTCCGGCGGTGGAGGATCACAA
GTACAGTTGCAGCAATCCGGTCCCGGTCTCGTCAAACCGAGTGAGACGCTTAGTATAACGTGTA
CTGTTTCAGGCTTTAGCCTTACGAACTATGGAGTTCACTGGATTCGGCAGGCACCCGGCAAAGG
TTTGGAATGGCTGGGTGTTATTTGGTCAGGTGGAAATACAGACTATAACACCCCCTTTACAAGT
CGGTTCACAATTACGAAAGATAATTCCAAAAATCAAGTTTATTTCAAGTTGAGATCCGTCCGCG
CGGACGACACTGCGATCTACTATTGTGCGAGGGCACTGACCTACTACGATTACGAATTTGCGTA
TTGGGGGCAAGGGACTCTTGTAACAGTCTCGAGCGGTGGAGGCGGATCTGGCGGAGGTGGTTCC
GGCGGTGGCGGCTCCGGTGGAGGCGGCTCTGAGGTGCAGCTGGTGGAGTCTGGGGGAGGCTTGG
TCCAGCCTGGGGGGTCCCTGAGACTCTCCTGTGCAGCCTCTGGATTCACCATCAGTACCAATGC
AATGAGCTGGGTCCGCGAGGCTCCAGGGAAGGGCCTGGAGTGGATCGGAGTCATTACTGGTCGT
GATATCACATACTACGCGAGCTGGGCGAAAGGCAGATTCACCATCTCCAGAGACAATTCCAAGA
ACACGCTGTATCTTCAAATGAACAGCCTGAGAGCCGAGGACACGGCTGTGTATTACTGTGCGAG
AGACGGTGGTTCTTCTGCTATTACTAGTAACAACATTTGGGGCCAGGGAACCCTGGTCACCGTG
TCCTCAGCTAGCACCAAGGGCCCATCGGTCTTCCCCCTGGCACCCTCCTCCAAGAGCACCTCTG
GGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTC
CCCCCAAAACCCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACAC
CCTGCCCCCATCCCGGGATGAGCTGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTATAGCAAGCTCACCGTGGACAA
GAGCAGGTGGCAGCAGGGGAACGTCTTCTCATGCTCCGTGATGCATGAGGCTCTGCACAACCAC
TACACGCAGAAGAGCCTCTCCCTGTCTCCGGGTGGCGGTGGAGGGTCCGGCGGTGGTGGATCCG
AGGTGCAGCTGTTGGAGTCTGGGGGAGGCTTGGTACAGCCTGGGGGGTCCCTGAGACTCTCCTG
TGCAGCCTCTGGATTCTCCTTCAGTAGCGGGTACGACATGTGCTGGGTCCGCCAGGCTCCAGGG
AAGGGGCTGGAGTGGATCGCATGCATTGCTGCTGGTAGTGCTGGTATCACTTACGACGCGAACT
GGGCGAAAGGCCGGTTCACCATCTCCAGAGACAATTCCAAGAACACGCTGTATCTGCAAATGAA
CAGCCTGAGAGCCGAGGACACGGCCGTATATTACTGTGCGAGATCGGCGTTTTCGTTCGACTAC
GCCATGGACCTCTGGGGCCAGGGAACCCTGGTCACCGTGTCGAGCGGTGGAGGCGGATCTGGCG
GAGGTGGTTCCGGCGGTGGCGGCTCCGGTGGAGGCGGCTCTGACATCCAGATGACCCAGTCTCC
TTCCACCCTGTCTGCATCTGTAGGAGACAGAGTCACCATCACTTGCCAGGCCAGTCAGAGCATT
AGTTCCCACTTAAACTGGTATCAGCAGAAACCAGGGAAAGCCCCTAAGCTCCTGATCTATAAGG
CATCCACTCTGGCATCTGGGGTCCCATCAAGGTTCAGCGGCAGTGGATCTGGGACAGAATTTAC
TCTCACCATCAGCAGCCTGCAGCCTGATGATTTTGCAACTTATTACTGCCAACAGGGTTATAGT
TGGGGTAATGTTGATAATGTTTTCGGCGGAGGGACCAAGGTGGAGATCAAAGGCGGTGGAGGGT
CCGGCGGTGGTGGCTCCGGACGGTCGCTGGTGGAGTCTGGGGGAGGCTTGGTCCAGCCTGGGGG
GTCCCTGAGACTCTCCTGTACTGCCTCTGGATTCACCATCAGTAGCTACCACATGCAGTGGGTC
CGCCAGGCTCCAGGGAAGGGGCTGGAGTACATCGGAACCATTAGTAGTGGTGGTAATGTATACT
ACGCAAGCTCCGCTAGAGGCAGATTCACCATCTCCAGACCCTCGTCCAAGAACACGGTGGATCT
TCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCTTCAGGCGGTGGCGGTAGTGGGG
GAGGCGGTTCTGGCGGCGGAGGGTCCGGCGGTGGAGGATCAGACGTTGTGATGACCCAGTCTCC
ATCTTCCGTGTCTGCATCTGTAGGAGACAGAGTCACCATCACCTGTCAGGCCAGTCAGAACATT
AGGACTTACTTATCCTGGTATCAGCAGAAACCAGGGAAAGCCCCTAAGCTCCTGATCTATGCTG
CAGCCAATCTGGCATCTGGGGTCCCATCAAGGTTCAGCGGCAGTGGATCTGGGACAGATTTCAC
TCTCACCATCAGCGACCTGGAGCCTGGCGATGCTGCAACTTACTATTGTCAGTCTACCTATCTT
GGTACTGATTATGTTGGCGGTGCTTTCGGCGGAGGGACCAAGGTGGAGATCAAATGA
>Sequence ID 59: SI-55H12 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLSLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DVVMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGGGTKVEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGECGGGGSGGGGSGGGGSQSALTQPASVSGSPGQSITISC
TGTSSDVGGYNFVSWYQQHPGKAPKLMIYDVSDRPSGVSDRFSGSKSGNTASLIISGLQADDEA
DYYCSSYGSSSTHVIFGGGTKVTVLGGGGSGGGGSGGGGSGGGGSQVQLQESGGGLVKPGGSLS
LSCAASGFTFSSYWMSWVRQAPGKGLEWVANINRDGSASYYVDSVKGRFTISRDDAKNSLYLQM
NSLRAEDTAVYYCARDRGVGYFDLWGRGTLVTVSS
>Sequence ID 60: SI-55H12 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTAGG
CGGTGGCGGTAGTGGGGGAGGCGGTTCTGGCGGCGGAGGGTCCGGCGGTGGAGGATCACAGGTC
ACATTGAAGGAATCTGGCCCCGGCCTTGTTCAGCCAGGACAGACCCTTAGCCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCTCGGATGGAACTCTGGTCTTACTACTTTGATTA
TTGGGGGCAGGGGACTCTCGTCACGGTCTCGAGTGGCGGTGGAGGGTCCGGCGGTGGTGGATCA
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAAGGCTATTTTTATTTTATTAGTCGTACTTATGTAAATTCTTTCGGCGGCGGGAC
CAAGGTGGAGATCAAACGTACGGTGGCTGCACCATCTGTCTTCATCTTCCCGCCATCTGATGAG
CAGTTGAAATCTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAGCT
TCAACAGGGGAGAGTGTGGCGGTGGCGGTAGCGGTGGCGGCGGAAGTGGTGGCGGAGGATCCCA
GTCTGCCCTGACTCAGCCTGCCTCCGTGTCTGGGTCTCCTGGACAGTCGATCACCATCTCCTGC
ACTGGAACCAGCAGTGACGTTGGTGGTTATAACTTTGTCTCCTGGTACCAACAACACCCAGGCA
AAGCCCCCAAACTCATGATCTATGATGTCAGTGATCGGCCCTCAGGGGTGTCTGATCGCTTCTC
CGGCTCCAAGTCTGGCAACACGGCCTCCCTGATCATCTCTGGCCTCCAGGCTGACGACGAGGCT
GATTATTACTGCAGCTCATATGGGAGCAGCAGCACTCATGTGATTTTCGGCGGAGGGACCAAGG
TGACCGTCCTAGGTGGAGGCGGTTCAGGCGGAGGTGGTTCCGGCGGTGGCGGCTCCGGTGGAGG
CGGCTCTCAGGTGCAATTGCAGGAGTCGGGGGGAGGCCTGGTCAAGCCTGGAGGGTCCCTGAGT
CTCTCCTGTGCAGCCTCTGGATTCACCTTTAGTAGTTATTGGATGAGCTGGGTCCGCCAGGCTC
CAGGGAAGGGGCTGGAGTGGGTGGCCAACATAAACCGCGATGGAAGTGCGAGTTACTATGTGGA
CTCTGTGAAGGGCCGATTCACCATCTCCAGAGACGACGCCAAGAACTCACTGTATCTGCAAATG
AACAGCCTGAGAGCTGAGGACACGGCTGTGTATTACTGTGCGAGAGATCGTGGGGTGGGCTACT
TCGATCTCTGGGGCCGTGGCACCCTGGTCACCGTCTCTAGCTGA
>Sequence ID 61: SI-77H4 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGQGTKLTVLGGGGSGGGGSGGGGSG
GGGSEVQLVESGGGLVQPGGSLRLSCAASGFTISTNAMSWVRQAPGKGLEWVGVITGRDITYYA
SWAKGRFTISRDTSKNTVYLQMNSLRAEDTAVYYCARDGGSSAITSNNIWGQGTLVTVSSGGGG
SGGGGSQVQLQQSGPGLVKPSETLSITCTVSGFSLTNYGVHWIRQAPGKCLEWLGVIWSGGNTD
YNTPFTSRFTITKDNSKNQVYFKLRSVRADDTAIYYCARALTYYDYEFAYWGQGTLVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGAPSVFLFPPKPK
DTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQKS
LSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGGGTKVEIKGGGGSGGGGSGRSLVESGGGLVQPGGSLRLSCTASGFTISSYHMQWVRQAPG
KGLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGGGTKVEIK
>Sequence ID 62: SI-77H4 heavy chain nucleotide sequence
GAAATCGTTATGACGCAGAGTCCCTCCACGCTCTCCGCTAGTGTCGGGGATCGCGTCATTATCA
CATGCCAGGCCTCCGAGTCAATCAGCAGCTGGCTTGCATGGTATCAACAGAAGCCGGGAAAAGC
TCCTAAATTGCTGATCTATGAAGCGTCAAAATTGGCGTCTGGTGTCCCATCTAGGTTCTCCGGC
TCTGGGTCTGGTGCGGAATTTACTTTGACAATCTCCAGTCTTCAACCAGACGATTTCGCTACCT
ACTACTGCCAAGGGTATTTCTATTTTATAAGCCGGACATATGTAAACTCCTTCGGCCAAGGAAC
AAAGTTGACTGTTCTTGGTGGCGGAGGCAGTGGTGGCGGGGGCAGCGGAGGTGGTGGTTCAGGG
GGTGGTGGGAGCGAAGTCCAATTGGTAGAAAGTGGCGGTGGTCTGGTGCAACCTGGTGGATCTC
TTCGCCTCTCATGCGCCGCTAGTGGCTTTACTATTTCAACTAATGCGATGAGCTGGGTTCGCCA
GGCCCCCGGCAAAGGACTTGAGTGGGTCGGCGTCATCACCGGCAGGGACATTACATACTATGCG
AGTTGGGCAAAGGGCAGGTTCACGATTAGCCGCGATACTTCAAAGAATACCGTTTACCTTCAAA
TGAATAGCTTGAGGGCGGAAGACACAGCTGTGTATTACTGCGCGAGGGATGGAGGTAGTTCCGC
CATAACTTCCAACAACATATGGGGACAAGGCACGCTGGTTACTGTCTCGAGTGGCGGTGGAGGG
TCCGGCGGTGGTGGATCACAAGTACAGTTGCAGCAATCCGGTCCCGGTCTCGTCAAACCGAGTG
AGACGCTTAGTATAACGTGTACTGTTTCAGGCTTTAGCCTTACGAACTATGGAGTTCACTGGAT
TCGGCAGGCACCCGGCAAATGTTTGGAATGGCTGGGTGTTATTTGGTCAGGTGGAAATACAGAC
TATAACACCCCCTTTACAAGTCGGTTCACAATTAGGAAAGATAATTCCAAAAATCAAGTTTATT
TCAAGTTGAGATCCGTCCGCGCGGACGACACTGCGATCTACTATTGTGCGAGGGCACTGACCTA
CTACGATTACGAATTTGCGTATTGGGGGCAAGGGACTCTTGTAACAGTCTCCAGTGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTGTCGAGCGGTGGAGGCGGATCTGGCGGAGGTGGTTCCGGCG
GTGGCGGCTCCGGTGGAGGCGGCTCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCGGAGGGACCAAGGTGGAGATCAAAGGCGGTGGAGGGTCCGGCGGTGGTGGCT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGCCAGGCTCCAGGG
AAGGGGCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTCAGGCCAGTCAGAACATTAGGACTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAATGA
>Sequence ID 63: SI-77H4 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLSLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGCGTKLTVLRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGECGGGGSGGGGSGGGGSQSALTQPASVSGSPGQSITISCTGTSS
DVGGYNFVSWYQQHPGKAPKLMIYDVSDRPSGVSDRFSGSKSGNTASLIISGLQADDEADYYCS
SYGSSSTHVIFGGGTKVTVLGGGGSGGGGSGGGGSGGGGSQVQLQESGGGLVKPGGSLSLSCAA
SGFTFSSYWMSWVRQAPGKGLEWVANINRDGSASYYVDSVKGRFTISRDDAKNSLYLQMNSLRA
EDTAVYYCARDRGVGYFDLWGRGTLVTVSS
>Sequence ID 64: SI-77H4 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTAGG
CGGTGGCGGTAGTGGGGGAGGCGGTTCTGGCGGCGGAGGGTCCGGCGGTGGAGGATCACAGGTC
ACATTGAAGGAATCTGGCCCCGGCCTTGTTCAGCCAGGACAGACCCTTAGCCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCTCGGATGGAACTCTGGTCTTACTACTTTGATTA
TTGGGGGCAGGGGACTCTCGTCACGGTCTCGAGTGGCGGTGGAGGGTCCGGCGGTGGTGGATCA
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGATGCGGTACAAAGCTGACCGTTTT
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTGGCGGTGGCGGTAGCGGTGGCGGCGGAAGTGGTGGCGGAGGATCCCAGTCTGCCCTGACTCA
GCCTGCCTCCGTGTCTGGGTCTCCTGGACAGTCGATCACCATCTCCTGCACTGGAACCAGCAGT
GACGTTGGTGGTTATAACTTTGTCTCCTGGTACCAACAACACCCAGGCAAAGCCCCCAAACTCA
TGATCTATGATGTCAGTGATCGGCCCTCAGGGGTGTCTGATCGCTTCTCCGGCTCCAAGTCTGG
CAACACGGCCTCCCTGATCATCTCTGGCCTCCAGGCTGACGACGAGGCTGATTATTACTGCAGC
TCATATGGGAGCAGCAGCACTCATGTGATTTTCGGCGGAGGGACCAAGGTGACCGTCCTAGGTG
GAGGCGGTTCAGGCGGAGGTGGTTCCGGCGGTGGCGGCTCCGGTGGAGGCGGCTCTCAGGTGCA
ATTGCAGGAGTCGGGGGGAGGCCTGGTCAAGCCTGGAGGGTCCCTGAGTCTCTCCTGTGCAGCC
TCTGGATTCACCTTTAGTAGTTATTGGATGAGCTGGGTCCGCCAGGCTCCAGGGAAGGGGCTGG
AGTGGGTGGCCAACATAAACCGCGATGGAAGTGCGAGTTACTATGTGGACTCTGTGAAGGGCCG
ATTGAGCATCTCCAGAGACGAGGCCAAGAACTGAGTGTATCTGCAAATGAACAGCCTGAGAGCT
GAGGACACGGCTGTGTATTACTGTGCGAGAGATCGTGGGGTGGGCTACTTCGATCTCTGGGGCC
GTGGCACCCTGGTCACCGTCTCTAGCTGA
>Sequence ID 65: SI-77H5 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGQGTKLTVLGGGGSGGGGSGGGGSG
GGGSEVQLVESGGGLVQPGGSLRLSCAASGFTISTNAMSWVRQAPGKGLEWVGVITGRDITYYA
SWAKGRFTISRDTSKNTVYLQMNSLRAEDTAVYYCARDGGSSAITSNNIWGQGTLVTVSSGGGG
SGGGGSQVQLKQSGPGLVQPSQSLSITCTVSGFSLTNYGVHWVRQSPGKCLEWLGVIWSGGNTD
YNTPFTSRLSINKDNSKSQVFFKMNSLQSNDTAIYYCARALTYYDYEFAYWGQGTLVTVSSAST
KGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEAAGAPSVFLFPPKPK
DTLMISRTPEVTCVVVDVSHEDPEVKFNWYVDGVEVHNAKTKPREEQYNSTYRVVSVLTVLHQD
WLNGKEYKCAVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHNHYTQKS
LSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLW
GQGTLVTVSSGGGGSGGGGSGGGGSGGGGSDIQMTQSPSTLSASVGDRVTITCQASQSISSHLN
WYQQKPGKAPKLLIYKASTLASGVPSRFSGSGSGTEFTLTISSLQPDDFATYYCQQGYSWGNVD
NVFGGGTKVEIKGGGGSGGGGSGRSLVESGGGLVQPGGSLRLSCTASGFTISSYHMQWVRQAPG
KGLEYIGTISSGGNVYYASSARGRFTISRPSSKNTVDLQMNSLRAEDTAVYYCARDSGYSDPMW
GQGTLVTVSSGGGGSGGGGSGGGGSGGGGSDVVMTQSPSSVSASVGDRVTITCQASQNIRTYLS
WYQQKPGKAPKLLIYAAANLASGVPSRFSGSGSGTDFTLTISDLEPGDAATYYCQSTYLGTDYV
GGAFGGGTKVEIK
>Sequence ID 66: SI-77H5 heavy chain nucleotide sequence
GAAATCGTTATGACGCAGAGTCCCTCCACGCTCTCCGCTAGTGTCGGGGATCGCGTCATTATCA
CATGCCAGGCCTCCGAGTCAATCAGCAGCTGGCTTGCATGGTATCAACAGAAGCCGGGAAAAGC
TCCTAAATTGCTGATCTATGAAGCGTCAAAATTGGCGTCTGGTGTCCCATCTAGGTTCTCCGGC
TCTGGGTCTGGTGCGGAATTTACTTTGACAATCTCCAGTCTTCAACCAGACGATTTCGCTACCT
ACTACTGCCAAGGGTATTTCTATTTTATAAGCCGGACATATGTAAACTCCTTCGGCCAAGGAAC
AAAGTTGACTGTTCTTGGTGGCGGAGGCAGTGGTGGCGGGGGCAGCGGAGGTGGTGGTTCAGGG
GGTGGTGGGAGCGAAGTCCAATTGGTAGAAAGTGGCGGTGGTCTGGTGCAACCTGGTGGATCTC
TTCGCCTCTCATGCGCCGCTAGTGGCTTTACTATTTCAACTAATGCGATGAGCTGGGTTCGCCA
GGCCCCCGGCAAAGGACTTGAGTGGGTCGGCGTCATCACCGGCAGGGACATTACATACTATGCG
AGTTGGGCAAAGGGCAGGTTCACGATTAGCCGCGATACTTCAAAGAATACCGTTTACCTTCAAA
TGAATAGCTTGAGGGCGGAAGACACAGCTGTGTATTACTGCGCGAGGGATGGAGGTAGTTCCGC
CATAACTTCCAACAACATATGGGGACAAGGCACGCTGGTTACTGTCTCGAGTGGCGGTGGAGGG
TCCGGCGGTGGTGGATCACAGGTGCAGCTGAAGCAGTCAGGACCTGGCCTAGTGCAGCCCTCAC
AGAGCCTGTCCATCACCTGCACAGTCTCTGGTTTCTCATTAACTAACTATGGTGTACACTGGGT
TCGCCAGTCTCCAGGAAAGTGTCTGGAGTGGCTGGGAGTGATATGGAGTGGTGGAAACACAGAC
TATAATACACCTTTCACATCCAGACTGAGCATCAACAAGGACAATTCCAAGAGCCAAGTTTTCT
TTAAAATGAACAGTCTGCAATCTAATGACACAGCCATATATTACTGTGCCAGAGCCCTCACCTA
CTATGATTACGAGTTTGCTTACTGGGGCCAAGGGACTCTGGTCACTGTCTCGAGTGCTAGCACC
AAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCT
GACCAGCGGCGTGCACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAACGTGAATCACAAGC
CCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAG
GACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGAC
TGGCTGAATGGCAAGGAGTACAAGTGCGCGGTCTCCAACAAAGCCCTCCCAGCCCCCATCGAGA
AAACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTGC
TGGACTCCGACGGCTCCTTCTTCCTCTATAGCAAGCTCACCGTGGACAAGAGCAGGTGGCAGCA
GGGGAACGTCTTCTCATGCTCCGTGATGCATGAGGCTCTGCACAACCACTACACGCAGAAGAGC
CTCTCCCTGTCTCCGGGTGGCGGTGGAGGGTCCGGCGGTGGTGGATCCGAGGTGCAGCTGTTGG
AGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGT
TCACCATCTCCAGAGACAATTCCAAGAACACGCTGTATCTGCAAATGAACAGCCTGAGAGCCGA
GGACACGGCCGTATATTACTGTGCGAGATCGGCGTTTTCGTTCGACTACGCCATGGACCTCTGG
GGCCAGGGAACCCTGGTCACCGTGTCGAGCGGTGGAGGCGGATCTGGCGGAGGTGGTTCCGGCG
GTGGCGGCTCCGGTGGAGGCGGCTCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAAC
TGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAG
CCTGCAGCCTGATGATTTTGCAACTTATTACTGCCAACAGGGTTATAGTTGGGGTAATGTTGAT
AATGTTTTCGGCGGAGGGACCAAGGTGGAGATCAAAGGCGGTGGAGGGTCCGGCGGTGGTGGCT
CCGGACGGTCGCTGGTGGAGTCTGGGGGAGGCTTGGTCCAGCCTGGGGGGTCCCTGAGACTCTC
CTGTACTGCCTCTGGATTCACCATCAGTAGCTACCACATGCAGTGGGTCCGCCAGGCTCCAGGG
AAGGGGCTGGAGTACATCGGAACCATTAGTAGTGGTGGTAATGTATACTACGCAAGCTCCGCTA
GAGGCAGATTCACCATCTCCAGACCCTCGTCCAAGAACACGGTGGATCTTCAAATGAACAGCCT
GAGAGCCGAGGACACGGCTGTGTATTACTGTGCGAGAGACTCTGGTTATAGTGATCCTATGTGG
GGCCAGGGAACCCTGGTCACCGTCTCTTCAGGCGGTGGCGGTAGTGGGGGAGGCGGTTCTGGCG
GCGGAGGGTCCGGCGGTGGAGGATCAGACGTTGTGATGACCCAGTCTCCATCTTCCGTGTCTGC
ATCTGTAGGAGACAGAGTCACCATCACCTGTGAGGCGAGTCAGAACATTAGGAGTTACTTATCC
TGGTATCAGCAGAAACCAGGGAAAGCCCCTAAGCTCCTGATCTATGCTGCAGCCAATCTGGCAT
CTGGGGTCCCATCAAGGTTCAGCGGCAGTGGATCTGGGACAGATTTCACTCTCACCATCAGCGA
CCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAATGA
>Sequence ID 67: SI-77H5 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVLGGGGSGGGGSGGGGSGGGGSQV
TLKESGPGLVQPGQTLSLTCAFSGFSLSTSGMGVGWIRQPPGKGLEWLAHIWWDDDKRYNPALK
SRLTISKDTSKNQVYLQMNSLDAEDTAVYYCARMELWSYYFDYWGQGTLVTVSSGGGGSGGGGS
DILLTQSPVILSVSPGERVSFSCRASQSIGTNIHWYQQRTNGSPRLLIKYASESISGIPSRFSG
SGSGTDFTLSINSVESEDIADYYCQQNNNWPTTFGCGTKLELKRTVAAPSVFIFPPSDEQLKSG
TASVVCLLNNFYPREAKVQWKVDNALQSGNSQESVTEQDSKDSTYSLSSTLTLSKADYEKHKVY
ACEVTHQGLSSPVTKSFNRGECGGGGSGGGGSGGGGSQSALTQPASVSGSPGQSITISCTGTSS
DVGGYNFVSWYQQHPGKAPKLMIYDVSDRPSGVSDRFSGSKSGNTASLIISGLQADDEADYYCS
SYGSSSTHVIFGGGTKVTVLGGGGSGGGGSGGGGSGGGGSQVQLQESGGGLVKPGGSLSLSCAA
SGFTFSSYWMSWVRQAPGKGLEWVANINRDGSASYYVDSVKGRFTISRDDAKNSLYLQMNSLRA
EDTAVYYCARDRGVGYFDLWGRGTLVTVSS
>Sequence ID 68: SI-77H5 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTAGG
CGGTGGCGGTAGTGGGGGAGGCGGTTCTGGCGGCGGAGGGTCCGGCGGTGGAGGATCACAGGTC
ACATTGAAGGAATCTGGCCCCGGCCTTGTTCAGCCAGGACAGACCCTTAGCCTCACCTGTGCCT
TCAGTGGTTTTTCTCTTAGCACTAGCGGTATGGGGGTCGGCTGGATTCGGCAGCCTCCCGGCAA
AGGTCTTGAGTGGTTGGCTCACATTTGGTGGGACGACGACAAACGGTATAATCCTGCCTTGAAA
AGTCGGCTGACCATTAGTAAGGATACCTCAAAAAATCAAGTGTACTTGCAAATGAATAGCCTTG
ACGCCGAGGATACGGCTGTATATTATTGCGCTCGGATGGAACTCTGGTCTTACTACTTTGATTA
TTGGGGGCAGGGGACTCTCGTCACGGTCTCGAGTGGCGGTGGAGGGTCCGGCGGTGGTGGATCA
GACATCTTGCTGACTCAGTCTCCAGTCATCCTGTCTGTGAGTCCAGGAGAAAGAGTCAGTTTCT
CCTGCAGGGCCAGTCAGAGTATTGGCACAAACATACACTGGTATCAGCAAAGAACAAATGGTTC
TCCAAGGCTTCTCATAAAGTATGCTTCTGAGTCTATCTCTGGGATTCCTTCCAGGTTTAGTGGC
AGTGGATCAGGGACAGATTTTACTCTTAGCATCAACAGTGTGGAGTCTGAAGATATTGCAGATT
ATTACTGTCAACAAAATAATAACTGGCCAACCACGTTCGGTTGTGGGACCAAGCTGGAGCTGAA
ACGTACGGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGGGAGAGT
GTGGCGGTGGCGGTAGCGGTGGCGGCGGAAGTGGTGGCGGAGGATCCCAGTCTGCCCTGACTCA
GCCTGCCTCCGTGTCTGGGTCTCCTGGACAGTCGATCACCATCTCCTGCACTGGAACCAGCAGT
GACGTTGGTGGTTATAACTTTGTCTCCTGGTACCAACAACACCCAGGCAAAGCCCCCAAACTCA
TGATCTATGATGTCAGTGATCGGCCCTCAGGGGTGTCTGATCGCTTCTCCGGCTCCAAGTCTGG
CAACACGGCCTCCCTGATCATCTCTGGCCTCCAGGCTGACGACGAGGCTGATTATTACTGCAGC
TCATATGGGAGCAGCAGCACTCATGTGATTTTCGGCGGAGGGACCAAGGTGACCGTCCTAGGTG
GAGGCGGTTCAGGCGGAGGTGGTTCCGGCGGTGGCGGCTCCGGTGGAGGCGGCTCTCAGGTGCA
ATTGCAGGAGTCGGGGGGAGGCCTGGTCAAGCCTGGAGGGTCCCTGAGTCTCTCCTGTGCAGCC
TCTGGATTCACCTTTAGTAGTTATTGGATGAGCTGGGTCCGCCAGGCTCCAGGGAAGGGGCTGG
AGTGGGTGGCCAACATAAACCGCGATGGAAGTGCGAGTTACTATGTGGACTCTGTGAAGGGCCG
ATTGAGCATCTCCAGAGACGAGGCCAAGAACTGAGTGTATCTGCAAATGAACAGCCTGAGAGCT
GAGGACACGGCTGTGTATTACTGTGCGAGAGATCGTGGGGTGGGCTACTTCGATCTCTGGGGCC
GTGGCACCCTGGTCACCGTCTCTAGCTGA
>Sequence ID 69: αEGFR H1 VH amino acid sequence
EVQLVESGGGLVQPGGSLRLSCKVSGFSLTNYGVHWVRQAPGKGLEWVGVIWSGGNTDYNTPFT
SRFTISRDTSKNTVYLQMNSLRAEDTAVYYCARALTYYDYEFAYWGQGTLVTVSS
>Sequence ID 70: αEGFR H1 VH nucleotide sequence
GAAGTTCAGCTGGTGGAATCCGGCGGAGGATTGGTTCAACCTGGCGGCTCTCTGAGACTGTCCT
GTAAGGTGTCTGGCTTCTCCCTGACCAACTACGGCGTGCACTGGGTCCGACAGGCACCTGGAAA
AGGACTGGAATGGGTCGGAGTGATTTGGAGCGGCGGCAACACCGACTACAACACCCCTTTCACC
AGCCGGTTCACCATCTCTCGGGACACCTCCAAGAACACCGTGTACCTGCAGATGAACTCCCTGA
GAGCCGAGGACACCGCCGTGTACTATTGTGCTAGAGCCCTGACCTACTATGACTACGAGTTCGC
CTATTGGGGCCAGGGAACCCTGGTCACAGTCTCCTCT
>Sequence ID 71: αEGFR H1 VL amino acid sequence
EIVMTQSPSTLSASVGDRVIITCRASQSIGTNIHWYQQKPGKAPKLLIYYASESISGIPSRFSG
SGSGAEFTLTISSLQPDDFATYYCQQNNNWPTTFGQGTKLTVL
>Sequence ID 72: αEGFR H1 VL nucleotide sequence
GAGATCGTGATGACCCAGTCTCCTTCCACACTGTCCGCCTCTGTGGGCGACAGAGTGATCATCA
CCTGTAGAGCCAGCCAGTCCATCGGCACCAACATCCACTGGTATCAGCAGAAGCCTGGCAAGGC
CCCTAAGCTGCTGATCTACTACGCCTCCGAGTCTATCAGCGGCATCCCCTCCAGATTCTCCGGC
TCTGGATCTGGCGCTGAGTTTACCCTGACAATCTCCAGCCTGCAGCCTGACGACTTCGCCACCT
ACTACTGCCAGCAGAACAACAACTGGCCCACCACCTTTGGCCAGGGCACCAAACTGACAGTTCT
T
>Sequence ID 73: αEGFR H4 VH amino acid sequence
QVQLQQSGPGLVKPSETLSITCTVSGFSLTNYGVHWIRQAPGKGLEWLGVIWSGGNTDYNTPFT
SRFTITKDNSKNQVYFKLRSVRADDTAIYYCARALTYYDYEFAYWGQGTLVTVSS
>Sequence ID 74: CEGFR H4 VH nucleotide sequence
CAAGTTCAGTTGCAGCAGTCTGGCCCTGGCCTGGTCAAGCCTTCTGAGACACTGTCCATCACCT
GTACCGTGTCCGGCTTCTCCCTGACCAATTACGGCGTGCACTGGATCAGACAGGCCCCTGGCAA
AGGACTGGAATGGCTGGGAGTGATTTGGAGCGGCGGCAACACCGACTACAACACCCCTTTCACC
AGCCGGTTCACCATCACCAAGGACAACTCCAAGAACCAGGTGTACTTCAAGCTGCGGAGCGTGC
GGGCTGATGACACCGCCATCTACTACTGTGCTCGGGCCCTGACCTACTACGACTACGAGTTTGC
TTACTGGGGCCAGGGCACCCTGGTCACAGTTTCTTCT
>Sequence ID 75: αEGFR H4 VL amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLELK
>Sequence ID 76: αEGFR H4 VL nucleotide sequence
GAGATCGTGCTGACCCAGTCTCCTTCCACACTGTCTGTGTCTCCCGGCGAGAGAGCCACCTTCA
GCTGTAGAGCCTCTCAGTCCATCGGCACCAACATCCACTGGTATCAGCAGAAGCCCGGCAAGCC
TCCTCGGCTGCTGATTAAGTACGCCTCCGAGTCCATCAGCGGCATCCCTGACAGATTCTCCGGC
TCTGGCTCTGGCACCGAGTTTACCCTGACCATCTCCTCCGTGCAGTCCGAGGATTTCGCCGTGT
ACTACTGCCAGCAGAACAACAACTGGCCCACCACCTTTGGACCCGGCACCAAGCTGGAATTGAA
A
>Sequence ID 77: αEGFR H7 VH amino acid sequence
QVQLQQSGPGLVKPSETLSITCTVSGFSLTNYGVHWIRQAPGKGLEWLGVIWSGGNTDYNTPFT
SRFTITKDNSKNQVYFKLRSVRADDTAIYYCARALTYYDYEFAYWGQGTLVTVSS
>Sequence ID 78: αEGFR H7 VH nucleotide sequence
CAAGTTCAGTTGCAGCAGTCTGGCCCTGGCCTGGTCAAGCCTTCTGAGACACTGTCCATCACCT
GTACCGTGTCCGGCTTCTCCCTGACCAATTACGGCGTGCACTGGATCAGACAGGCCCCTGGCAA
AGGACTGGAATGGCTGGGAGTGATTTGGAGCGGCGGCAACACCGACTACAACACCCCTTTCACC
AGCCGGTTCACCATCACCAAGGACAACTCCAAGAACCAGGTGTACTTCAAGCTGCGGAGCGTGC
GGGCTGATGACACCGCCATCTACTACTGTGCTCGGGCCCTGACCTACTACGACTACGAGTTTGC
TTACTGGGGCCAGGGCACCCTGGTCACAGTTTCTTCT
>Sequence ID 79: αEGFR H7 VL amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGPGTKLTVL
>Sequence ID 80: αEGFR H7 VL nucleotide sequence
GAGATCGTGCTGACCCAGTCTCCTTCCACACTGTCTGTGTCTCCCGGCGAGAGAGCCACCTTCA
GCTGTAGAGCCTCTCAGTCCATCGGCACCAACATCCACTGGTATCAGCAGAAGCCCGGCAAGCC
TCCTCGGCTGCTGATTAAGTACGCCTCCGAGTCCATCAGCGGCATCCCTGACAGATTCTCCGGC
TCTGGCTCTGGCACCGAGTTTACCCTGACCATCTCCTCCGTGCAGTCCGAGGATTTCGCCGTGT
ACTACTGCCAGCAGAACAACAACTGGCCCACCACCTTTGGACCCGGCACCAAGCTGACAGTTCT
T
>Sequence ID 81: αEGFR H7 VH staple amino acid sequence
QVQLQQSGPGLVKPSETLSITCTVSGFSLTNYGVHWIRQAPGKCLEWLGVIWSGGNTDYNTPFT
SRFTITKDNSKNQVYFKLRSVRADDTAIYYCARALTYYDYEFAYWGQGTLVTVSS
>Sequence ID 82: αEGFR H7 VH staple nucleotide sequence
CAAGTACAGTTGCAGCAATCCGGTCCCGGTCTCGTCAAACCGAGTGAGACGCTTAGTATAACGT
GTACTGTTTCAGGCTTTAGCCTTACGAACTATGGAGTTCACTGGATTCGGCAGGCACCCGGCAA
ATGTTTGGAATGGCTGGGTGTTATTTGGTCAGGTGGAAATACAGACTATAACACCCCCTTTACA
AGTCGGTTCACAATTACGAAAGATAATTCCAAAAATCAAGTTTATTTCAAGTTGAGATCCGTCC
GCGCGGACGACACTGCGATCTACTATTGTGCGAGGGCACTGACCTACTACGATTACGAATTTGC
GTATTGGGGGCAAGGGACTCTTGTAACAGTCTCCAGT
>Sequence ID 83: αEGFR H7 VL staple amino acid sequence
EIVLTQSPSTLSVSPGERATFSCRASQSIGTNIHWYQQKPGKPPRLLIKYASESISGIPDRFSG
SGSGTEFTLTISSVQSEDFAVYYCQQNNNWPTTFGCGTKLTVL
>Sequence ID 84: αEGFR H7 VL staple nucleotide sequence
GAAATCGTCCTTACACAATCTCCTAGCACACTGAGTGTGAGCCCCGGCGAACGCGCGACTTTCT
CTTGCAGGGCAAGTCAATCCATAGGGACTAATATACATTGGTATCAACAAAAGCCAGGTAAACC
ACCCAGGCTTTTGATTAAGTATGCAAGTGAGTCTATTTCCGGTATCCCTGACCGCTTCTCTGGA
TCAGGCAGTGGCACAGAGTTCACACTCACCATATCTAGTGTGCAATCAGAGGACTTCGCCGTGT
ATTACTGCCAACAGAATAATAACTGGCCGACTACCTTCGGATGCGGTACAAAGCTGACCGTTTT
A
>Sequence ID 85: αCD19 SI-huBU12 H1 VH amino acid sequence
QVTLKESGPGLVQPGQTLSLTCAFSGFSLSTSGMGVGWIRQPPGKGLEWLAHIWWDDDKRYNPA
LKSRLTISKDTSKNQVYLQMNSLDAEDTAVYYCARMELWSYYFDYWGQGTLVTVSS
>Sequence ID 86: αCD19 SI-huBU12 H1 VH nucleotide sequence
CAGGTCACATTGAAGGAATCTGGCCCCGGCCTTGTTCAGCCAGGACAGACCCTTAGCCTCACCT
GTGCCTTCAGTGGTTTTTCTCTTAGCACTAGCGGTATGGGGGTCGGCTGGATTCGGCAGCCTCC
CGGCAAAGGTCTTGAGTGGTTGGCTCACATTTGGTGGGACGACGACAAACGGTATAATCCTGCC
TTGAAAAGTCGGCTGACCATTAGTAAGGATACCTCAAAAAATCAAGTGTACTTGCAAATGAATA
GCCTTGACGCCGAGGATACGGCTGTATATTATTGCGCTCGGATGGAACTCTGGTCTTACTACTT
TGATTATTGGGGGCAGGGGACTCTCGTCACGGTCTCGAGT
>Sequence ID 87: αCD19 SI-huBU12 H1 VL amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKVTVL
>Sequence ID 88: αCD19 SI-huBU12 H1 VL nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAAGTGACCGTCCTA
>Sequence ID 89: αCD3 284A10 staple VH amino acid sequence
EVQLVESGGGLVQPGGSLRLSCAASGFTISTNAMSWVRQAPGKCLEWIGVITGRDITYYASWAK
GRFTISRDNSKNTLYLQMNSLRAEDTAVYYCARDGGSSAITSNNIWGQGTLVTVST
>Sequence ID 90: αCD3 284A10 staple VH nucleotide sequence
GAGGTGCAGCTGGTGGAGTCTGGGGGAGGCTTGGTCCAGCCTGGGGGGTCCCTGAGACTCTCCT
GTGCAGCCTCTGGATTCACCATCAGTACCAATGCAATGAGCTGGGTCCGCCAGGCTCCAGGGAA
GTGCCTGGAGTGGATCGGAGTCATTACTGGTCGTGATATCACATACTACGCGAGCTGGGCGAAA
GGCAGATTCACCATCTCCAGAGACAATTCCAAGAACACGCTGTATCTTCAAATGAACAGCCTGA
GAGCCGAGGACACGGCTGTGTATTACTGTGCGAGAGACGGTGGTTCTTCTGCTATTACTAGTAA
CAACATTTGGGGCCAGGGAACCCTGGTCACCGTGTCGACA
>Sequence ID 91: αCD3 284A10 staple VL amino acid sequence
DNNMTQSPSTLSASVGDRVTINCQASESISSWLAWYQQKPGKAPKLLIYEASKLASGVPSRFSG
SGSGTEFTLTISSLQPDDFATYYCQGYFYFISRTYVNSFGCGTKVEIK
>Sequence ID 92: αCD3 284A10 staple VL nucleotide sequence
GACGTCGTGATGACCCAGTCTCCTTCCACCCTGTCTGCATCTGTAGGAGACAGAGTCACCATCA
ATTGCCAAGCCAGTGAGAGCATTAGCAGTTGGTTAGCCTGGTATCAGCAGAAACCAGGGAAAGC
CCCTAAGCTCCTGATCTATGAAGCATCCAAACTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAAGGCTATTTTTATTTTATTAGTCGTACTTATGTAAATTCTTTCGGCTGTGGGAC
CAAGGTGGAGATCAAA
>Sequence ID 93: αCD3 284A10 H1 VH amino acid sequence
EVQLVESGGGLVQPGGSLRLSCAASGFTISTNAMSWVRQAPGKGLEWVGVITGRDITYYASWAK
GRFTISRDTSKNTVYLQMNSLRAEDTAVYYCARDGGSSAITSNNIWGQGTLVTVSS
>Sequence ID 94: αCD3 284A10 H1 VH nucleotide sequence
GAAGTCCAATTGGTAGAAAGTGGCGGTGGTCTGGTGCAACCTGGTGGATCTCTTCGCCTCTCAT
GCGCCGCTAGTGGCTTTACTATTTCAACTAATGCGATGAGCTGGGTTCGCCAGGCCCCCGGCAA
AGGACTTGAGTGGGTCGGCGTCATCACCGGCAGGGACATTACATACTATGCGAGTTGGGCAAAG
GGCAGGTTCACGATTAGCCGCGATACTTCAAAGAATACCGTTTACCTTCAAATGAATAGCTTGA
GGGCGGAAGACACAGCTGTGTATTACTGCGCGAGGGATGGAGGTAGTTCCGCCATAACTTCCAA
CAACATATGGGGACAAGGCACGCTGGTTACTGTCTCGAGT
>Sequence ID 95: αCD3 284A10 H1 VL amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGQGTKLTVL
>Sequence ID 96: αCD3 284A10 H1 VL nucleotide sequence
GAAATCGTTATGACGCAGAGTCCCTCCACGCTCTCCGCTAGTGTCGGGGATCGCGTCATTATCA
CATGCCAGGCCTCCGAGTCAATCAGCAGCTGGCTTGCATGGTATCAACAGAAGCCGGGAAAAGC
TCCTAAATTGCTGATCTATGAAGCGTCAAAATTGGCGTCTGGTGTCCCATCTAGGTTCTCCGGC
TCTGGGTCTGGTGCGGAATTTACTTTGACAATCTCCAGTCTTCAACCAGACGATTTCGCTACCT
ACTACTGCCAAGGGTATTTCTATTTTATAAGCCGGACATATGTAAACTCCTTCGGCCAAGGAAC
AAAGTTGACTGTTCTT
>Sequence ID 97: αCD3 284A10 H1 staple VH amino acid sequence
EVQLVESGGGLVQPGGSLRLSCTASGFTISTNAMSWVRQAPGKCLEWVGVITGRDITYYASWAK
GRFTISRDTSKNTVYLQMNSLRAEDTAVYYCARDGGSSAITSNNIWGQGTLVTVST
>Sequence ID 98: αCD3 284A10 H1 staple VH nucleotide sequence
GAGGTGCAGCTGGTGGAGAGCGGCGGCGGCCTGGTGCAGCCCGGCGGCAGCCTGAGGCTGAGCT
GCACCGCCAGCGGCTTCACCATCAGCACCAACGCCATGAGCTGGGTGAGGCAGGCCCCCGGCAA
GTGCCTGGAGTGGGTGGGCGTGATCACCGGCAGGGACATCACCTACTACGCCAGCTGGGCCAAG
GGCAGGTTCACCATCAGCAGGGACACCAGCAAGAACACCGTGTACCTGCAGATGAACAGCCTGA
GGGCCGAGGACACCGCCGTGTACTACTGCGCCAGGGACGGCGGCAGCAGCGCCATCACCAGCAA
CAACATCTGGGGCCAGGGCACCCTGGTGACCGTGTCGACA
>Sequence ID 99: αCD3 284A10 H1 staple VL amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVL
>Sequence ID 100: αCD3 284A10 H1 staple VL nucleotide sequence
GAGATCGTGATGACCCAGAGCCCCAGCACCCTGAGCGCCAGCGTGGGCGACAGGGTGATCATCA
CCTGCCAGGCCAGCGAGAGCATCAGCAGCTGGCTGGCCTGGTACCAGCAGAAGCCCGGCAAGGC
CCCCAAGCTGCTGATCTACGAGGCCAGCAAGCTGGCCAGCGGCGTGCCCAGCAGGTTCAGCGGC
AGCGGCAGCGGCGCCGAGTTCACCCTGACCATCAGCAGCCTGCAGCCCGACGACTTCGCCACCT
ACTACTGCCAGGGCTACTTCTACTTCATCAGCAGGACCTACGTGAACAGCTTCGGCTGCGGCAC
CAAGCTGACCGTGCTG
>Sequence ID 101: αCD3 283E3 H1 VH amino acid sequence
QVQLQESGGRLVQPGEPLSLTCKTSGIDLSSNAIGWVRQAPGKGLEWIGVIFGSGNTYYASWAK
GRFTISRSTSTVYLKMNSLRSEDTAIYYCARGGYSSDIWGQGTLVTVSS
>Sequence ID 102: αCD3 283E3 H1 VH nucleotide sequence
CAAGTGCAGTTGCAAGAAAGTGGTGGTAGACTGGTTCAGCCTGGTGAACCCTTGTCACTGACGT
GTAAAACAAGCGGCATTGATCTGTCCTCTAACGCCATCGGATGGGTCCGACAGGCCCCAGGAAA
AGGTCTGGAGTGGATCGGAGTTATCTTCGGGAGCGGCAATACATACTACGCAAGCTGGGCAAAA
GGGCGATTTACGATATCACGGAGCACCTCTACAGTTTATTTGAAAATGAACTCCCTCCGGTCCG
AGGATACCGCGATATATTACTGTGCCAGAGGGGGGTACTCCTCTGATATCTGGGGGCAGGGTAC
ACTGGTTACAGTTTCATCC
>Sequence ID 103: αCD3 283E3 H1 VL amino acid sequence
DPVLTQSPSSLSASVGDRVTISCQSSQSVAKNNNLAWFQQKPGQAPKLLIYSASTLAAGVPSRF
SGSGSGTDFTLTISSVQPEDFATYYCSARDSGNIQSFGGGTKVEIK
>Sequence ID 104: αCD3 283E3 H1 VL nucleotide sequence
GATCCAGTTCTGACACAAAGTCCATCCAGCCTGTCTGCCTCAGTCGGCGACAGAGTGACCATCA
GTTGCCAGAGCTCACAGTCTGTGGCTAAGAACAACAACTTGGCGTGGTTCCAACAGAAACCTGG
ACAGGCTCCGAAATTGCTGATCTATTCTGCTTCCACGCTTGCTGCTGGTGTTCCTTCCCGCTTT
TCAGGTAGTGGTAGCGGGACAGACTTCACTTTGACTATAAGCAGCGTGCAGCCTGAAGATTTTG
CGACCTACTATTGTTCTGCTAGAGACAGTGGAAATATTCAGTCCTTTGGGGGGGGAACGAAGGT
CGAAATAAAG
>Sequence ID 105: αPDL1 PL221G5 staple VH amino acid sequence
EVQLLESGGGLVQPGGSLRLSCAASGFSFSSGYDMCWVRQAPGKCLEWIACIAAGSAGITYDAN
WAKGRETISRDNSKNTLYLQMNSLRAEDTAVYYCARSAFSFDYAMDLWGQGTLVTVSS
>Sequence ID 106: αPDL1 PL221G5 staple VH nucleotide sequence
GAGGTGCAGCTGTTGGAGTCTGGGGGAGGCTTGGTACAGCCTGGGGGGTCCCTGAGACTCTCCT
GTGCAGCCTCTGGATTCTCCTTCAGTAGCGGGTACGACATGTGCTGGGTCCGCCAGGCTCCAGG
GAAGTGCCTGGAGTGGATCGCATGCATTGCTGCTGGTAGTGCTGGTATCACTTACGACGCGAAC
TGGGCGAAAGGCCGGTTCACCATCTCCAGAGACAATTCCAAGAACACGCTGTATCTGCAAATGA
ACAGCCTGAGAGCCGAGGACACGGCCGTATATTACTGTGCGAGATCGGCGTTTTCGTTCGACTA
CGCCATGGACCTCTGGGGCCAGGGAACCCTGGTCACCGTCTCGAGC
>Sequence ID 107: αPDL1 PL221G5 staple VL amino acid sequence
DIQMTQSPSTLSASVGDRVTITCQASQSISSHLNWYQQKPGKAPKLLIYKASTLASGVPSRFSG
SGSGTEFTLTISSLQPDDFATYYCQQGYSWGNVDNVFGCGTKVEIK
>Sequence ID 108: αPDL1 PL221G5 staple VL nucleotide sequence
GACATCCAGATGACCCAGTCTCCTTCCACCCTGTCTGCATCTGTAGGAGACAGAGTCACCATCA
CTTGCCAGGCCAGTCAGAGCATTAGTTCCCACTTAAACTGGTATCAGCAGAAACCAGGGAAAGC
CCCTAAGCTCCTGATCTATAAGGCATCCACTCTGGCATCTGGGGTCCCATCAAGGTTCAGCGGC
AGTGGATCTGGGACAGAATTTACTCTCACCATCAGCAGCCTGCAGCCTGATGATTTTGCAACTT
ATTACTGCCAACAGGGTTATAGTTGGGGTAATGTTGATAATGTTTTCGGCTGCGGGACCAAGGT
GGAGATCAAA
>Sequence ID 109: α41BB 466F6 staple VH amino acid sequence
RSLVESGGGLVQPGGSLRLSCTASGFTISSYHMQWVRQAPGKCLEYIGTISSGGNVYYASSARG
RFTISRPSSKNTVDLQMNSLRAEDTAVYYCARDSGYSDPMWGQGTLVTVSS
>Sequence ID 110: α41BB 466F6 staple VH nucleotide sequence
CGGTCGCTGGTGGAGTCTGGGGGAGGCTTGGTCCAGCCTGGGGGGTCCCTGAGACTCTCCTGTA
CTGCCTCTGGATTCACCATCAGTAGCTACCACATGCAGTGGGTCCGGCAGGCACCTGGGAAGTG
CCTGGAGTACATCGGAACCATTAGTAGTGGTGGTAATGTATACTACGCAAGCTCCGCTAGAGGC
AGATTCACCATCTCCAGACCCTCGTCCAAGAACACGGTGGATCTTCAAATGAACAGCCTGAGAG
CCGAGGACACGGCTGTGTATTACTGTGCGAGAGACTCTGGTTATAGTGATCCTATGTGGGGCCA
GGGAACCCTGGTCACCGTCTCTTCA
>Sequence ID 111: α41BB 466F6 staple VL amino acid sequence
DVVMTQSPSSVSASVGDRVTITCQASQNIRTYLSWYQQKPGKAPKLLIYAAANLASGVPSRFSG
SGSGTDFTLTISDLEPGDAATYYCQSTYLGTDYVGGAFGCGTKVEIK
>Sequence ID 112: α41BB 466F6 staple VL nucleotide sequence
GACGTTGTGATGACCCAGTCTCCATCTTCCGTGTCTGCATCTGTAGGAGACAGAGTCACCATCA
CCTGTCAGGCCAGTCAGAACATTAGGACTTACTTATCCTGGTATCAGCAGAAACCAGGGAAAGC
CCCTAAGCTCCTGATCTATGCTGCAGCCAATCTGGCATCTGGGGTCCCATCAAGGTTCAGCGGC
AGTGGATCTGGGACAGATTTCACTCTCACCATCAGCGACTTGGAACCTGGCGATGCTGCAACTT
ACTATTGTCAGTCTACCTATCTTGGTACTGATTATGTTGGCGGTGCTTTCGGCTGTGGGACCAA
GGTGGAGATCAAATGA
>Sequence ID 113: 4-1BB ligand trimer amino acid sequence
REGPELSPDDPAGLLDLRQGMFAQLVAQNVLLIDGPLSWYSDPGLAGVSLTGGLSYKEDTKELV
VAKAGVYYVFFQLELRRVVAGEGSGSVSLALHLQPLRSAAGAAALALTVDLPPASSEARNSAFG
FQGRLLHLSAGQRLGVHLHTEARARHAWQLTQGATVLGLFRVTPEIPAGLGSTGSGSKPGSGEG
STKGREGPELSPDDPAGLLDLRQGMFAQLVAQNVLLIDGPLSWYSDPGLAGVSLTGGLSYKEDT
KELVVAKAGVYYVFFQLELRRVVAGEGSGSVSLALHLQPLRSAAGAAALALTVDLPPASSEARN
SAFGFQGRLLHLSAGQRLGVHLHTEARARHAWQLTQGATVLGLFRVTPEIPAGLGGGGSGGGGS
REGPELSPDDPAGLLDLRQGMFAQLVAQNVLLIDGPLSWYSDPGLAGVSLTGGLSYKEDTKELV
VAKAGVYYVFFQLELRRVVAGEGSGSVSLALHLQPLRSAAGAAALALTVDLPPASSEARNSAFG
FQGRLLHLSAGQRLGVHLHTEARARHAWQLTQGATVLGLFRVTPEIPAGL
>Sequence ID 114: 4-1BB ligand trimer nucleotide sequence
CGAGAGGGCCCCGAGCTGTCTCCTGATGACCCAGCAGGCCTCTTGGACTTGCGGCAGGGTATGT
TCGCTCAACTTGTGGCTCAGAATGTTCTGCTCATTGATGGACCACTCTCTTGGTATAGTGACCC
CGGTCTGGCCGGGGTGAGTCTGACCGGCGGGCTCTCTTATAAAGAGGATACTAAGGAACTGGTC
GTAGCAAAAGCGGGCGTTTATTACGTTTTTTTTCAGCTGGAGCTCAGGCGCGTGGTGGCCGGCG
AGGGCAGTGGCTCTGTGTCCCTGGCCCTGCACTTACAGCCCTTGAGAAGCGCTGCAGGTGCTGC
CGCACTGGCTTTAACTGTTGACCTCCCTCCGGCCTCTTCTGAAGCTAGAAACAGCGCTTTCGGC
TTCCAAGGGCGCCTGCTGCACCTGAGCGCAGGCCAGCGCTTAGGTGTGCACCTTCATACAGAGG
CCAGGGCCCGACACGCTTGGCAGCTCACACAGGGTGCCACGGTTCTCGGACTTTTCCGCGTTAC
TCCCGAGATCCCCGCTGGCCTCGGAAGTACTGGTTCTGGGTCTAAACCCGGTTCCGGCGAAGGT
AGTACTAAAGGACGAGAAGGGCCAGAGTTAAGTCCAGATGACCCTGCTGGGCTTTTGGACCTGC
GGCAGGGCATGTTCGCTCAACTGGTGGCTCAGAACGTGCTGCTGATCGATGGCCCCCTGAGTTG
GTACAGCGATCCCGGGCTGGCAGGCGTGTCACTTACAGGGGGCCTCTCTTACAAGGAAGACACC
AAGGAGTTAGTGGTCGCTAAGGCTGGCGTGTATTACGTGTTCTTCCAACTGGAGCTGAGAAGGG
TTGTGGCAGGAGAGGGTAGCGGCAGCGTGTCTTTAGCCCTTCACTTGCAGCCCCTGAGGTCTGC
TGCAGGTGCAGCCGCTCTCGCGCTCACCGTGGATCTCCCCCCAGCCTCATCTGAAGCTAGGAAC
AGTGCATTTGGCTTTCAGGGACGCTTGCTGCACCTCTCCGCTGGACAGAGGCTGGGCGTGCACC
TTCACACAGAGGCCCGTGCCAGGCATGCATGGCAGCTCACTCAGGGGGCAACAGTGCTGGGTCT
CTTCCGCGTGACTCCTGAAATACCAGCTGGACTTGGCGGTGGAGGCAGCGGCGGAGGAGGATCT
CGTGAGGGGCCAGAACTGTCCCCCGATGACCCAGCCGGACTGCTCGATCTCAGACAGGGCATGT
TCGCTCAGCTTGTAGCCCAAAATGTCCTCCTGATTGACGGCCCTTTGAGCTGGTATAGTGATCC
CGGCTTGGCCGGGGTATCTCTGACCGGAGGCCTCTCCTACAAGGAAGACACCAAAGAGCTGGTG
GTGGCAAAAGCGGGGGTGTATTATGTGTTCTTTCAGCTCGAGCTGCGGAGAGTTGTGGCCGGGG
AAGGGTCTGGGAGCGTATCTCTTGCACTTCACCTGCAGCCCCTGCGCAGCGCCGCTGGAGCCGC
CGCCCTTGCTCTTACTGTGGATCTGCCTCCTGCTTCCTCAGAAGCACGCAACAGCGCCTTCGGC
TTTCAAGGACGTCTCCTGCACTTGTCCGCAGGACAGAGGTTGGGCGTCCATTTACACACTGAGG
CACGGGCACGGCACGCTTGGCAGCTTACCCAGGGAGCCACCGTGCTGGGACTCTTTAGAGTGAC
ACCCGAGATCCCCGCTGGCTTGTGA
>Sequence ID 115: NKG2D dimer amino acid sequence
FLNSLFNQEVQIPLTESYCGPCPKNWICYKNNCYQFFDESKNWYESQASCMSQNASLLKVYSKE
DQDLLKLVKSYHWMGLVHIPTNGSWQWEDGSILSPNLLTIIEMQKGDCALYASSFKGYIENCST
PNTYICMQRTVGGGGSGGGGSGGGGSGGGGSFLNSLFNQEVQIPLTESYCGPCPKNWICYKNNC
YQFFDESKNWYESQASCMSQNASLLKVYSKEDQDLLKLVKSYHWMGLVHIPTNGSWQWEDGSIL
SPNLLTIIEMQKGDCALYASSFKGYIENCSTPNTYICMQRTV
>Sequence ID 116: NKG2D dimer nucleotide sequence
TTTCTTAATTCCCTTTTCAACCAAGAGGTTCAGATCCCCTTGACTGAAAGCTATTGCGGCCCTT
GTCCGAAAAACTGGATATGTTACAAGAATAATTGTTACCAATTCTTCGACGAAAGCAAGAACTG
GTATGAGAGTCAGGCGTCTTGTATGAGTCAGAATGCCAGCCTGCTTAAGGTTTATTCAAAAGAA
GACCAGGATCTGCTTAAGTTGGTAAAGAGCTACCACTGGATGGGGCTGGTACATATCCCAACGA
ATGGGTCATGGCAGTGGGAGGACGGTTCTATTCTGAGTCCAAATCTCCTGACGATCATCGAAAT
GCAGAAAGGGGACTGTGCCCTGTATGCATCATCCTTCAAGGGGTACATCGAGAACTGCAGTACC
CCAAATACCTACATTTGTATGCAAAGAACGGTTGGAGGCGGTGGCTCAGGCGGAGGCGGCTCAG
GAGGTGGCGGTTCAGGAGGCGGCGGATCTTTCCTAAACTCATTATTCAACCAAGAAGTTCAAAT
TCCCTTGACCGAAAGTTACTGTGGCCCATGTCCTAAAAACTGGATATGTTACAAAAATAACTGC
TACCAATTTTTTGATGAGAGTAAAAACTGGTATGAGAGCCAGGCTTCTTGTATGTCTCAAAATG
CCAGCCTTCTGAAAGTATACAGCAAAGAGGACCAGGATTTACTTAAACTGGTGAAGTCATATCA
TTGGATGGGACTAGTACACATTCCAACAAATGGATCTTGGCAGTGGGAAGATGGCTCCATTCTC
TCACCCAACCTACTAACAATAATTGAAATGCAGAAGGGAGACTGTGCACTCTATGCCTCGAGCT
TTAAAGGCTATATAGAAAACTGTTCAACTCCAAATACGTACATCTGCATGCAAAGGACTGTGTA
G
>Sequence ID 117: SI-49P10 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVLGSTGSGSKPGSGEGST
KGEVQLVESGGGLVQPGGSLRLSCTASGFTISTNAMSWVRQAPGKCLEWVGVITGRDITYYASW
AKGRFTISRDTSKNTVYLQMNSLRAEDTAVYYCARDGGSSAITSNNIWGQGTLVTVSTGGGGSG
GGGSFLNSLFNQEVQIPLTESYCGPCPKNWICYKNNCYQFFDESKNWYESQASCMSQNASLLKV
YSKEDQDLLKLVKSYHWMGLVHIPTNGSWQWEDGSILSPNLLTIIEMQKGDCALYASSFKGYIE
NCSTPNTYICMQRTVASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCP
APEAAGGPSVFLFPPKPKDTLMISRTPEVTCVVVDVSHEDPEVKFNWYVDGVEVHNAKTKPREE
QYASTYRVVSVLTVLHQDWLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSEVQLLESGGGLVQPGGSLRLSCAASGFSFS
SGYDMCWVRQAPGKCLEWIACIAAGSAGITYDANWAKGRFTISRDNSKNTLYLQMNSLRAEDTA
VYYCARSAFSFDYAMDLWGQGTLVTVSSGGGGSGGGGSGGGGSGGGGSDIQMTQSPSTLSASVG
DRVTITCQASQSISSHLNWYQQKPGKAPKLLIYKASTLASGVPSRFSGSGSGTEFTLTISSLQP
DDFATYYCQQGYSWGNVDNVFGCGTKVEIKGGGGSGGGGSGRSLVESGGGLVQPGGSLRLSCTA
SGFTISSYHMQWVRQAPGKCLEYIGTISSGGNVYYASSARGRFTISRPSSKNTVDLQMNSLRAE
DTAVYYCARDSGYSDPMWGQGTLVTVSSGGGGSGGGGSGGGGSGGGGSDVVMTQSPSSVSASVG
DRVTITCQASQNIRTYLSWYQQKPGKAPKLLIYAAANLASGVPSRFSGSGSGTDFTLTISDLEP
GDAATYYCQSTYLGTDYVGGAFGCGTKVEIK
>Sequence ID 118: SI-49P10 heavy chain nucleotide sequence
GAGATCGTGATGACACAATCTCCATCTACGCTCTCCGCCTCAGTGGGCGATAGAGTAATTATTA
CTTGTCAAGCCTCAGAGAGCATTTCATCATGGCTCGCCTGGTATCAGCAAAAGCCTGGGAAGGC
CCCCAAACTTCTCATCTATGAAGCATCAAAGCTGGCCTCTGGGGTTCCGTCTCGCTTCTCCGGG
TCCGGCAGTGGTGCAGAGTTTACGTTGACTATATCTTCTTTGCAACCTGACGATTTCGCAACAT
ATTATTGCCAGGGATACTTTTATTTTATTTCCCGAACATATGTTAACTCTTTTGGGTGCGGGAC
CAAACTCACTGTGCTGGGGTCTACCGGTAGTGGTTCTAAGCCTGGTTCAGGCGAAGGCAGTACG
AAAGGGGAAGTGCAACTGGTCGAAAGCGGTGGAGGGCTTGTTCAACCTGGAGGAAGCCTCCGCT
TGTCCTGCACGGCTAGCGGCTTTACAATAAGTACGAACGCCATGAGCTGGGTCCGGCAGGCTCC
AGGTAAGTGTCTCGAATGGGTGGGGGTCATAACAGGCAGGGACATTACCTACTACGCCAGTTGG
GCCAAGGGTCGATTTACCATTTCTAGAGATACATCCAAGAACACGGTGTACCTCCAGATGAATT
CTCTTAGGGCGGAAGACACAGCAGTATACTACTGCGCGCGAGATGGCGGGAGCAGTGCGATCAC
ATCCAACAACATCTGGGGTCAGGGCACTCTTGTCACGGTGTCGACTGGTGGTGGGGGTAGTGGC
GGCGGAGGTAGCTTTCTTAATTCCCTTTTCAACCAAGAGGTTCAGATCCCCTTGACTGAAAGCT
ATTGCGGCCCTTGTCCGAAAAACTGGATATGTTACAAGAATAATTGTTACCAATTCTTCGACGA
AAGCAAGAACTGGTATGAGAGTCAGGCGTCTTGTATGAGTCAGAATGCCAGCCTGCTTAAGGTT
TATTCAAAAGAAGACCAGGATCTGCTTAAGTTGGTAAAGAGCTACCACTGGATGGGGCTGGTAC
ATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAG
AACTGCAGTACCCCAAATACCTACATTTGTATGCAAAGAACGGTTGCTAGCACCAAGGGCCCAT
CGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCA
GCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACGCCAGCACGTACCGTGTGGTCAGCGTCCTCACCGTCCTGCACCAGGACTGGCTGAATG
GCAAGGAGTACAAGTGCGCGGTCTCCAACAAAGCCCTCCCAGCCCCCATCGAGAAAACCATCTC
CAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGT
CTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGT
AGCGGGTACGACATGTGCTGGGTCCGCCAGGCTCCAGGGAAGTGCCTGGAGTGGATCGCATGCA
TTGCTGCTGGTAGTGCTGGTATCACTTACGACGCGAACTGGGCGAAAGGCCGGTTCACCATCTC
CAGAGACAATTCCAAGAACACGCTGTATCTGCAAATGAACAGCCTGAGAGCCGAGGACACGGCC
GTATATTACTGTGCGAGATCGGCGTTTTCGTTCGACTACGCCATGGACCTCTGGGGCCAGGGAA
CCCTGGTCACCGTCTCGAGCGGTGGAGGCGGTTCAGGCGGAGGTGGAAGTGGTGGTGGCGGCTC
TGGAGGCGGCGGATCTGACATCCAGATGACCCAGTCTCCTTCCACCCTGTCTGCATCTGTAGGA
GACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCC
ATCAAGGTTCAGCGGCAGTGGATCTGGGACAGAATTTACTCTCACCATCAGCAGCCTGCAGCCT
GATGATTTTGCAACTTATTACTGCCAACAGGGTTATAGTTGGGGTAATGTTGATAATGTTTTCG
GCTGCGGGACCAAGGTGGAGATCAAAGGTGGTGGCGGCTCTGGAGGAGGAGGGTCCGGACGGTC
GCTGGTGGAGTCTGGGGGAGGCTTGGTCCAGCCTGGGGGGTCCCTGAGACTCTCCTGTACTGCC
TCTGGATTCACCATCAGTAGCTACCACATGCAGTGGGTCCGGCAGGCACCTGGGAAGTGCCTGG
AGTACATCGGAACCATTAGTAGTGGTGGTAATGTATACTACGCAAGCTCCGCTAGAGGCAGATT
CACCATCTCCAGACCCTCGTCCAAGAACACGGTGGATCTTCAAATGAACAGCCTGAGAGCCGAG
GACACGGCTGTGTATTACTGTGCGAGAGACTCTGGTTATAGTGATCCTATGTGGGGCCAGGGAA
CCCTGGTCACCGTCTCTTCAGGCGGTGGCGGTAGTGGGGGAGGCGGTTCTGGCGGCGGAGGGTC
CGGCGGTGGAGGATCAGACGTTGTGATGACCCAGTCTCCATCTTCCGTGTCTGCATCTGTAGGA
GACAGAGTCACCATCACCTGTCAGGCCAGTCAGAACATTAGGACTTACTTATCCTGGTATCAGC
AGAAACCAGGGAAAGCCCCTAAGCTCCTGATCTATGCTGCAGCCAATCTGGCATCTGGGGTCCC
ATCAAGGTTCAGCGGCAGTGGATCTGGGACAGATTTCACTCTCACCATCAGCGACTTGGAACCT
GGCGATGCTGCAACTTACTATTGTCAGTCTACCTATCTTGGTACTGATTATGTTGGCGGTGCTT
TCGGCTGTGGGACCAAGGTGGAGATCAAATGA
>Sequence ID 119: SI-49P10 light chain moiety amino acid
sequence
DIQMTQSPSSLSASVGDRVTITCQASQDISNYLNWYQQKPGKAPKLLIYDASNLETGVPSRFSG
SGSGTDFTFTISSLQPEDIATYFCQHFDHLPLAFGGGTKVEIKGSTGSGSKPGSGEGSTKGQVQ
LQESGPGLVKPSETLSLTCTVSGGSVSSGDYYWTWIRQSPGKGLEWIGHIYYSGNTNYNPSLKS
RLTISIDTSKTQFSLKLSSVTAADTAIYYCVRDRVTGAFDIWGQGTMVTVSSGGGGSGGGGSGG
GGSFLNSLFNQEVQIPLTESYCGPCPKNWICYKNNCYQFFDESKNWYESQASCMSQNASLLKVY
SKEDQDLLKLVKSYHWMGLVHIPTNGSWQWEDGSILSPNLLTIIEMQKGDCALYASSFKGYIEN
CSTPNTYICMQRTVRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
>Sequence ID 120: SI-49P10 light chain moiety nucleotide
sequence
GACATCCAGATGACCCAGTCTCCATCCTCCCTGTCTGCATCTGTAGGAGACAGAGTCACCATCA
CTTGCCAGGCGAGTCAGGACATCAGCAACTATTTAAATTGGTATCAGCAGAAACCAGGGAAAGC
CCCTAAACTCCTGATCTACGATGCATCCAATTTGGAAACAGGGGTCCCATCAAGGTTCAGTGGA
AGTGGATCTGGGACAGATTTTACTTTCACCATCAGCAGCCTGCAGCCTGAAGATATTGCAACAT
ATTTCTGTCAACACTTTGATCATCTCCCGCTCGCTTTCGGCGGAGGGACCAAGGTGGAAATTAA
AGGAAGTACTGGTTCTGGGTCTAAACCCGGTTCCGGCGAAGGTAGTACTAAAGGACAGGTGCAG
CTGCAGGAGTCGGGCCCAGGACTGGTGAAGCCTTCGGAGACCCTGTCCCTCACCTGCACTGTCT
CTGGTGGCTCCGTCAGCAGTGGTGATTACTACTGGACCTGGATCCGGCAGTCCCCAGGGAAGGG
ACTGGAGTGGATTGGACACATCTATTACAGTGGGAACACCAATTATAACCCCTCCCTCAAGAGC
CGACTCACCATATCAATTGACACGTCCAAGACTCAGTTCTCCCTGAAGCTGAGTTCTGTGACCG
CTGCGGACACGGCCATTTATTACTGTGTGCGAGATCGAGTGACTGGTGCTTTTGATATCTGGGG
CCAAGGGACAATGGTCACCGTCTCGAGCGGTGGCGGCGGCTCCGGGGGTGGCGGATCAGGTGGT
GGAGGCTCTTTCCTAAACTCATTATTCAACCAAGAAGTTCAAATTCCCTTGACCGAAAGTTACT
GTGGCCCATGTCCTAAAAACTGGATATGTTACAAAAATAACTGCTACCAATTTTTTGATGAGAG
TAAAAACTGGTATGAGAGCCAGGCTTCTTGTATGTCTCAAAATGCCAGCCTTCTGAAAGTATAC
AGCAAAGAGGACCAGGATTTACTTAAACTGGTGAAGTCATATCATTGGATGGGACTAGTACACA
TTCCAACAAATGGATCTTGGCAGTGGGAAGATGGCTCCATTCTCTCACCCAACCTACTAACAAT
AATTGAAATGCAGAAGGGAGACTGTGCACTCTATGCCTCGAGCTTTAAAGGCTATATAGAAAAC
TGTTCAACTCCAAATACGTACATCTGCATGCAAAGGACTGTGCGTACGGTGGCTGCACCATCTG
TCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTAG
>Sequence ID 121: αCD19 SI-huBU12 VH amino acid sequence
QVTLKESGPGLVQPGQTLRLTCAFSGFSLSTSGMGVGWIRQPPGKGLEWLAHIWWDDDKRYNPA
LKSRLTISKDTSKNQVYLQMNSLDAEDTAVYYCARMELWSYYFDYWGQGTLVTVSS
>Sequence ID 122: αCD19 SI-huBU12 VH nucleotide sequence
CAGGTCACATTGAAGGAATCTGGCCCCGGCCTTGTTCAGCCAGGACAGACCCTTAGGCTCACCT
GTGCCTTCAGTGGTTTTTCTCTTAGCACTAGCGGTATGGGGGTCGGCTGGATTCGGCAGCCTCC
CGGCAAAGGTCTTGAGTGGTTGGCTCACATTTGGTGGGACGACGACAAACGGTATAATCCTGCC
TTGAAAAGTCGGCTGAGCATTAGTAAGGATACCTCAAAAAATCAAGTGTACTTGCAAATGAATA
GCCTTGACGCCGAGGATACGGCTGTATATTATTGCGCGCGGATGGAACTCTGGTCTTACTACTT
TGATTATTGGGGGCAGGGGACTCTCGTCACGGTCTCGAGC
>Sequence ID 123: SI-49P6 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVLGSTGSGSKPGSGEGST
KGEVQLVESGGGLVQPGGSLRLSCTASGFTISTNAMSWVRQAPGKCLEWVGVITGRDITYYASW
AKGRFTISRDTSKNTVYLQMNSLRAEDTAVYYCARDGGSSAITSNNIWGQGTLVTVSTGGGGSG
GGGSFLNSLFNQEVQIPLTESYCGPCPKNWICYKNNCYQFFDESKNWYESQASCMSQNASLLKV
YSKEDQDLLKLVKSYHWMGLVHIPTNGSWQWEDGSILSPNLLTIIEMQKGDCALYASSFKGYIE
NCSTPNTYICMQRTVASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCP
APEAAGGPSVFLFPPKPKDTLMISRTPEVTCVVVDVSHEDPEVKFNWYVDGVEVHNAKTKPREE
QYASTYRVVSVLTVLHQDWLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSEVQLLESGGGLVQPGGSLRLSCAASGFSFS
SGYDMCWVRQAPGKCLEWIACIAAGSAGITYDANWAKGRFTISRDNSKNTLYLQMNSLRAEDTA
VYYCARSAFSFDYAMDLWGQGTLVTVSSGGGGSGGGGSGGGGSGGGGSDIQMTQSPSTLSASVG
DRVTITCQASQSISSHLNWYQQKPGKAPKLLIYKASTLASGVPSRFSGSGSGTEFTLTISSLQP
DDFATYYCQQGYSWGNVDNVFGCGTKVEIKGGGGSGGGGSGRSLVESGGGLVQPGGSLRLSCTA
SGFTISSYHMQWVRQAPGKCLEYIGTISSGGNVYYASSARGRFTISRPSSKNTVDLQMNSLRAE
DTAVYYCARDSGYSDPMWGQGTLVTVSSGGGGSGGGGSGGGGSGGGGSDVVMTQSPSSVSASVG
DRVTITCQASQNIRTYLSWYQQKPGKAPKLLIYAAANLASGVPSRFSGSGSGTDFTLTISDLEP
GDAATYYCQSTYLGTDYVGGAFGCGTKVEIK
>Sequence ID 124: SI-49P6 heavy chain nucleotide sequence
GAGATCGTGATGACACAATCTCCATCTACGCTCTCCGCCTCAGTGGGCGATAGAGTAATTATTA
CTTGTCAAGCCTCAGAGAGCATTTCATCATGGCTCGCCTGGTATCAGCAAAAGCCTGGGAAGGC
CCCCAAACTTCTCATCTATGAAGCATCAAAGCTGGCCTCTGGGGTTCCGTCTCGCTTCTCCGGG
TCCGGCAGTGGTGCAGAGTTTACGTTGACTATATCTTCTTTGCAACCTGACGATTTCGCAACAT
ATTATTGCCAGGGATACTTTTATTTTATTTCCCGAACATATGTTAACTCTTTTGGGTGCGGGAC
CAAACTCACTGTGCTGGGGTCTACCGGTAGTGGTTCTAAGCCTGGTTCAGGCGAAGGCAGTACG
AAAGGGGAAGTGCAACTGGTCGAAAGCGGTGGAGGGCTTGTTCAACCTGGAGGAAGCCTCCGCT
TGTCCTGCACGGCTAGCGGCTTTACAATAAGTACGAACGCCATGAGCTGGGTCCGGCAGGCTCC
AGGTAAGTGTCTCGAATGGGTGGGGGTCATAACAGGCAGGGACATTACCTACTACGCCAGTTGG
GCCAAGGGTCGATTTACCATTTCTAGAGATACATCCAAGAACACGGTGTACCTCCAGATGAATT
CTCTTAGGGCGGAAGACACAGCAGTATACTACTGCGCGCGAGATGGCGGGAGCAGTGCGATCAC
ATCCAACAACATCTGGGGTCAGGGCACTCTTGTCACGGTGTCGACTGGTGGTGGGGGTAGTGGC
GGCGGAGGTAGCTTTCTTAATTCCCTTTTCAACCAAGAGGTTCAGATCCCCTTGACTGAAAGCT
ATTGCGGCCCTTGTCCGAAAAACTGGATATGTTACAAGAATAATTGTTACCAATTCTTCGACGA
AAGCAAGAACTGGTATGAGAGTCAGGCGTCTTGTATGAGTCAGAATGCCAGCCTGCTTAAGGTT
TATTCAAAAGAAGACCAGGATCTGCTTAAGTTGGTAAAGAGCTACCACTGGATGGGGCTGGTAC
ATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAG
AACTGCAGTACCCCAAATACCTACATTTGTATGCAAAGAACGGTTGCTAGCACCAAGGGCCCAT
CGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCA
GCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACGCCAGCACGTACCGTGTGGTCAGCGTCCTCACCGTCCTGCACCAGGACTGGCTGAATG
GCAAGGAGTACAAGTGCGCGGTCTCCAACAAAGCCCTCCCAGCCCCCATCGAGAAAACCATCTC
CAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGT
CTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGT
AGCGGGTACGACATGTGCTGGGTCCGCCAGGCTCCAGGGAAGTGCCTGGAGTGGATCGCATGCA
TTGCTGCTGGTAGTGCTGGTATCACTTACGACGCGAACTGGGCGAAAGGCCGGTTCACCATCTC
CAGAGACAATTCCAAGAACACGCTGTATCTGCAAATGAACAGCCTGAGAGCCGAGGACACGGCC
GTATATTACTGTGCGAGATCGGCGTTTTCGTTCGACTACGCCATGGACCTCTGGGGCCAGGGAA
CCCTGGTCACCGTCTCGAGCGGTGGAGGCGGTTCAGGCGGAGGTGGAAGTGGTGGTGGCGGCTC
TGGAGGCGGCGGATCTGACATCCAGATGACCCAGTCTCCTTCCACCCTGTCTGCATCTGTAGGA
GACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCC
ATCAAGGTTCAGCGGCAGTGGATCTGGGACAGAATTTACTCTCACCATCAGCAGCCTGCAGCCT
GATGATTTTGCAACTTATTACTGCCAACAGGGTTATAGTTGGGGTAATGTTGATAATGTTTTCG
GCTGCGGGACCAAGGTGGAGATCAAAGGTGGTGGCGGCTCTGGAGGAGGAGGGTCCGGACGGTC
GCTGGTGGAGTCTGGGGGAGGCTTGGTCCAGCCTGGGGGGTCCCTGAGACTCTCCTGTACTGCC
TCTGGATTCACCATCAGTAGCTACCACATGCAGTGGGTCCGGCAGGCACCTGGGAAGTGCCTGG
AGTACATCGGAACCATTAGTAGTGGTGGTAATGTATACTACGCAAGCTCCGCTAGAGGCAGATT
CACCATCTCCAGACCCTCGTCCAAGAACACGGTGGATCTTCAAATGAACAGCCTGAGAGCCGAG
GACACGGCTGTGTATTACTGTGCGAGAGACTCTGGTTATAGTGATCCTATGTGGGGCCAGGGAA
CCCTGGTCACCGTCTCTTCAGGCGGTGGCGGTAGTGGGGGAGGCGGTTCTGGCGGCGGAGGGTC
CGGCGGTGGAGGATCAGACGTTGTGATGACCCAGTCTCCATCTTCCGTGTCTGCATCTGTAGGA
GACAGAGTCACCATCACCTGTCAGGCCAGTCAGAACATTAGGACTTACTTATCCTGGTATCAGC
AGAAACCAGGGAAAGCCCCTAAGCTCCTGATCTATGCTGCAGCCAATCTGGCATCTGGGGTCCC
ATCAAGGTTCAGCGGCAGTGGATCTGGGACAGATTTCACTCTCACCATCAGCGACTTGGAACCT
GGCGATGCTGCAACTTACTATTGTCAGTCTACCTATCTTGGTACTGATTATGTTGGCGGTGCTT
TCGGCTGTGGGACCAAGGTGGAGATCAAATGA
>Sequence ID 125: SI-49P6 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKLEIKGSTGSGSKPGSGEGSTKGQVTL
KESGPGLVQPGQTLRLTCAFSGFSLSTSGMGVGWIRQPPGKGLEWLAHIWWDDDKRYNPALKSR
LTISKDTSKNQVYLQMNSLDAEDTAVYYCARMELWSYYFDYWGQGTLVTVSSGGGGSGGGGSGG
GGSFLNSLFNQEVQIPLTESYCGPCPKNWICYKNNCYQFFDESKNWYESQASCMSQNASLLKVY
SKEDQDLLKLVKSYHWMGLVHIPTNGSWQWEDGSILSPNLLTIIEMQKGDCALYASSFKGYIEN
CSTPNTYICMQRTVRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
>Sequence ID 126: SI-49P6 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAATTGGAGATAAAGGG
AAGTACTGGTTCTGGGTCTAAACCCGGTTCCGGCGAAGGTAGTACTAAAGGACAGGTCACATTG
AAGGAATCTGGCCCCGGCCTTGTTCAGCCAGGACAGACCCTTAGGCTCACCTGTGCCTTCAGTG
GTTTTTCTCTTAGCACTAGCGGTATGGGGGTCGGCTGGATTCGGCAGCCTCCCGGCAAAGGTCT
TGAGTGGTTGGCTCACATTTGGTGGGACGACGACAAACGGTATAATCCTGCCTTGAAAAGTCGG
CTGACCATTAGTAAGGATACCTCAAAAAATCAAGTGTACTTGCAAATGAATAGCCTTGACGCCG
AGGATACGGCTGTATATTATTGCGCGCGGATGGAACTCTGGTCTTACTACTTTGATTATTGGGG
GCAGGGGACTCTCGTCACGGTGTCCTCTGGTGGCGGCGGCTCCGGGGGTGGCGGATCAGGTGGT
GGAGGATCCTTCCTAAACTCATTATTCAACCAAGAAGTTCAAATTCCCTTGACCGAAAGTTAGT
GTGGCCCATGTCCTAAAAACTGGATATGTTACAAAAATAACTGCTACCAATTTTTTGATGAGAG
TAAAAACTGGTATGAGAGCCAGGCTTCTTGTATGTCTCAAAATGCCAGCCTTCTGAAAGTATAC
AGCAAAGAGGACCAGGATTTACTTAAACTGGTGAAGTCATATCATTGGATGGGACTAGTACACA
TTCCAACAAATGGATCTTGGCAGTGGGAAGATGGCTCCATTCTCTCACCCAACCTACTAACAAT
AATTGAAATGCAGAAGGGAGACTGTGCACTCTATGCCTCGAGCTTTAAAGGCTATATAGAAAAC
TGTTCAACTCCAAATACGTACATCTGCATGCAAAGGACTGTGCGTACGGTGGCTGCACCATCTG
TCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTAG
>Sequence ID 127: SI-49P7 heavy chain amino acid sequence
EIVMTQSPSTLSASVGDRVIITCQASESISSWLAWYQQKPGKAPKLLIYEASKLASGVPSRFSG
SGSGAEFTLTISSLQPDDFATYYCQGYFYFISRTYVNSFGCGTKLTVLGSTGSGSKPGSGEGST
KGEVQLVESGGGLVQPGGSLRLSCTASGFTISTNAMSWVRQAPGKCLEWVGVITGRDITYYASW
AKGRFTISRDTSKNTVYLQMNSLRAEDTAVYYCARDGGSSAITSNNIWGQGTLVTVSTGGGGSG
GGGSFLNSLFNQEVQIPLTESYCGPCPKNWICYKNNCYQFFDESKNWYESQASCMSQNASLLKV
YSKEDQDLLKLVKSYHWMGLVHIPTNGSWQWEDGSILSPNLLTIIEMQKGDCALYASSFKGYIE
NCSTPNTYICMQRTVASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCP
APEAAGGPSVFLFPPKPKDTLMISRTPEVTCVVVDVSHEDPEVKFNWYVDGVEVHNAKTKPREE
QYASTYRVVSVLTVLHQDWLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSEVQLLESGGGLVQPGGSLRLSCAASGFSFS
SGYDMCWVRQAPGKCLEWIACIAAGSAGITYDANWAKGRFTISRDNSKNTLYLQMNSLRAEDTA
VYYCARSAFSFDYAMDLWGQGTLVTVSSGGGGSGGGGSGGGGSGGGGSDIQMTQSPSTLSASVG
DRVTITCQASQSISSHLNWYQQKPGKAPKLLIYKASTLASGVPSRFSGSGSGTEFTLTISSLQP
DDFATYYCQQGYSWGNVDNVFGCGTKVEIKGGGGSGGGGSGREGPELSPDDPAGLLDLRQGMFA
QLVAQNVLLIDGPLSWYSDPGLAGVSLTGGLSYKEDTKELVVAKAGVYYVFFQLELRRVVAGEG
SGSVSLALHLQPLRSAAGAAALALTVDLPPASSEARNSAFGFQGRLLHLSAGQRLGVHLHTEAR
ARHAWQLTQGATVLGLFRVTPEIPAGLGSTGSGSKPGSGEGSTKGREGPELSPDDPAGLLDLRQ
GMFAQLVAQNVLLIDGPLSWYSDPGLAGVSLTGGLSYKEDTKELVVAKAGVYYVFFQLELRRVV
AGEGSGSVSLALHLQPLRSAAGAAALALTVDLPPASSEARNSAFGFQGRLLHLSAGQRLGVHLH
TEARARHAWQLTQGATVLGLFRVTPEIPAGLGGGGSGGGGSREGPELSPDDPAGLLDLRQGMFA
QLVAQNVLLIDGPLSWYSDPGLAGVSLTGGLSYKEDTKELVVAKAGVYYVFFQLELRRVVAGEG
SGSVSLALHLQPLRSAAGAAALALTVDLPPASSEARNSAFGFQGRLLHLSAGQRLGVHLHTEAR
ARHAWQLTQGATVLGLFRVTPEIPAGL
>Sequence ID 128: SI-49P7 heavy chain nucleotide sequence
GAGATCGTGATGACACAATCTCCATCTACGCTCTCCGCCTCAGTGGGCGATAGAGTAATTATTA
CTTGTCAAGCCTCAGAGAGCATTTCATCATGGCTCGCCTGGTATCAGCAAAAGCCTGGGAAGGC
CCCCAAACTTCTCATCTATGAAGCATCAAAGCTGGCCTCTGGGGTTCCGTCTCGCTTCTCCGGG
TCCGGCAGTGGTGCAGAGTTTACGTTGACTATATCTTCTTTGCAACCTGACGATTTCGCAACAT
ATTATTGCCAGGGATACTTTTATTTTATTTCCCGAACATATGTTAACTCTTTTGGGTGCGGGAC
CAAACTCACTGTGCTGGGGTCTACCGGTAGTGGTTCTAAGCCTGGTTCAGGCGAAGGCAGTACG
AAAGGGGAAGTGCAACTGGTCGAAAGCGGTGGAGGGCTTGTTCAACCTGGAGGAAGCCTCCGCT
TGTCCTGCACGGCTAGCGGCTTTACAATAAGTACGAACGCCATGAGCTGGGTCCGGCAGGCTCC
AGGTAAGTGTCTCGAATGGGTGGGGGTCATAACAGGCAGGGACATTACCTACTACGCCAGTTGG
GCCAAGGGTCGATTTACCATTTCTAGAGATACATCCAAGAACACGGTGTACCTCCAGATGAATT
CTCTTAGGGCGGAAGACACAGCAGTATACTACTGCGCGCGAGATGGCGGGAGCAGTGCGATCAC
ATCCAACAACATCTGGGGTCAGGGCACTCTTGTCACGGTGTCGACTGGTGGTGGGGGTAGTGGC
GGCGGAGGTAGCTTTCTTAATTCCCTTTTCAACCAAGAGGTTCAGATCCCCTTGACTGAAAGCT
ATTGCGGCCCTTGTCCGAAAAACTGGATATGTTACAAGAATAATTGTTACCAATTCTTCGACGA
AAGCAAGAACTGGTATGAGAGTCAGGCGTCTTGTATGAGTCAGAATGCCAGCCTGCTTAAGGTT
TATTCAAAAGAAGACCAGGATCTGCTTAAGTTGGTAAAGAGCTACCACTGGATGGGGCTGGTAC
ATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAG
AACTGCAGTACCCCAAATACCTACATTTGTATGCAAAGAACGGTTGCTAGCACCAAGGGCCCAT
CGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAATCACAAGCCCAGCAACAC
CAAGGTGGACAAGAGAGTTGAGCCCAAATCTTGTGACAAAACTCACACATGCCCACCGTGCCCA
GCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGT
CAAGTTCAACTGGTACGTGGACGGCGTGGAGGTGCATAATGCCAAGACAAAGCCGCGGGAGGAG
CAGTACGCCAGCACGTACCGTGTGGTCAGCGTCCTCACCGTCCTGCACCAGGACTGGCTGAATG
GCAAGGAGTACAAGTGCGCGGTCTCCAACAAAGCCCTCCCAGCCCCCATCGAGAAAACCATCTC
CAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCGA
CGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGT
CTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGT
AGCGGGTACGACATGTGCTGGGTCCGCCAGGCTCCAGGGAAGTGCCTGGAGTGGATCGCATGCA
TTGCTGCTGGTAGTGCTGGTATCACTTACGACGCGAACTGGGCGAAAGGCCGGTTCACCATCTC
CAGAGACAATTCCAAGAACACGCTGTATCTGCAAATGAACAGCCTGAGAGCCGAGGACACGGCC
GTATATTACTGTGCGAGATCGGCGTTTTCGTTCGACTACGCCATGGACCTCTGGGGCCAGGGAA
CCCTGGTCACCGTCTCGAGCGGTGGAGGCGGTTCAGGCGGAGGTGGAAGTGGTGGTGGCGGCTC
TGGAGGCGGCGGATCTGACATCCAGATGACCCAGTCTCCTTCCACCCTGTCTGCATCTGTAGGA
GACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCC
ATCAAGGTTCAGCGGCAGTGGATCTGGGACAGAATTTACTCTCACCATCAGCAGCCTGCAGCCT
GATGATTTTGCAACTTATTACTGCCAACAGGGTTATAGTTGGGGTAATGTTGATAATGTTTTCG
GCTGCGGGACCAAGGTGGAGATCAAAGGTGGTGGCGGCTCTGGAGGAGGAGGGTCCGGACGAGA
GGGCCCCGAGCTGTCTCCTGATGACCCAGCAGGCCTCTTGGACTTGCGGCAGGGTATGTTCGCT
CAACTTGTGGCTCAGAATGTTCTGCTCATTGATGGACCACTCTCTTGGTATAGTGACCCCGGTC
TGGCCGGGGTGAGTCTGACCGGCGGGCTCTCTTATAAAGAGGATACTAAGGAACTGGTCGTAGC
AAAAGCGGGCGTTTATTACGTTTTTTTTCAGCTGGAGCTCAGGCGCGTGGTGGCCGGCGAGGGC
AGTGGCTCTGTGTCCCTGGCCCTGCACTTACAGCCCTTGAGAAGCGCTGCAGGTGCTGCCGCAC
TGGCTTTAACTGTTGACCTCCCTCCGGCCTCTTCTGAAGCTAGAAACAGCGCTTTCGGCTTCCA
AGGGCGCCTGCTGCACCTGAGCGCAGGCCAGCGCTTAGGTGTGCACCTTCATACAGAGGCCAGG
GCCCGACACGCTTGGCAGCTCACACAGGGTGCCACGGTTCTCGGACTTTTCCGCGTTACTCCCG
AGATCCCCGCTGGCCTCGGAAGTACTGGTTCTGGGTCTAAACCCGGTTCCGGCGAAGGTAGTAC
TAAAGGACGAGAAGGGCCAGAGTTAAGTCCAGATGACCCTGCTGGGCTTTTGGACCTGCGGCAG
GGCATGTTCGCTCAACTGGTGGCTCAGAACGTGCTGCTGATCGATGGCCCCCTGAGTTGGTACA
GCGATCCCGGGCTGGCAGGCGTGTCACTTACAGGGGGCCTCTCTTACAAGGAAGACACCAAGGA
GTTAGTGGTCGCTAAGGCTGGCGTGTATTACGTGTTCTTCCAACTGGAGCTGAGAAGGGTTGTG
GCAGGAGAGGGTAGCGGCAGCGTGTCTTTAGCCCTTCACTTGCAGCCCCTGAGGTCTGCTGCAG
GTGCAGCCGCTCTCGCGCTCACCGTGGATCTCCCCCCAGCCTCATCTGAAGCTAGGAACAGTGC
ATTTGGCTTTCAGGGACGCTTGCTGCACCTCTCCGCTGGACAGAGGCTGGGCGTGCACCTTCAC
ACAGAGGCCCGTGCCAGGCATGCATGGCAGCTCACTCAGGGGGCAACAGTGCTGGGTCTCTTCC
GCGTGACTCCTGAAATACCAGCTGGACTTGGCGGTGGAGGCAGCGGCGGAGGAGGATCTCGTGA
GGGGCCAGAACTGTCCCCCGATGACCCAGCCGGACTGCTCGATCTCAGACAGGGCATGTTCGCT
CAGCTTGTAGCCCAAAATGTCCTCCTGATTGACGGCCCTTTGAGCTGGTATAGTGATCCCGGCT
TGGCCGGGGTATCTCTGACCGGAGGCCTCTCCTACAAGGAAGACACCAAAGAGCTGGTGGTGGC
AAAAGCGGGGGTGTATTATGTGTTCTTTCAGCTCGAGCTGCGGAGAGTTGTGGCCGGGGAAGGG
TCTGGGAGCGTATCTCTTGCACTTCACCTGCAGCCCCTGCGCAGCGCCGCTGGAGCCGCCGCCC
TTGCTCTTACTGTGGATCTGCCTCCTGCTTCCTCAGAAGCACGCAACAGCGCCTTCGGCTTTCA
AGGACGTCTCCTGCACTTGTCCGCAGGACAGAGGTTGGGCGTCCATTTACACACTGAGGCACGG
GCACGGCACGCTTGGCAGCTTACCCAGGGAGCCACCGTGCTGGGACTCTTTAGAGTGACACCCG
AGATCCCCGCTGGCTTGTGA
>Sequence ID 129: SI-49P7 light chain moiety amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKLEIKGSTGSGSKPGSGEGSTKGQVTL
KESGPGLVQPGQTLRLTCAFSGFSLSTSGMGVGWIRQPPGKGLEWLAHIWWDDDKRYNPALKSR
LTISKDTSKNQVYLQMNSLDAEDTAVYYCARMELWSYYFDYWGQGTLVTVSSGGGGSGGGGSGG
GGSFLNSLFNQEVQIPLTESYCGPCPKNWICYKNNCYQFFDESKNWYESQASCMSQNASLLKVY
SKEDQDLLKLVKSYHWMGLVHIPTNGSWQWEDGSILSPNLLTIIEMQKGDCALYASSFKGYIEN
CSTPNTYICMQRTVRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
>Sequence ID 130: SI-49P7 light chain moiety nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAATTGGAGATAAAGGG
AAGTACTGGTTCTGGGTCTAAACCCGGTTCCGGCGAAGGTAGTACTAAAGGACAGGTCACATTG
AAGGAATCTGGCCCCGGCCTTGTTCAGCCAGGACAGACCCTTAGGCTCACCTGTGCCTTCAGTG
GTTTTTCTCTTAGCACTAGCGGTATGGGGGTCGGCTGGATTCGGCAGCCTCCCGGCAAAGGTCT
TGAGTGGTTGGCTCACATTTGGTGGGACGACGACAAACGGTATAATCCTGCCTTGAAAAGTCGG
CTGACCATTAGTAAGGATACCTCAAAAAATCAAGTGTACTTGCAAATGAATAGCCTTGACGCCG
AGGATACGGCTGTATATTATTGCGCGCGGATGGAACTCTGGTCTTACTACTTTGATTATTGGGG
GCAGGGGACTCTCGTCACGGTGTCCTCTGGTGGCGGCGGCTCCGGGGGTGGCGGATCAGGTGGT
GGAGGATCCTTCCTAAACTCATTATTCAACCAAGAAGTTCAAATTCCCTTGACCGAAAGTTACT
GTGGCCCATGTCCTAAAAACTGGATATGTTACAAAAATAACTGCTACCAATTTTTTGATGAGAG
TAAAAACTGGTATGAGAGCCAGGCTTCTTGTATGTCTCAAAATGCCAGCCTTCTGAAAGTATAC
AGCAAAGAGGACCAGGATTTACTTAAACTGGTGAAGTCATATCATTGGATGGGACTAGTACACA
TTCCAACAAATGGATCTTGGCAGTGGGAAGATGGCTCCATTCTCTCACCCAACCTACTAACAAT
AATTGAAATGCAGAAGGGAGACTGTGCACTCTATGCCTCGAGCTTTAAAGGCTATATAGAAAAC
TGTTCAACTCCAAATACGTACATCTGCATGCAAAGGACTGTGCGTACGGTGGCTGCACCATCTG
TCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGT
AACTCCCAGGAGAGTGTCACAGAGCAGGACAGCAAGGACAGCACCTACAGCCTCAGCAGCACCC
TGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTAG
>Sequence ID 131: αCD19 SI-huBU12 VL amino acid sequence
ENVLTQSPASLSASPGERVTITCSASSSVSYMHWYQQKPGQAPKLWIYDTSKLASGVPSRFSGS
GSGNDHTLTISSMEPEDFATYYCFQGSVYPFTFGQGTKLEIK
>Sequence ID 132: αCD19 SI-huBU12 VL nucleotide sequence
GAAAATGTATTGACACAGAGCCCCGCCTCCCTCAGTGCCTCACCTGGGGAAAGGGTAACTATCA
CTTGCTCTGCATCAAGCAGCGTCTCATACATGCATTGGTATCAACAAAAGCCTGGACAGGCCCC
CAAGCTCTGGATATACGATACGAGCAAGCTGGCTTCCGGCGTACCTAGCCGCTTCAGTGGTTCC
GGCTCAGGCAACGATCACACCCTTACGATTTCCAGTATGGAACCCGAAGATTTTGCAACTTATT
ATTGTTTCCAGGGGAGCGTGTACCCATTCACTTTCGGGCAGGGGACAAAATTGGAGATAAAG

What is claimed is:

1. A multi-specific antibody-like protein having a N-terminal and a C-terminal, comprising in tandem from the N-terminal to the C-terminal,
a first binding domain (D1) at the N-terminal,
a second binding domain (D2) comprising a light chain moiety,
a Fc region,
a third binding domain (D3), and
a fourth binding domain (D4) at the C-terminal,
wherein the light chain moiety comprises a fifth binding domain (D5) covalently attached to the C-terminal, a sixth binding domain (D6) covalently attached to the N-terminal, or both, and
wherein the D1, D2, D3, D4, D5 and D6 each has a binding specificity to a tumor antigen, an immune signaling antigen, or a combination thereof.

2. The multi-specific antibody-like protein of claim 1, wherein the D2 comprises a dimer connected to CL and CH1, a Fab region, or a receptor.

3-4. (canceled)

5. The multi-specific antibody-like protein of claim 1, wherein the D2 comprises NKG2D, or wherein the D2 has a binding specificity to CD3 or a tumor associated antigen (TAA).

6. The multi-specific antibody-like protein of claim 1, wherein the light chain moiety comprises a fifth binding domain (D5) covalently attached to the C-terminal, and wherein the multi-specific antibody-like protein is penta-specific.

7. The multi-specific antibody-like protein of claim 1, wherein the light chain moiety comprises a sixth binding domain (D6) covalently attached to the N-terminal, and wherein the multi-specific antibody-like protein is penta-specific.

8. The multi-specific antibody-like protein of claim 1, wherein the light chain moiety comprises a fifth binding domain (D5) covalently attached to the C-terminal and a sixth binding domain (D6) covalently attached to the N-terminal, and wherein the multi-specific antibody-like protein is hexa-specific.

9-10. (canceled)

11. The multi-specific antibody-like protein of claim 1, wherein the D1, D2, D3, D4, D5, and D6 is independently a scFv domain, a receptor, or a ligand, or wherein the D1, D2, D3, D4, D5, and D6 independently has a binding specificity to an antigen selected from a receptor on a T cell, an immune checkpoint receptor, a co-stimulation receptor, a receptor of a lymphocyte or a myeloid cell, a tumor associated antigen (TAA), a tissue antigen, a neoantigen, a tumor-specific antigen (TSA), a glycoprotein, or a combination thereof.

12. The multi-specific antibody-like protein of claim 11, wherein the binding domain for the receptor on the T cell is adjacent to the binding domain for the tumor associated antigen (TAA).

13. The multi-specific antibody-like protein of claim 11, wherein the binding domain for the receptor on the T cell is adjacent to the binding domain for the receptor of a lymphocyte or a myeloid cell.

14. The multi-specific antibody-like protein of claim 11, wherein the receptor on the T cell comprises CD3, T cell receptor, or a complex thereof, wherein the immune checkpoint receptor comprises PD-L1, PD-1, TIGIT, TIM-3, LAG-3, CTLA4, BTLA, VISTA, PDL2, CD160, LOX-1, siglec-15, CD47, SIRPα, or a combination thereof, wherein the co-stimulating receptor comprises 4-1BB, CD28, OX40, GITR, CD40, ICOS, CD27, CD30, CD226, or a combination thereof, or wherein the tumor associated antigen (TAA) comprises EGFR, HER2, HER3, HER4, EGRFVIII, CD19, claudin 18.2, BCMA, CD20, CD33, CD123, CD22, CD30, ROR1, CEA, cMET, LMP1, LMP2A, Mesothelin, PSMA, EpCAM, glypican-3, gpA33, GD2, TACI, TROP2, NKG2D ligands, PD-L1, or a combination thereof.

15-17. (canceled)

18. The multi-specific antibody-like protein of claim 1, wherein the D1 has a binding specificity to CD3, CD20, EGFR, or their derivative thereof.

19. The multi-specific antibody-like protein of claim 1, wherein the D2 has the binding specificity to EGFR, CD3, HER2, MSLN, NKG2D ligands, or their derivative thereof.

20. The multi-specific antibody-like protein of claim 1, wherein the D3 has a binding specificity to PD-L1.

21. The multi-specific antibody-like protein of claim 1, wherein the D4 comprise a 4-1BBL trimer or has a binding specificity to 4-1BB or its derivative thereof.

22. The multi-specific antibody-like protein of claim 1, wherein the D5 has a binding specificity to HER3, CD19, NKG2D ligands, or their derivative thereof.

23. The multi-specific antibody-like protein of claim 1, wherein the D6 has a binding specificity to CD19.

24. (canceled)

25. A guidance and navigation control protein, comprising a dimer of the multi-specific antibody-like protein of claim 1.

26. An isolated nucleic acid sequence, encoding an amino acid sequence of the multi-specific antibody-like protein of claim 1.

27. An expression vector, comprising the isolated nucleic acid sequence of claim 26.

28. A host cell comprising the isolated nucleic acid sequence of claim 26, wherein the host cell is a prokaryotic cell or a eukaryotic cell.

29. A method for producing a multi-specific antibody or monomer, comprising culturing a host cell comprising an isolated nucleic acid sequence such that the DNA sequence encoding the multi-specific antibody-like protein of claim 1 is expressed, and purifying said multi-specific antibody-like protein.

30. A method for treating or preventing a cancer, an autoimmune disease, or an infectious disease, said method comprising administering a pharmaceutical composition comprising a purified multi-specific antibody of claim 29.

31. An immuno-conjugate comprising a cytotoxic agent or an imaging agent linked to the multi-specific antibody of claim 29 through a linker, wherein the linker comprises an ester bond, an ether bond, an amid bond, a disulphide bond, an imide bond, a sulfone bond, a phosphate bond, a phosphorus ester bond, a peptide bond, a hydrophobic poly(ethylene glycol) linker, or a combination thereof.

32. A pharmaceutical composition, comprising a pharmaceutically acceptable carrier and one of the multi-specific antibodies of claim 29, the immuno-conjugate of claim 31, or both.
